(19) United States
(12) Patent Application Publication
(10) Pub. No.: US 2021/0238618 A1
(43) Pub. Date: Aug. 5, 2021

(54) COMPOSITIONS AND METHODS FOR REDUCING CAFFEINE CONTENT IN COFFEE BEANS

(71) Applicant: Tropic Biosciences UK Limited, Norwich (GB)

(72) Inventors: Eyal MAORI, Rishon-LeZion (IL); Cristina PIGNOCCHI, Norwich (GB); Agnieska SIWOSZEK, Norwich (GB); Yaron GALANTY, Cambridge (GB); Daniel KNEVITT, Norwich (GB); Angela CHAPARRO GARCIA, Norwich (GB); Ofir MEIR, Norwich (GB)

(73) Assignee: Tropic Biosciences UK Limited, Norwich (GB)

(21) Appl. No.: 17/051,724

(22) PCT Filed: Apr. 30, 2019
(86) PCT No.: PCT/IB2019/053538

§ 371 Date: Oct. 29, 2020

(30) Foreign Application Priority Data
May 1, 2018 (GB) 1807192.8

(51) Int. Cl. C12N 15/82; C12N 9/22; A01H 1/00

(52) U.S. Cl. CPC C12N 15/8243; A01H 1/101; C12N 9/22; C12N 15/8213

(57) ABSTRACT

A coffee plant comprising a genome comprising a loss of function mutation in a nucleic acid sequence encoding at least one component of a caffeine biosynthesis pathway is disclosed. Methods of producing a coffee plant or part thereof, methods of producing coffee beans with reduced caffeine content, and methods of producing coffee with reduced caffeine content are also disclosed.

### RELATED APPLICATION/S

This application claims the benefit of priority of United Kingdom Provisional patent Application No. 1807192.8 filed on May 1, 2018, the contents of which are incorporated herein by reference in their entirety.

### SEQUENCE LISTING STATEMENT

The ASCII file, entitled 73882 Sequence Listing.txt, created on 30 Apr. 2019, comprising 92,812 bytes, submitted concurrently with the filing of this application is incorporated herein by reference.

### FIELD AND BACKGROUND OF THE INVENTION

The present invention, in some embodiments thereof, relates to compositions and methods for reducing caffeine content in coffee beans.

Coffea canephora (Robusta coffee) is one of two Coffea species that are commercially grown for their seeds which are harvested and processed to create the popular beverage coffee. Coffee is consumed worldwide and contains the stimulant caffeine which naturally accumulates in the coffee plant and appears within the beverage in moderate levels. Caffeine, although desirable for the majority of consumers is something which a significant few wish to avoid. The sale of coffee with a reduced caffeine content currently accounts for $1.6bn (about 7% of the market). Various methods are currently employed to commercially produce decaffeinated coffee, all of which are post-harvest process. Although much research and development has been performed to optimise these processes, they are unable to remove caffeine from the unroasted bean without affecting other components which contribute flavour to the final beverage.

Caffeine is a purine alkaloid, it is a secondary metabolite derived from purine metabolism. Xanthosine from purine metabolism undergoes three methylation steps and removal of a ribose residue to form caffeine. These methylation steps are attributed to three methyltransferases, xanthosine methyltransferase (XMT), 7-methyxanthine methyltrasferase (MXMT or theobromine synthase), and 3,7-dimethylxanthine methyltransferase (DXMT or caffeine synthase).

The first step is methylation of xanthosine by XMT which yields 7-methyxanthosine (FIG. 1, step 1), the ribose residue is then removed by methylxanthosine nucleosidase (FIG. 1, step 2). The ribose free 7-methyxanthosine undergoes a second methylation catalysed by MXMT to form 3,7-dimethylxanthine (theobromine) (FIG. 1, step 3), which is further methylated by DXMT to form 1,3,7-trimethylxanthine (caffeine) [Ogita, S., et al. (2005) Plant Biotechnology 22(5): 461-468].

Many groups have been researching caffeine biosynthesis in coffee in order to reduce caffeine accumulation within the plant. For example, the group of Ogita et al. [Ogita et al., Nature (2003) 423: 823; Ogita et al., Plant Molecular Biology (2004) 54(6): 931-941; and Ogita et al. (2005) supra], produced decaffeinated Arabica coffee plants through overexpression of a transgenic RNAi cassette. They designed their RNAi constructs to target the 3′-untranslated region (UTR) and the coding region of CaMXMT1. The overexpression of the CaMXMT1 RNAi constructs reduced the transcript levels of not only CaMXMT1 but also CaDXMT1 and CaXMT1. This is likely to be a result of the similarity shared between the coding regions of the methyltransferases (over 90%) where the primary small double-stranded RNAs (dsRNA) produces many secondary smaller dsRNA which target the mRNA sequences of CaXMT1 and CaDXMT1. Through this method they were able to reduce caffeine accumulation in the leaves by an average of 50% with one example exhibiting a 70% reduction.

Several patent applications relate to downregulation of genes involved in caffeine synthesis wherein downregulation is effected by ribozymes (U.S. Patent Application No. 2003/0014775) or by RNA interference (RNAi) using anti-sense molecules (U.S. Patent Application Nos. 2008/0127373 and 2002/0108143 and PCT publication No. WO 1998/036053).

Additional background art includes U.S. Patent Application No. 2017/0014449.

### SUMMARY OF THE INVENTION

According to an aspect of some embodiments of the present invention there is provided a coffee plant comprising a genome comprising a loss of function mutation in a nucleic acid sequence encoding at least one component of a caffeine biosynthesis pathway.

According to an aspect of some embodiments of the present invention there is provided a method of producing a coffee plant or part thereof, the method comprising: (a) subjecting a coffee plant cell to a DNA editing agent directed at a nucleic acid sequence encoding at least one component of a caffeine biosynthesis pathway to result in a loss of function mutation in the nucleic acid sequence encoding the at least one component of the caffeine biosynthesis pathway; and (b) regenerating a coffee plant or part thereof from the coffee plant cell.

According to an aspect of some embodiments of the present invention there is provided a nucleic acid construct comprising a nucleic acid sequence encoding a DNA editing agent directed towards at least one component of a caffeine biosynthesis pathway being operably linked to a plant promoter for expressing the DNA editing agent in a cell of a coffee plant.

According to an aspect of some embodiments of the present invention there is provided a plant part of the coffee plant of some embodiments of the invention.

According to an aspect of some embodiments of the present invention there is provided a method of producing coffee beans with reduced caffeine content, the method comprising: (a) growing the plant of some embodiments of the invention; and (b) harvesting beans from the plant.

According to an aspect of some embodiments of the present invention there is provided a method of producing coffee with reduced caffeine content, the method comprising subjecting beans of some embodiments of the invention to extraction, dehydration and optionally roasting.

According to an aspect of some embodiments of the present invention there is provided coffee of the beans of some embodiments of the invention.

According to an aspect of some embodiments of the present invention there is provided coffee of the beans produced by the method of some embodiments of the invention.

According to some embodiments of the invention, the method further comprises harvesting beans from the coffee plant.

According to some embodiments of the invention, the method further comprises selfing or crossing the coffee plant.

According to some embodiments of the invention, the mutation occurs in at least one allele.

According to some embodiments of the invention, the mutation occurs in all alleles.

According to some embodiments of the invention, the coffee plant or progeny thereof of some embodiments of the invention, having been treated with a DNA editing agent directed to the nucleic acid sequence encoding the at least one component of the caffeine biosynthesis pathway.

According to some embodiments of the invention, the mutation is selected from the group consisting of a deletion, an insertion, an insertion/deletion (Indel), and a substitution.

According to some embodiments of the invention, the coffee plant is from a species Coffea canephora.

According to some embodiments of the invention, the coffee plant is from a species Coffea arabica.

According to some embodiments of the invention, the subjecting is to a nucleic acid construct encoding the DNA editing agent.

According to some embodiments of the invention, the subjecting is by a DNA-free delivery method.

According to some embodiments of the invention, the coffee plant comprises at least 5% reduction in caffeine as compared to that of a coffee plant of the same genetic background and developmental stage and growth conditions devoid of the loss of function mutation.

According to some embodiments of the invention, the DNA editing agent is a non-integrated DNA editing agent.

According to some embodiments of the invention, the DNA editing agent comprises at least one sgRNA.

According to some embodiments of the invention, the sgRNA comprises a nucleic acid sequence selected from the group consisting of SEQ ID NOs: 51-78.

According to some embodiments of the invention, the DNA editing agent does not comprise an endonuclease.

According to some embodiments of the invention, the DNA editing agent comprises an endonuclease.

According to some embodiments of the invention, the DNA editing agent is of a DNA editing system selected from the group consisting of meganucleases, Zinc finger nucleases (ZFNs), transcription-activator like effector nucleases (TALENs), CRISPR-endonuclease, dCRISPR-endonuclease, and a homing endonuclease.

According to some embodiments of the invention, the DNA editing agent is of a DNA editing system comprising CRISPR-Cas.

According to some embodiments of the invention, the DNA editing agent is linked to a reporter for monitoring expression in a cell.

According to some embodiments of the invention, the reporter is a fluorescent protein.

According to some embodiments of the invention, the DNA editing agent is directed to a nucleic sequence that is at least 90% identical between Cc09_g06970 (set forth in SEQ ID NO: 9), Cc09_g06960 (set forth in SEQ ID NO: 7), Cc00_g24720 (set forth in SEQ ID NO: 1), Cc09_g06950 (set forth in SEQ ID NO: 5), Cc01_g00720 (set forth in SEQ ID NO: 3) and Cc02_g09350 (set forth in SEQ ID NO: 11).

According to some embodiments of the invention, the DNA editing agent is directed to a nucleic acid segment comprised in a nucleic acid sequence as set forth in any one of SEQ ID NOs: 26-31, 33-36, 38-41, 43-45, 47-48 or 50.

According to some embodiments of the invention, the at least one component of a caffeine biosynthesis pathway is a methyltransferase.

According to some embodiments of the invention, the methyltransferase comprises a core SAM-binding domain.

According to some embodiments of the invention, the methyltransferase is a N-methyltransferase.

According to some embodiments of the invention, the N-methyltransferase is selected from the group consisting of a xanthosine methyltransferase (XMT), a 7-methyxanthine methyltrasferase (MXMT), and 3,7-dimethylxanthine methyltransferase (DXMT).

According to some embodiments of the invention, the N-methyltransferase is selected from the group consisting of Cc09_g06970 (set forth in SEQ ID NO: 10), Cc09_g06960 (set forth in SEQ ID NO: 8), Cc00_g24720 (set forth in SEQ ID NO: 2), Cc09_g06950 (set forth in SEQ ID NO: 6), Cc01_g00720 (set forth in SEQ ID NO: 4), Cc02_g09350 (set forth in SEQ ID NO: 12), BAC75663.1 (set forth in SEQ ID NO: 14), ABD90686.1 (set forth in SEQ ID NO: 16), BAB39215.1 (set forth in SEQ ID NO: 18), ABD90685.1 (set forth in SEQ ID NO: 20), BAB39216.1 (set forth in SEQ ID NO: 22), and BAC75664.1 (set forth in SEQ ID NO: 24).

According to some embodiments of the invention, the coffee plant is non-transgenic.

According to some embodiments of the invention, the plant part being a bean.

According to some embodiments of the invention, the bean is dry.

According to some embodiments of the invention, the coffee being in a powder form.

According to some embodiments of the invention, the coffee being in a granulated form.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

### BRIEF DESCRIPTION OF THE SEVERAL VIEWS OF THE DRAWINGS

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIG. 1 shows the caffeine biosynthetic pathway in coffee plants. The first (1), third (3), and fourth (4) steps feature methyl group transfer, and the second step (2) involves ribose removal. XMT, xanthosine methyltransferase; MXMT, 7-methylxanthine methyltransferase; DXMT, 3,7-dimethylxanthine methyltransferase. Incorporated and modified from Ogita, S., et al. (2005) Plant Biotechnology 22(5): 461-468.

FIG. 2 shows a protein alignment of the selected candidate genes from Coffea canephora (C. canephora) and the characterized methyltransferases from Coffea arabica (C. arabica) involved in the biosynthesis of caffeine (as set forth in SEQ ID NOs: 2, 4, 6, 8, 10, 14, 18, 22 and 24).

FIG. 3 shows neighbor-joining analysis showing the evolutionary relationship of N-methyltransferases sequences from 10 plant species. The optimal tree with the sum of branch length=76.09435312 is shown. The tree was calculated in MEGA v6 from an amino acid alignment (MUSCLE). The percentage of replicate trees in which the associated taxa clustered together in the bootstrap test (100 replicates) are shown as colored branches (red <40%; green >80%). Gene IDs in red-bold indicate the genes from C. arabica that have been characterized in the caffeine biosynthesis pathway and that were used as query sequences to retrieve closely-related genes in the genome of C. canephora. Gene IDs in green-bold show C. canephora candidate genes that are the most likely closest homologs to the C. arabica genes involved in caffeine biosynthesis. All other gene IDs in green correspond to other C. canephora N-methyltransferases retrieved.

FIG. 4 shows gene expression of selected candidate genes in C. canephora tissues. The closest homologs of XMT, MXMT and DXMT (i.e. Cc09_g06970, Cc00_g24270 and Cc01_g00720, respectively) are moderately to highly expressed in different leaf tissues. Data was retrieved from www(dot)coffee-genome(dot)org/ and the detailed description of the RNA-seq data used for gene expression analysis may be found in Denoued et al., Science (2014) 345(6201):1181-1184.

FIG. 5 shows gene expression of selected candidate genes in C. canephora tissues. Additional homologs of XMT, MXMT and DXMT (i.e. Cc09_g06960 and Cc09_g06950) have low or moderate expression in different leaf tissues. Data was retrieved from www(dot)coffee-genome(dot)org/ and the detailed description of the RNA-seq data used for gene expression analysis can be found in Denoued et al., 2014, supra.

FIG. 6 shows multiple alignment of the 5 selected candidate genes identified in the coffee genome as putative homologs of the characterized N-methyltransferases which are reported to be involved in the biosynthesis of caffeine (as set forth in SEQ ID NOs: 1, 3, 5, 7 and 9). The nucleotide sequences were aligned with MUSCLE using default parameters. The target sites of sgRNAs 6, 7, 11, 12, 13, 14, 37 and 38 are marked on the candidate genes in red characters or highlighted in turquoise if there are overlapping sequences with other sgRNAs (e.g. sgRNA 11 and sgRNA 37). The PAM region is highlighted in grey.

FIGS. 7A-E show partial nucleotide sequences of the selected C. canephora genes, which were targeted with the listed sgRNAs. Bold characters illustrate allelic variation between the 4 lines of coffee examined; bold and underlined characters illustrate the sequence targeted by the sgRNAs; and underlined character illustrates the Protospacer Adjacent Motif (PAM) site. The target sgRNA sequences for each of sgRNAs 6, 7, 11, 12, 13 and 14 are provided below (of note, these sequences are not the sgRNA sequences used for transfection e.g. in plasmids), shaded illustrates the PAM site, these are listed in 5′ to 3′order. Sequences are set forth in SEQ ID NOs: 25-48.

FIGS. 8A-G show sequencing analysis and T7 assay revealing the presence of mutations in some of the selected candidate genes in chromosome 9 Cc09g06960 (xmt/mxmt/dxmt), and Cc09g06970 (xmt). (FIG. 8A) Image representing genes in chromosome 9 (Cc09g06950, Cc09g06960, and Cc09g06970) with a putative role in caffeine biosynthesis indicating the relative positions where the sgRNAs were designed and selected based on conserved regions with the other closely related N-methyltransferase genes Cc00g24720 and Cc01g00720. (FIG. 8B) Cc09g06950, Cc09g06960, and Cc09g06970 loci were amplified with specific primers outside of the sgRNAs region as indicated in FIG. 8A (P-23 to P-28) and cloned into pBLUNT (Invitrogen) for sequence analysis and T7E1 assay. (FIG. 8C) Mutations detection measured by the T7E1 assay. “27” indicates control plasmid without sgRNAs. “23” and “25” are the combination of the sgRNAs used. Red asterisks indicate positive evidence of gene-editing. (FIGS. 8D-E) Mutant DNA sequences induced by expression of the genome editing machinery guided by specific sgRNAs are aligned to the wild-type (2027-Ctrl) sequence. The PAM is indicated by a black line and the sgRNAs position in red rectangles. For gene Cc09g06960 1 base pair (bp) deletion (FIG. 8D) was found in 2 out of 7 clones analyzed (labeled as 2023-3 and 2023-6) and for gene Cc09g06970 1 bp insertion (FIG. 8E) was found in 2 out of 7 clones analyzed (labeled as 2023-3 and 2023-4). The sequences of the other 5 clones for each gene are shown and are identical to the wild type sequence. (FIGS. 8F-G) Additional mutant sequences for genes Cc09g06950 and Cc09g06970. A large deletion of 289 bp was found in gene Cc09g06950 by sequencing individual cloned amplicons (4 out of 8 clones) and a deletion of 210 bp and 40 bp re-arrangements were found in gene Cc09g06970 by sequencing individual cloned amplicons (3 out of 4 clones). sgRNA positions are indicated in red characters and the PAM region is highlighted in grey.

FIGS. 9A-F show regeneration of transfected coffee protoplasts for all traits. (FIG. 9A) Freshly isolated coffee protoplasts, which were subjected to transfection with plasmids pDK2027, pDK2023 or pDK2025; (FIG. 9B) First cell divisions occur 48 hours after protoplast isolation and transfection; (FIG. 9C) Embryogenic microcalli obtained from transfected protoplasts three months post-transfection; (FIG. 9D) Embryogenic calli of 1-2 mm develop from microcalli; (FIG. 9E) Globular and torpedo embryos regenerating from embryogenic calli; (FIG. 9F) Regenerated coffee plantlets.

FIG. 10 shows additional sgRNAs designed to target the candidate genes from C. canephora. Of note, PAM region is highlighted in grey.

### DESCRIPTION OF SPECIFIC EMBODIMENTS OF THE INVENTION

The present invention, in some embodiments thereof, relates to compositions and methods for reducing caffeine content in coffee beans.

The principles and operation of the present invention may be better understood with reference to the drawings and accompanying descriptions.

Before explaining at least one embodiment of the invention in detail, it is to be understood that the invention is not necessarily limited in its application to the details set forth in the following description or exemplified by the Examples. The invention is capable of other embodiments or of being practiced or carried out in various ways. Also, it is to be understood that the phraseology and terminology employed herein is for the purpose of description and should not be regarded as limiting.

Various post-harvest processes are currently employed to commercially produce decaffeinated coffee. Although much research and development has been performed to optimise these processes, they are unable to remove caffeine from the unroasted bean without affecting other components which contribute flavour to the final beverage.

Caffeine is a purine alkaloid, it is a secondary metabolite derived from purine metabolism. Xanthosine from purine metabolism undergoes three methylation steps and removal of a ribose residue to form caffeine. These methylation steps are attributed to three methyltransferases, xanthosine methyltransferase (XMT), 7-methyxanthine methyltrasferase (MXMT or theobromine synthase), and 3,7-dimethylxanthine methyltransferase (DXMT or caffeine synthase). Creation of coffee plants with reduced caffeine accumulation will result in a product which will require less post-harvest processing and improved beverage characteristics.

Genome editing is now an established method which can be used to target specific sequences of genomic DNA for modification. A modification of just a few nucleotides within the coding region of a gene can frequently result in the disruption of the translation of mRNA to protein which renders the resulting protein inactive. This kind of gene knockout can be used to modify key enzymes in metabolic pathways to reduce the accumulation of specific secondary metabolites like caffeine.

While reducing the present invention to practice, the present inventors have devised a gene editing technology designed to target and interfere with caffeine synthesis in coffee plants. The technology described herein targets endogenous methyltransferases involved in caffeine synthesis, e.g. XMT, MXMT and DXMT, by introducing mutations which cause loss of function mutations and downregulate caffeine biosynthesis. Moreover, the gene technology described does not necessitate the classical molecular genetic and transgenic tools comprising expression cassettes that have a promoter, terminator, selection marker.

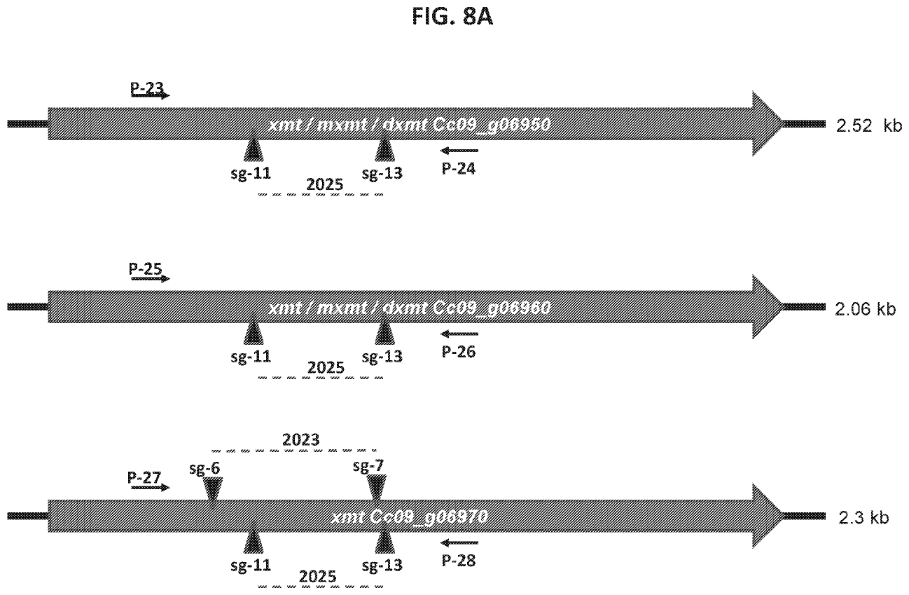

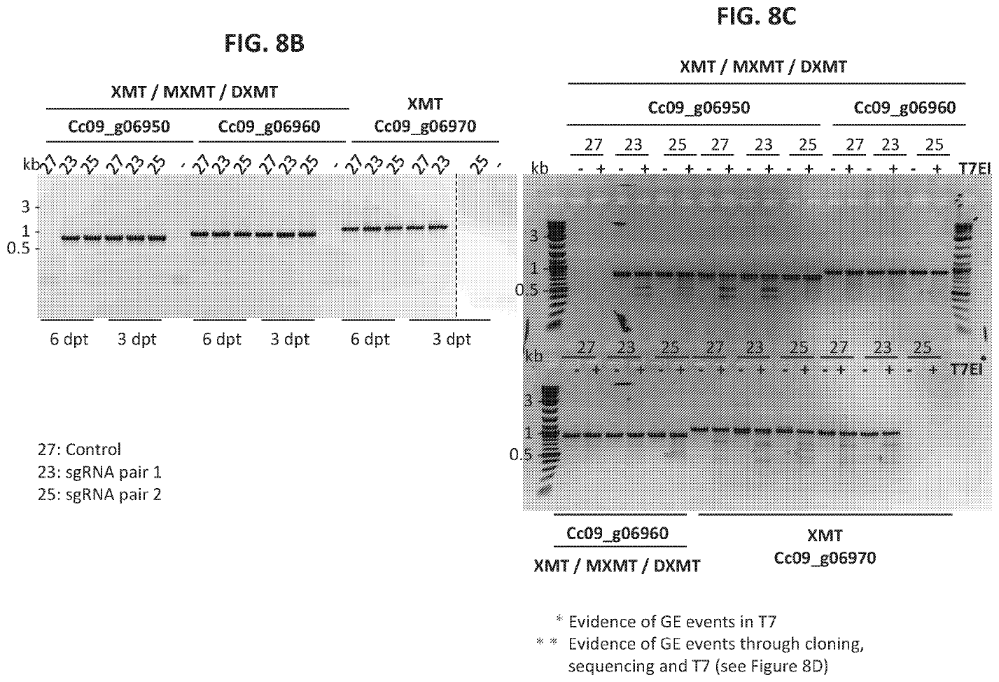

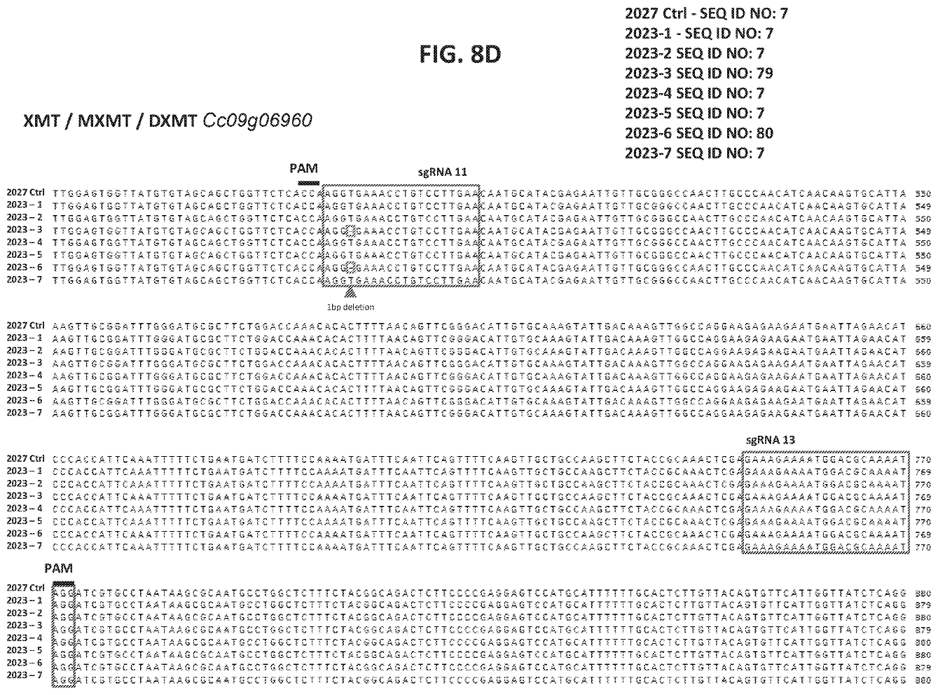

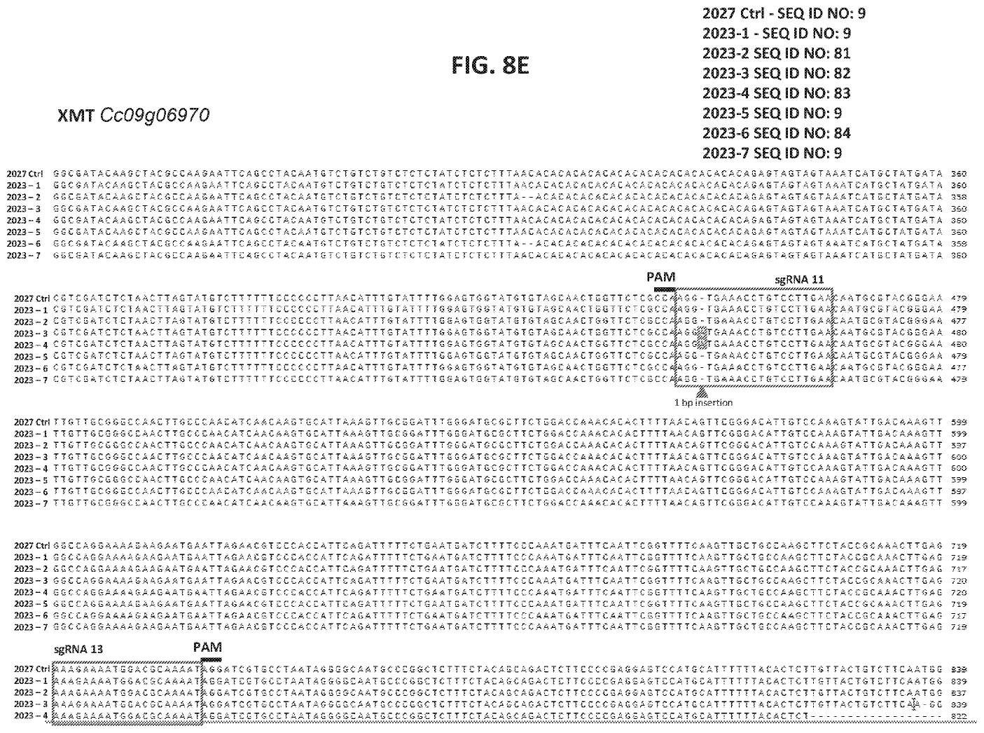

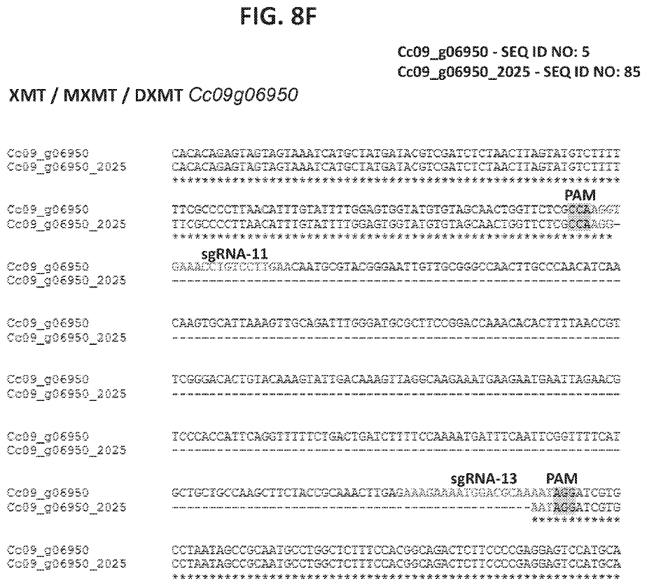

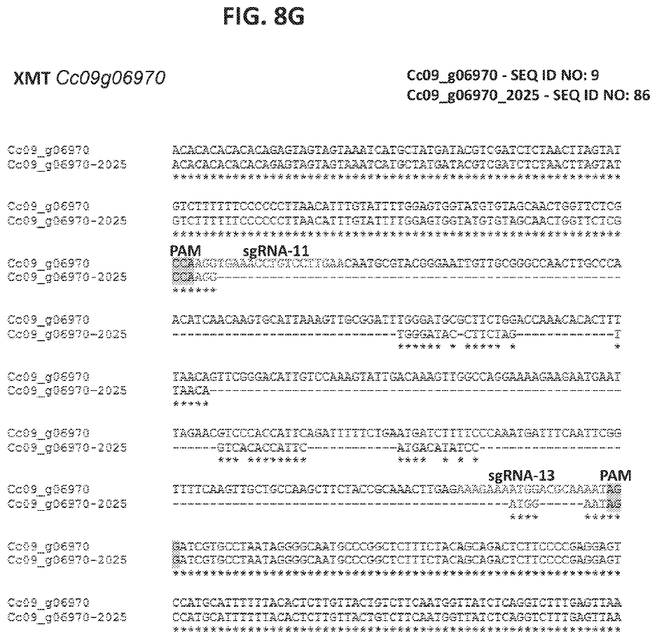

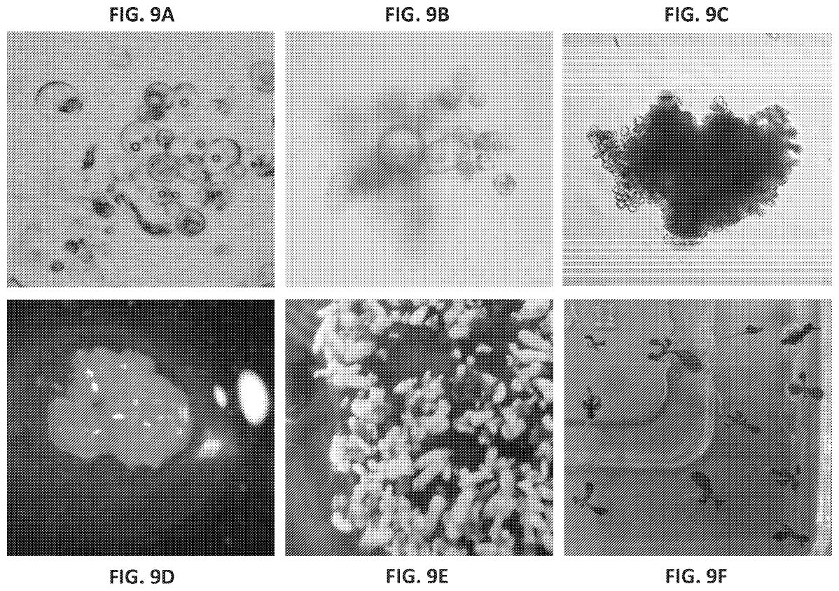

As is shown herein below and in the Examples section which follows, the present inventors have identified caffeine biosynthesis genes which can be targeted to reduce caffeine production in coffee plants (see Example 1, below). The present inventors then designed sgRNAs which target XMT, MXMT and DXMT genes and can be used in a CRISPR/Cas9 system to target at least one of these methyltransferases (see Example 2, below). XMT, MXMT and DXMT genes were targeted with two pairs of sgRNAs in coffee protoplasts, and precise mutations were induced as evident by sequencing analysis and T7 assay (see FIGS. 8B-E and Example 2, below). Next, coffee plants were regenerated from the protoplasts which underwent the genome-editing events (see FIGS. 9A-F and Example 3, below). Taken together, this technology can be used to generate coffee plants and consequently coffee beans comprising reduced caffeine content without affecting other components which contribute flavour.

Thus, according to one aspect of the present invention there is provided a method of producing a coffee plant or part thereof, the method comprising: (a) subjecting a coffee plant cell to a DNA editing agent directed at a nucleic acid sequence encoding at least one component of a caffeine biosynthesis pathway to result in a loss of function mutation in the nucleic acid sequence encoding the at least one component of the caffeine biosynthesis pathway; and (b) regenerating a coffee plant or part thereof from the coffee plant cell.

As used herein a “coffee” refers to a plant of the family Rubiaceae, genus Coffea. There are many coffee species. Embodiments of the invention may refer to two primary commercial coffee species: Coffea Arabica (C. arabica), which is known as arabica coffee, and Coffea canephora, which is known as robusta coffee (C. robusta). Coffea liberica Bull. ex Hiern is also contemplated here which makes up 3% of the world coffee bean market. Also known as Coffea arnoldiana De Wild or more commonly as Liberian coffee. Coffees from the species Arabica are also generally called “Brazils” or they are classified as “other milds”. Brazilian coffees come from Brazil and “other milds” are grown in other high-grade coffee producing countries, which are generally recognized as including Colombia, Guatemala, Sumatra, Indonesia, Costa Rica, Mexico, United States (Hawaii), El Salvador, Peru, Kenya, Ethiopia and Jamaica. Coffea canephora, i.e. robusta, is typically used as a low-cost extender for arabica coffees. These robusta coffees are typically grown in the lower regions of West and Central Africa, India, Southeast Asia, Indonesia, and also Brazil. A person skilled in the art will appreciate that a geographical area refers to a coffee growing region where the coffee growing process utilizes identical coffee seedlings and where the growing environment is similar.

As used herein “plant” refers to whole plant(s), a grafted plant, ancestors and progeny of the plants and plant parts, including seeds, fruits, shoots, stems, roots (including tubers), rootstock, scion, and plant cells, tissues and organs.

According to a specific embodiment, the plant is a plant cell e.g., plant cell in an embryonic cell suspension.

According to a specific embodiment, the plant part is a bean.

“Grain,” “seed,” or “bean,” refers to a flowering plant's unit of reproduction, capable of developing into another such plant. As used herein, especially with respect to coffee plants, the terms are used synonymously and interchangeably.

According to a specific embodiment, the cell is a germ cell.

According to a specific embodiment, the plant cell is an embryogenic cell.

According to a specific embodiment, the cell is a somatic cell.

According to a specific embodiment, the plant cell is a somatic embryogenic cell.

According to a specific embodiment, the cell is a protoplast.

According to one embodiment, the protoplast is derived from any plant tissue e.g., fruit, flowers, roots, leaves, embryonic cell suspension, calli or seedling tissue.

The plant may be in any form including suspension cultures, protoplasts, embryos, meristematic regions, callus tissue, leaves, gametophytes, sporophytes, pollen, and microspores.

According to a specific embodiment, the plant part comprises DNA.

According to a specific embodiment, the coffee plant is of a coffee breeding line, more preferably an elite line.

According to a specific embodiment, the coffee plant is of an elite line.

According to a specific embodiment, the coffee plant is of a purebred line.

According to a specific embodiment, the coffee plant is of a coffee variety or breeding germplasm.

The term “breeding line”, as used herein, refers to a line of a cultivated coffee having commercially valuable or agronomically desirable characteristics, as opposed to wild varieties or landraces. The term includes reference to an elite breeding line or elite line, which represents an essentially homozygous, usually inbred, line of plants used to produce commercial F₁ hybrids. An elite breeding line is obtained by breeding and selection for superior agronomic performance comprising a multitude of agronomically desirable traits. An elite plant is any plant from an elite line. Superior agronomic performance refers to a desired combination of agronomically desirable traits as defined herein, wherein it is desirable that the majority, preferably all of the agronomically desirable traits are improved in the elite breeding line as compared to a non-elite breeding line. Elite breeding lines are essentially homozygous and are preferably inbred lines.

The term “elite line”, as used herein, refers to any line that has resulted from breeding and selection for superior agronomic performance. An elite line preferably is a line that has multiple, preferably at least 3, 4 5, 6 or more (genes for) desirable agronomic traits as defined herein.

The terms “cultivar” and “variety” are used interchangeable herein and denote a plant with has deliberately been developed by breeding, e.g., crossing and selection, for the purpose of being commercialized, e.g., used by farmers and growers, to produce agricultural products for own consumption or for commercialization. The term “breeding germplasm” denotes a plant having a biological status other than a “wild” status, which “wild” status indicates the original non-cultivated, or natural state of a plant or accession.

The term “breeding germplasm” includes, but is not limited to, semi-natural, semi-wild, weedy, traditional cultivar, landrace, breeding material, research material, breeder's line, synthetic population, hybrid, founder stock/base population, inbred line (parent of hybrid cultivar), segregating population, mutant/genetic stock, market class and advanced/improved cultivar. As used herein, the terms “purebred”, “pure inbred” or “inbred” are interchangeable and refer to a substantially homozygous plant or plant line obtained by repeated selfing and-or backcrossing.

A non-comprehensive list, of coffee varieties is provided hereinbelow:

Wild Coffee: This is the common name of “Coffea racemosa Lour” which is a coffee species native to Ethiopia.

Baron Goto Red: A coffee bean cultivar that is very similar to ‘Catuai Red’. It is grown at several sites in Hawaii.

Blue Mountain: Coffea arabica L. ‘Blue Mountain’. Also known commonly as Jamaican coffea or Kenyan coffea. It is a famous Arabica cultivar that originated in Jamaica but is now grown in Hawaii, PNG and Kenya. It is a superb coffee with a high quality cup flavor. It is characterized by a nutty aroma, bright acidity and a unique beef-bullion like flavor.

Bourbon: Coffea arabica L. ‘Bourbon’. A botanical variety or cultivar of Coffea Arabica which was first cultivated on the French controlled island of Bourbon, now called Reunion, located east of Madagascar in the Indian ocean.

Brazilian Coffea: Coffea arabica L. ‘Mundo Novo’. The common name used to identify the coffee plant cross created from the “Bourbon” and “Typica” varieties.

Caracol/Caracoli: Taken from the Spanish word Caracolillo meaning ‘seashell’ and describes the peaberry coffee bean.

Catimor: Is a coffee bean cultivar cross-developed between the strains of Caturra and Hibrido de Timor in Portugal in 1959. It is resistant to coffee leaf rust (Hemileia vastatrix). Newer cultivar selection with excellent yield but average quality.

Catuai: Is a cross between the Mundo Novo and the Caturra Arabica cultivars. Known for its high yield and is characterized by either yellow (Coffea arabica L. ‘Catuai Amarelo’) or red cherries (Coffea arabica L. ‘Catuai Vermelho’).

Caturra: A relatively recently developed sub-variety of the Coffea Arabica species that generally matures more quickly, gives greater yields, and is more disease resistant than the traditional “old Arabica” varieties like Bourbon and Typica.

Columbiana: A cultivar originating in Columbia. It is vigorous, heavy producer but average cup quality.

Congencis: Coffea Congencis—Coffee bean cultivar from the banks of Congo, it produces a good quality coffee but it is of low yield. Not suitable for commercial cultivation

Dewevreilt: Coffea Dewevreilt. A coffee bean cultivar discovered growing naturally in the forests of the Belgian Congo. Not considered suitable for commercial cultivation.

DybowskiiIt: Coffea DybowskiiIt. This coffee bean cultivar comes from the group of Eucoffea of inter-tropical Africa. Not considered suitable for commercial cultivation

Excelsa: Coffea Excelsa—A coffee bean cultivar discovered in 1904. Possesses natural resistance to diseases and delivers a high yield. Once aged it can deliver an odorous and pleasant taste, similar to var. Arabica.

Guadalupe: A cultivar of Coffea Arabica that is currently being evaluated in Hawaii.

Guatemala(n): A cultivar of Coffea Arabica that is being evaluated in other parts of Hawaii.

Hibrido de Timor: This is a cultivar that is a natural hybrid of Arabica and Robusta. It resembles Arabica coffee in that it has 44 chromosomes.

Icatu: A cultivar which mixes the “Arabica & Robusta hybrids” to the Arabica cultivars of Mundo Novo and Caturra.

Interspecific Hybrids: Hybrids of the coffee plant species and include; ICATU (Brazil; cross of Bourbon/MN & Robusta), S2828 (India; cross of Arabica & Liberia), Arabusta (Ivory Coast; cross of Arabica & Robusta).

‘K7’, ‘SL6’, ‘SL26’, ‘H66”, ‘KP532’: Promising new cultivars that are more resistant to the different variants of coffee plant disease like Hemileia.

Kent: A cultivar of the Arabica coffee bean that was originally developed in Mysore India and grown in East Africa. It is a high yielding plant that is resistant to the “coffee rust” decease but is very susceptible to coffee berry disease. It is being replaced gradually by the more resistant cultivar's of ‘S.288’, ‘S.333’ and ‘S.795’.

Kouillou: Name of a Coffea canephora (Robusta) variety whose name comes from a river in Gabon in Madagascar.

Laurina: A drought resistant cultivar possessing a good quality cup but with only fair yields.

Maragogipe/Maragogype: Coffea arabica L. ‘Maragopipe’. Also known as “Elephant Bean”. A mutant variety of Coffea Arabica (Typica) which was first discovered (1884) in Maragogype County in the Bahia state of Brazil.

Mauritiana: Coffea Mauritiana. A coffee bean cultivar that creates a bitter cup. Not considered suitable for commercial cultivation.

Mundo Novo: A natural hybrid originating in Brazil as a cross between the varieties of ‘Arabica’ and ‘Bourbon’. It is a very vigorous plant that grows well at 3,500 to 5,500 feet (1,070 m to 1,525 m), is resistant to disease and has a high production yield. Tends to mature later than other cultivars.

Neo-Arnoldiana: Coffea Neo-Arnoldiana is a coffee bean cultivar that is grown in some parts of the Congo because of its high yield. It is not considered suitable for commercial cultivation.

Nganda: Coffea canephora Pierre ex A. Froehner ‘Nganda’. Where the upright form of the coffee plant Coffea Canephora is called Robusta its spreading version is also known as Nganda or Kouillou.

Paca: Created by El Salvador's agricultural scientists, this cultivar of Arabica is shorter and higher yielding than Bourbon but many believe it to be of an inferior cup in spite of its popularity in Latin America.

Pacamara: An Arabica cultivar created by crossing the low yield large bean variety Maragogipe with the higher yielding Paca. Developed in El Salvador in the 1960's this bean is about 75% larger than the average coffee bean.

Pache Colis: An Arabica cultivar being a cross between the cultivars Caturra and Pache comum. Originally found growing on a Guatemala farm in Mataquescuintla.

Pache Comum: A cultivar mutation of Typica (Arabica) developed in Santa Rosa Guatemala. It adapts well and is noted for its smooth and somewhat flat cup.

Preanger: A coffee plant cultivar currently being evaluated in Hawaii.

Pretoria: A coffee plant cultivar currently being evaluated in Hawaii.

Purpurescens: A coffee plant cultivar that is characterized by its unusual purple leaves.

Racemosa: Coffea Racemosa—A coffee bean cultivar that looses its leaves during the dry season and re-grows them at the start of the rainy season. It is generally rated as poor tasting and not suitable for commercial cultivation.

Ruiru 11: Is a new dwarf hybrid which was developed at the Coffee Research Station at Ruiru in Kenya and launched on to the market in 1985. Ruiru 11 is resistant to both coffee berry disease and to coffee leaf rust. It is also high yielding and suitable for planting at twice the normal density.

San Ramon: Coffea arabica L. ‘San Ramon’. It is a dwarf variety of Arabica var typica. A small stature tree that is wind tolerant, high yield and drought resistant.

Tico: A cultivar of Coffea Arabica grown in Central America.

Timor Hybrid: A variety of coffee tree that was found in Timor in 1940s and is a natural occurring cross between the Arabica and Robusta species.

Typica: The correct botanical name is Coffea arabica L. ‘Typica’. It is a coffee variety of Coffea Arabica that is native to Ethiopia. Var Typica is the oldest and most well-known of all the coffee varieties and still constitutes the bulk of the world's coffee production. Some of the best Latin-American coffees are from the Typica stock. The limits of its low yield production are made up for in its excellent cup.

According to a specific embodiment, the coffee plant is from the species Coffea canephora.

According to a specific embodiment, the coffee plant is from the species Coffea arabica.

According to a specific embodiment, the coffee plant is from the species Arabusta.

According to a specific embodiment, the coffee plant is from the species Liberica.

As used herein, the term “caffeine” refers to the xanthine alkaloid 1,3,7-Trimethylxanthine.

Caffeine is a secondary metabolite derived from purine metabolism. The main caffeine biosynthetic pathway is a sequence consisting of xanthosine→7-methylxanthosine→7-methylxanthine→theobromine→caffeine, wherein the biosynthesis of caffeine includes three methylation steps and removal of a ribose residue to form caffeine. The methylation steps are attributed to methyltransferases.

According to a specific embodiment, the methyltransferases in the caffeine biosynthesis pathway are S-Adenosyl methionine (SAM)-dependent methyltransferases.

According to a specific embodiment, the methyltransferases in the caffeine biosynthesis pathway are N-methyltransferases.

According to a specific embodiment, the methyltransferases in the caffeine biosynthesis pathway are XMT, MXMT and DXMT.

As used herein, the terms “XMT” or “xanthosine methyltransferase” refer to an enzyme as set forth in EC 2.1.1.158. Typically XMT catalyzes the transfer of a methyl group to xanthosine to form 7-methylxanthosine.

According to a specific embodiment, the XMT enzyme is encoded from the C. Canephora gene Cc09_g06970.

According to a specific embodiment, the XMT enzyme is encoded from the Coffea arabica gene AB048793.

According to a specific embodiment, the XMT enzyme is encoded from the Coffea canephora gene DQ422954.

As used herein, the terms “MXMT” or “7-methyxanthine methyltrasferase” refer to an enzyme as set forth in EC 2.1.1.159 (also referred to as theobromine synthase). Typically, MXMT catalyzes the transfer of a methyl group to 7-methylxanthine to form 3,7-dimethylxanthine (theobromine).

According to a specific embodiment, the MXMT enzyme is encoded from the C. Canephora gene Cc00_g24720.

According to a specific embodiment, the MXMT enzyme is encoded from the Coffea arabica gene AB048794.1.

According to a specific embodiment, the MXMT enzyme is encoded from the Coffea arabica gene AB084126.

As used herein, the terms “DXMT” or “3,7-dimethylxanthine methyltransferase” refer to an enzyme as set forth in EC 2.1.1.160 (also referred to as caffeine synthase). Typically, DXMT catalyzes the transfer of a methyl group to 3,7-dimethylxanthine (theobromine) to form 1,3,7-trimethylxanthine (caffeine).

According to a specific embodiment, the DXMT enzyme is encoded from the C. Canephora genes Cc01_g00720 or Cc02_g09350.

According to a specific embodiment, the DXMT enzyme is encoded from the C. Canephora gene DQ422955.

According to a specific embodiment, the DXMT enzyme is encoded from the Coffea arabica gene AB084125.1.

According to a specific embodiment, the N-methyltransferase (e.g. XMT/MXMT/DXMT gene) is encoded from the C. Canephora genes Cc09_g06950 or Cc09_g06960.

According to one embodiment, the coffee plant of some embodiments of the invention comprises a loss of function mutation in the nucleic acid sequence encoding at least one component of the caffeine biosynthesis pathway.

As used herein “loss of function” mutation refers to a genomic aberration which results in reduced ability (i.e., impaired function) or inability of a methyltransferase (e.g. XMT, MXMT and/or DXMT) to synthesize caffeine from xanthosine. As used herein “reduced ability” refers to reduced methyltransferase activity (i.e., caffeine biosynthesis) as compared to that of the wild-type enzyme devoid of the loss of function mutation. According to a specific embodiment, the reduced activity is by at least about 5%, 10%, 20%, 30%, 40%, 50%, 60%, 70%, 80%, 90% or even more as compared to that of the wild-type enzyme under the same assay conditions. Methyltransferase activity can be detected by ELISA assay (commercially available from Abcam and Enzo Life Sciences).

According to a specific embodiment, the loss of function mutation results in no expression of the methyltransferase (e.g. XMT, MXMT and/or DXMT) mRNA or protein.

According to a specific embodiment, the loss of function mutation results in expression of a methyltransferase (e.g. XMT, MXMT and/or DXMT) protein which is not capable of supporting caffeine biosynthesis.

According to one embodiment, the coffee plant of some embodiments of the invention comprises a loss of function mutation in a nucleic acid sequence encoding one, two, three, four or more components of the caffeine biosynthesis pathway.

According to one embodiment, the coffee plant of some embodiments of the invention comprises a loss of function mutation in a nucleic acid sequence encoding XMT.

According to one embodiment, the coffee plant of some embodiments of the invention comprises a loss of function mutation in a nucleic acid sequence encoding MXMT.

According to one embodiment, the coffee plant of some embodiments of the invention comprises a loss of function mutation in a nucleic acid sequence encoding DXMT.

According to one embodiment, the coffee plant of some embodiments of the invention comprises a loss of function mutation in a nucleic acid sequence encoding any two of XMT, MXMT or DXMT.

According to one embodiment, the coffee plant of some embodiments of the invention comprises a loss of function mutation in a nucleic acid sequence encoding all of XMT, MXMT and DXMT.

According to a specific embodiment, the loss of function mutation is selected from the group consisting of a deletion, insertion, insertion-deletion (Indel), inversion, substitution and a combination of same (e.g., deletion and substitution e.g., deletions and SNPs).

According to a specific embodiment, the mutation is homozygous.

According to a specific embodiment, the mutation is heterozygous.

Examples of suggested target positions for generation of loss of function mutations are provided in SEQ ID Nos: 26-50.

In order to induce a loss of function mutation in a nucleic acid sequence encoding at least one component of the caffeine biosynthesis pathway, a DNA editing agent is utilized.

Following is a description of various non-limiting examples of methods and DNA editing agents used to introduce nucleic acid alterations to a gene of interest and agents for implementing same that can be used according to specific embodiments of the present disclosure.

Genome Editing using engineered endonucleases—this approach refers to a reverse genetics method using artificially engineered nucleases to typically cut and create specific double-stranded breaks at a desired location(s) in the genome, which are then repaired by cellular endogenous processes such as, homologous recombination (HR) or non-homologous end-joining (NHEJ). NHEJ directly joins the DNA ends in a double-stranded break, while HR utilizes a homologous donor sequence as a template (i.e the sister chromatid formed during S-phase) for regenerating the missing DNA sequence at the break site. In order to introduce specific nucleotide modifications to the genomic DNA, a donor DNA repair template containing the desired sequence must be present during HR (exogenously provided single stranded or double stranded DNA).

Genome editing cannot be performed using traditional restriction endonucleases since most restriction enzymes recognize a few base pairs on the DNA as their target and these sequences often will be found in many locations across the genome resulting in multiple cuts which are not limited to a desired location. To overcome this challenge and create site-specific single- or double-stranded breaks, several distinct classes of nucleases have been discovered and bioengineered to date. These include the meganucleases, Zinc finger nucleases (ZFNs), transcription-activator like effector nucleases (TALENs) and CRISPR/Cas system.

Meganucleases—Meganucleases are commonly grouped into four families: the LAGLIDADG family, the GIY-YIG family, the His-Cys box family and the HNH family. These families are characterized by structural motifs, which affect catalytic activity and recognition sequence. For instance, members of the LAGLIDADG family are characterized by having either one or two copies of the conserved LAGLIDADG motif. The four families of meganucleases are widely separated from one another with respect to conserved structural elements and, consequently, DNA recognition sequence specificity and catalytic activity. Meganucleases are found commonly in microbial species and have the unique property of having very long recognition sequences (>14 bp) thus making them naturally very specific for cutting at a desired location.

This can be exploited to make site-specific double-stranded breaks in genome editing. One of skill in the art can use these naturally occurring meganucleases, however the number of such naturally occurring meganucleases is limited. To overcome this challenge, mutagenesis and high throughput screening methods have been used to create meganuclease variants that recognize unique sequences. For example, various meganucleases have been fused to create hybrid enzymes that recognize a new sequence.

Alternatively, DNA interacting amino acids of the meganuclease can be altered to design sequence specific meganucleases (see e.g., U.S. Pat. No. 8,021,867). Meganucleases can be designed using the methods described in e.g., Certo, M T et al. Nature Methods (2012) 9:073-975; U.S. Pat. Nos. 8,304,222; 8,021,867; 8,119,381; 8,124,369; 8,129,134; 8,133,697; 8,143,015; 8,143,016; 8, 148,098; or 8, 163,514, the contents of each are incorporated herein by reference in their entirety. Alternatively, meganucleases with site specific cutting characteristics can be obtained using commercially available technologies e.g., Precision Biosciences' Directed Nuclease Editor™ genome editing technology.

ZFNs and TALENs—Two distinct classes of engineered nucleases, zinc-finger nucleases (ZFNs) and transcription activator-like effector nucleases (TALENs), have both proven to be effective at producing targeted double-stranded breaks (Christian et al., 2010; Kim et al., 1996; Li et al., 2011; Mahfouz et al., 2011; Miller et al., 2010).

Basically, ZFNs and TALENs restriction endonuclease technology utilizes a non-specific DNA cutting enzyme which is linked to a specific DNA binding domain (either a series of zinc finger domains or TALE repeats, respectively). Typically a restriction enzyme whose DNA recognition site and cleaving site are separate from each other is selected. The cleaving portion is separated and then linked to a DNA binding domain, thereby yielding an endonuclease with very high specificity for a desired sequence. An exemplary restriction enzyme with such properties is Fokl. Additionally Fokl has the advantage of requiring dimerization to have nuclease activity and this means the specificity increases dramatically as each nuclease partner recognizes a unique DNA sequence. To enhance this effect, Fokl nucleases have been engineered that can only function as heterodimers and have increased catalytic activity. The heterodimer functioning nucleases avoid the possibility of unwanted homodimer activity and thus increase specificity of the double-stranded break.

Thus, for example to target a specific site, ZFNs and TALENs are constructed as nuclease pairs, with each member of the pair designed to bind adjacent sequences at the targeted site. Upon transient expression in cells, the nucleases bind to their target sites and the Fokl domains heterodimerize to create a double-stranded break. Repair of these double-stranded breaks through the non-homologous end-joining (NHEJ) pathway often results in small deletions or small sequence insertions. Since each repair made by NHEJ is unique, the use of a single nuclease pair can produce an allelic series with a range of different deletions at the target site.

In general NHEJ is relatively accurate (about 85% of DSBs in human cells are repaired by NHEJ within about 30 min from detection) in gene editing erroneous NHEJ is relied upon as when the repair is accurate the nuclease will keep cutting until the repair product is mutagenic and the recognition/cut site/PAM motif is gone/mutated or that the transiently introduced nuclease is no longer present.

The deletions typically range anywhere from a few base pairs to a few hundred base pairs in length, but larger deletions have been successfully generated in cell culture by using two pairs of nucleases simultaneously (Carlson et al., 2012; Lee et al., 2010). In addition, when a fragment of DNA with homology to the targeted region is introduced in conjunction with the nuclease pair, the double-stranded break can be repaired via homologous recombination (HR) (e.g. in the presence of a donor template) to generate specific modifications (Li et al., 2011; Miller et al., 2010; Urnov et al., 2005).

Although the nuclease portions of both ZFNs and TALENs have similar properties, the difference between these engineered nucleases is in their DNA recognition peptide. ZFNs rely on Cys2-His2 zinc fingers and TALENs on TALEs. Both of these DNA recognizing peptide domains have the characteristic that they are naturally found in combinations in their proteins. Cys2-His2 Zinc fingers are typically found in repeats that are 3 bp apart and are found in diverse combinations in a variety of nucleic acid interacting proteins. TALEs on the other hand are found in repeats with a one-to-one recognition ratio between the amino acids and the recognized nucleotide pairs. Because both zinc fingers and TALEs happen in repeated patterns, different combinations can be tried to create a wide variety of sequence specificities. Approaches for making site-specific zinc finger endonucleases include, e.g., modular assembly (where Zinc fingers correlated with a triplet sequence are attached in a row to cover the required sequence), OPEN (low-stringency selection of peptide domains vs. triplet nucleotides followed by high-stringency selections of peptide combination vs. the final target in bacterial systems), and bacterial one-hybrid screening of zinc finger libraries, among others. ZFNs can also be designed and obtained commercially from e.g., Sangamo Biosciences™ (Richmond, Calif.).

Method for designing and obtaining TALENs are described in e.g. Reyon et al. Nature Biotechnology 2012 May; 30(5):460-5; Miller et al. Nat Biotechnol. (2011) 29: 143-148; Cermak et al. Nucleic Acids Research (2011) 39 (12): e82 and Zhang et al. Nature Biotechnology (2011) 29 (2): 149-53, incorporated herein by reference. A recently developed web-based program named Mojo Hand was introduced by Mayo Clinic for designing TAL and TALEN constructs for genome editing applications (can be accessed through www(dot)talendesign(dot)org). TALEN can also be designed and obtained commercially from e.g., Sangamo Biosciences™ (Richmond, Calif.).

T-GEE system (TargetGene's Genome Editing Engine)—A programmable nucleoprotein molecular complex containing a polypeptide moiety and a specificity conferring nucleic acid (SCNA) which assembles in-vivo, in a target cell, and is capable of interacting with the predetermined target nucleic acid sequence is provided. The programmable nucleoprotein molecular complex is capable of specifically modifying and/or editing a target site within the target nucleic acid sequence and/or modifying the function of the target nucleic acid sequence. Nucleoprotein composition comprises (a) polynucleotide molecule encoding a chimeric polypeptide and comprising (i) a functional domain capable of modifying the target site, and (ii) a linking domain that is capable of interacting with a specificity conferring nucleic acid, and (b) specificity conferring nucleic acid (SCNA) comprising (i) a nucleotide sequence complementary to a region of the target nucleic acid flanking the target site, and (ii) a recognition region capable of specifically attaching to the linking domain of the polypeptide. The composition enables modifying a predetermined nucleic acid sequence target precisely, reliably and cost-effectively with high specificity and binding capabilities of molecular complex to the target nucleic acid through base-pairing of specificity-conferring nucleic acid and a target nucleic acid. The composition is less genotoxic, modular in their assembly, utilize single platform without customization, practical for independent use outside of specialized core-facilities, and has shorter development time frame and reduced costs.

CRISPR-Cas system (also referred to herein as “CRISPR”)—Many bacteria and archea contain endogenous RNA-based adaptive immune systems that can degrade nucleic acids of invading phages and plasmids. These systems consist of clustered regularly interspaced short palindromic repeat (CRISPR) nucleotide sequences that produce RNA components and CRISPR associated (Cas) genes that encode protein components. The CRISPR RNAs (crRNAs) contain short stretches of homology to the DNA of specific viruses and plasmids and act as guides to direct Cas nucleases to degrade the complementary nucleic acids of the corresponding pathogen. Studies of the type II CRISPR/Cas system of Streptococcus pyogenes have shown that three components form a RNA/protein complex and together are sufficient for sequence-specific nuclease activity: the Cas9 nuclease, a crRNA containing 20 base pairs of homology to the target sequence, and a trans-activating crRNA (tracrRNA) (Jinek et al. Science (2012) 337: 816-821).

It was further demonstrated that a synthetic chimeric guide RNA (sgRNA) composed of a fusion between crRNA and tracrRNA could direct Cas9 to cleave DNA targets that are complementary to the crRNA in vitro. It was also demonstrated that transient expression of Cas9 in conjunction with synthetic sgRNAs can be used to produce targeted double-stranded brakes (DSBs) in a variety of different species (Cho et al., 2013, Nature Biotechnology 31, 230-232; Cong et al., 2013, Science 339: 819-823; DiCarlo et al., 2013, Nucleic Acids Research, 41: 4336-4343; Hwang et al., 2013, Nature Biotechnology 31: 227-229; Jinek et al., 2013, eLife 2013; 2:e00471; Mali et al., 2013, Science 339: 823-826).

The CRIPSR/Cas system for genome editing contains two distinct components: a sgRNA and an endonuclease e.g. Cas9.

The sgRNA is typically a 20-nucleotide sequence encoding a combination of the target homologous sequence (crRNA) and the endogenous bacterial RNA that links the crRNA to the Cas9 nuclease (tracrRNA) in a single chimeric transcript. The sgRNA/Cas9 complex is recruited to the target sequence by the base-pairing between the sgRNA sequence and the complement genomic DNA. For successful binding of Cas9, the genomic target sequence must also contain the correct Protospacer Adjacent Motif (PAM) sequence immediately following the target sequence. The binding of the sgRNA/Cas9 complex localizes the Cas9 to the genomic target sequence so that the Cas9 can cut both strands of the DNA causing a double-strand break (DSB). Just as with ZFNs and TALENs, the double-stranded breaks (DSBs) produced by CRISPR/Cas can undergo homologous recombination (HR) or non-homologous end joining (NHEJ) and are susceptible to specific sequence modification during DNA repair.

The Cas9 nuclease has two functional domains: RuvC and HNH, each cutting a different DNA strand. When both of these domains are active, the Cas9 causes double strand breaks (DSBs) in the genomic DNA.

A significant advantage of CRISPR/Cas is that the high efficiency of this system is coupled with the ability to easily create synthetic sgRNAs. This creates a system that can be readily modified to target modifications at different genomic sites and/or to target different modifications at the same site. Additionally, protocols have been established which enable simultaneous targeting of multiple genes. The majority of cells carrying the mutation present biallelic mutations in the targeted genes.

However, apparent flexibility in the base-pairing interactions between the sgRNA sequence and the genomic DNA target sequence allows imperfect matches to the target sequence to be cut by Cas9.

Modified versions of the Cas9 enzyme containing a single inactive catalytic domain, either RuvC- or HNH-, are called ‘nickases’. With only one active nuclease domain, the Cas9 nickase cuts only one strand of the target DNA, creating a single-strand break or ‘nick’. A single-strand break, or nick, is mostly repaired by single strand break repair mechanism involving proteins such as but not only, PARP (sensor) and XRCC1/LIG III complex (ligation). If a single strand break (SSB) is generated by topoisomerase I poisons or by drugs that trap PARP1 on naturally occurring SSBs then these could persist and when the cell enters into S-phase and the replication fork encounter such SSBs they will become single ended DSBs which can only be repaired by HR. However, two proximal, opposite strand nicks introduced by a Cas9 nickase are treated as a double-strand break, in what is often referred to as a ‘double nick’ CRISPR system. A double-nick, which is basically non-parallel DSB, can be repaired like other DSBs by HR or NHEJ depending on the desired effect on the gene target and the presence of a donor sequence and the cell cycle stage (HR is of much lower abundance and can only occur in S and G2 stages of the cell cycle). Thus, if specificity and reduced off-target effects are crucial, using the Cas9 nickase to create a double-nick by designing two sgRNAs with target sequences in close proximity and on opposite strands of the genomic DNA would decrease off-target effect as either sgRNA alone will result in nicks that are not likely to change the genomic DNA, even though these events are not impossible.

Modified versions of the Cas9 enzyme containing two inactive catalytic domains (dead Cas9, or dCas9) have no nuclease activity while still able to bind to DNA based on sgRNA specificity. The dCas9 can be utilized as a platform for DNA transcriptional regulators to activate or repress gene expression by fusing the inactive enzyme to known regulatory domains. For example, the binding of dCas9 alone to a target sequence in genomic DNA can interfere with gene transcription.

Additional variants of Cas9 which may be used by some embodiments of the invention include, but are not limited to, CasX and Cpf1. CasX enzymes comprise a distinct family of RNA-guided genome editors which are smaller in size compared to Cas9 and are found in bacteria (which is typically not found in humans), hence, are less likely to provoke the immune system/response in a human. Also, CasX utilizes a different PAM motif compared to Cas9 and therefore can be used to target sequences in which Cas9 PAM motifs are not found [see Liu J J et al., Nature. (2019) 566(7743):218-223]. Cpf1, also referred to as Cas12a, is especially advantageous for editing AT rich regions in which Cas9 PAMs (NGG) are much less abundant [see Li T et al., Biotechnol Adv. (2019) 37(1):21-27; Murugan K et al., Mol Cell. (2017) 68(1):15-25].

According to another embodiment, the CRISPR system may be fused with various effector domains, such as DNA cleavage domains. The DNA cleavage domain can be obtained from any endonuclease or exonuclease. Non-limiting examples of endonucleases from which a DNA cleavage domain can be derived include, but are not limited to, restriction endonucleases and homing endonucleases (see, for example, New England Biolabs Catalog or Belfort et al. (1997) Nucleic Acids Res.). In exemplary embodiments, the cleavage domain of the CRISPR system is a Fokl endonuclease domain or a modified Fokl endonuclease domain. In addition, the use of Homing Endonucleases (HE) is another alternative. HEs are small proteins (<300 amino acids) found in bacteria, archaea, and in unicellular eukaryotes. A distinguishing characteristic of HEs is that they recognize relatively long sequences (14-40 bp) compared to other site-specific endonucleases such as restriction enzymes (4-8 bp). HEs have been historically categorized by small conserved amino acid motifs. At least five such families have been identified: LAGLIDADG; GIY-YIG; HNH; His-Cys Box and PD-(D/E)xK, which are related to EDxHD enzymes and are considered by some as a separate family. At a structural level, the HNH and His-Cys Box share a common fold (designated ββα-metal) as do the PD-(D/E)xK and EDxHD enzymes. The catalytic and DNA recognition strategies for each of the families vary and lend themselves to different degrees to engineering for a variety of applications. See e.g. Methods Mol Biol. (2014) 1123:1-26. Exemplary Homing Endonucleases which may be used according to some embodiments of the invention include, without being limited to, I-CreI, I-TevI, I-HmuI, I-PpoI and I-Ssp68031.

Modified versions of CRISPR, e.g. dead CRISPR (dCRISPR-endonuclease), may also be utilized for CRISPR transcription inhibition (CRISPRi) or CRISPR transcription activation (CRISPRa) see e.g. Kampmann M., ACS Chem Biol. (2018) 13(2):406-416; La Russa M F and Qi L S., Mol Cell Biol. (2015) 35(22):3800-9].

Other versions of CRISPR which may be used according to some embodiments of the invention include genome editing using components from CRISPR systems together with other enzymes to directly install point mutations into cellular DNA or RNA.

Thus, according to one embodiment, the editing agent is DNA or RNA editing agent.

According to one embodiment, the DNA or RNA editing agent elicits base editing.

The term “base editing” as used herein refers to installing point mutations into cellular DNA or RNA without making double-stranded DNA breaks.

In base editing, DNA base editors typically comprise fusions between a catalytically impaired Cas nuclease and a base modification enzyme that operates on single-stranded DNA (ssDNA). Upon binding to its target DNA locus, base pairing between the gRNA and the target DNA strand leads to displacement of a small segment of single-stranded DNA in an ‘R loop’. DNA bases within this ssDNA bubble are modified by the base-editing enzyme (e.g. deaminase enzyme). To improve efficiency in eukaryotic cells, the catalytically disabled nuclease also generates a nick in the non-edited DNA strand, inducing cells to repair the non-edited strand using the edited strand as a template.

Two classes of DNA base editor have been described: cytosine base editors (CBEs) convert a C-G base pair into a T-A base pair, and adenine base editors (ABEs) convert an A-T base pair into a G-C base pair. Collectively, CBEs and ABEs can mediate all four possible transition mutations (C to T, A to G, T to C and G to A). Similarly in RNA, targeted adenosine conversion to inosine utilizes both antisense and Cas13-guided RNA-targeting methods.

According to one embodiment, the DNA or RNA editing agent comprises a catalytically inactive endonuclease (e.g. CRISPR-dCas).

According to one embodiment, the catalytically inactive endonuclease is an inactive Cas9 (e.g. dCas9).

According to one embodiment, the catalytically inactive endonuclease is an inactive Cas13 (e.g. dCas13).

According to one embodiment, the DNA or RNA editing agent comprises an enzyme which is capable of epigenetic editing (i.e. providing chemical changes to the DNA, the RNA or the histone proteins).

Exemplary enzymes include, but are not limited to, DNA methyltransferases, methylases, acetyltransferases. More specifically, exemplary enzymes include e.g. DNA (cytosine-5)-methyltransferase 3A (DNMT3a), Histone acetyltransferase p300, Ten-eleven translocation methylcytosine dioxygenase 1 (TET1), Lysine (K)-specific demethylase 1A (LSD1) and Calcium and integrin binding protein 1 (CIB1).

In addition to the catalytically disabled nuclease, the DNA or RNA editing agents of the invention may also comprise a nucleobase deaminase enzyme and/or a DNA glycosylase inhibitor.

According to a specific embodiment, the DNA or RNA editing agents comprise BE1 (APOBEC1-XTEN-dCas9), BE2 (APOBEC1-XTEN-dCas9-UGI) or BE3 (APOBEC-XTEN-dCas9(A840H)-UGI), along with sgRNA. APOBEC1 is a deaminase full length or catalytically active fragment, XTEN is a protein linker, UGI is uracil DNA glycosylase inhibitor to prevent the subsequent U:G mismatch from being repaired back to a C:G base pair and dCas9 (A840H) is a nickase in which the dCas9 was reverted to restore the catalytic activity of the HNH domain which nicks only the non-edited strand, simulating newly synthesized DNA and leading to the desired U:A product.

Additional enzymes which can be used for base editing according to some embodiments of the invention are specified in Rees and Liu, Nature Reviews Genetics (2018) 19:770-788, incorporated herein by reference in its entirety.

There are a number of publically available tools to help choose and/or design target sequences as well as lists of bioinformatically determined unique sgRNAs for different genes in different species such as, but not limited to, the Feng Zhang lab's Target Finder, the Michael Boutros lab's Target Finder (E-CRISP), the RGEN Tools: Cas-OFFinder, the CasFinder: Flexible algorithm for identifying specific Cas9 targets in genomes and the CRISPR Optimal Target Finder.

To use the CRISPR system, both sgRNA and a Cas endonuclease (e.g. Cas9) should be expressed or present (e.g., as a ribonucleoprotein complex) in a target cell. The insertion vector can contain both cassettes on a single plasmid or the cassettes are expressed from two separate plasmids. CRISPR plasmids are commercially available such as the px330 plasmid from Addgene (Cambridge, Mass.). Use of clustered regularly interspaced short palindromic repeats (CRISPR)-associated (Cas)-guide RNA technology and a Cas endonuclease for modifying plant genomes are also at least disclosed by Svitashev et al., 2015, Plant Physiology, 169 (2): 931-945; Kumar and Jain, 2015, J Exp Bot 66: 47-57; and in U.S. Patent Application Publication No. 20150082478, which is specifically incorporated herein by reference in its entirety. Cas endonucleases that can be used to effect DNA editing with sgRNA include, but are not limited to, Cas9, Cpf1 (Zetsche et al., 2015, Cell. 163(3):759-71), C2c1, C2c2, C2c3, cms1 (Shmakov et al., Mol Cell. 2015 Nov. 5; 60(3):385-97) and Cas 13A/B (Barrangoul et al., 2017, Molecular cell, 65: 582-584; Abudayyeh et al., 2017, Nature 550: 280-284). The Cas 13 A OR B (Cas 13A/B) can recognize and cleave RNA, not DNA. this could be applied when RNA-degradation (RNAI-like) is desired.

“Hit and run” or “in-out”—involves a two-step recombination procedure. In the first step, an insertion-type vector containing a dual positive/negative selectable marker cassette is used to introduce the desired sequence alteration. The insertion vector contains a single continuous region of homology to the targeted locus and is modified to carry the mutation of interest. This targeting construct is linearized with a restriction enzyme at a one site within the region of homology, introduced into the cells, and positive selection is performed to isolate homologous recombination events. The DNA carrying the homologous sequence can be provided as a plasmid, single or double stranded oligo. These homologous recombinants contain a local duplication that is separated by intervening vector sequence, including the selection cassette. In the second step, targeted clones are subjected to negative selection to identify cells that have lost the selection cassette via intrachromosomal recombination between the duplicated sequences. The local recombination event removes the duplication and, depending on the site of recombination, the allele either retains the introduced mutation or reverts to wild type. The end result is the introduction of the desired modification without the retention of any exogenous sequences.

The “double-replacement” or “tag and exchange” strategy—involves a two-step selection procedure similar to the hit and run approach, but requires the use of two different targeting constructs. In the first step, a standard targeting vector with 3′ and 5′ homology arms is used to insert a dual positive/negative selectable cassette near the location where the mutation is to be introduced. After the system component have been introduced to the cell and positive selection applied, HR events could be identified. Next, a second targeting vector that contains a region of homology with the desired mutation is introduced into targeted clones, and negative selection is applied to remove the selection cassette and introduce the mutation. The final allele contains the desired mutation while eliminating unwanted exogenous sequences.

Site-Specific Recombinases—The Cre recombinase derived from the P1 bacteriophage and Flp recombinase derived from the yeast Saccharomyces cerevisiae are site-specific DNA recombinases each recognizing a unique 34 base pair DNA sequence (termed “Lox” and “FRT”, respectively) and sequences that are flanked with either Lox sites or FRT sites can be readily removed via site-specific recombination upon expression of Cre or Flp recombinase, respectively. For example, the Lox sequence is composed of an asymmetric eight base pair spacer region flanked by 13 base pair inverted repeats. Cre recombines the 34 base pair lox DNA sequence by binding to the 13 base pair inverted repeats and catalyzing strand cleavage and re-ligation within the spacer region. The staggered DNA cuts made by Cre in the spacer region are separated by 6 base pairs to give an overlap region that acts as a homology sensor to ensure that only recombination sites having the same overlap region recombine.

Basically, the site specific recombinase system offers means for the removal of selection cassettes after homologous recombination events. This system also allows for the generation of conditional altered alleles that can be inactivated or activated in a temporal or tissue-specific manner. Of note, the Cre and Flp recombinases leave behind a Lox or FRT “scar” of 34 base pairs. The Lox or FRT sites that remain are typically left behind in an intron or 3′ UTR of the modified locus, and current evidence suggests that these sites usually do not interfere significantly with gene function.

Thus, Cre/Lox and Flp/FRT recombination involves introduction of a targeting vector with 3′ and 5′ homology arms containing the mutation of interest, two Lox or FRT sequences and typically a selectable cassette placed between the two Lox or FRT sequences. Positive selection is applied and homologous recombination events that contain targeted mutation are identified. Transient expression of Cre or Flp in conjunction with negative selection results in the excision of the selection cassette and selects for cells where the cassette has been lost. The final targeted allele contains the Lox or FRT scar of exogenous sequences.

According to a specific embodiment, the DNA editing agent is a non-integrated DNA editing agent.

According to a specific embodiment, the DNA editing agent comprises a DNA targeting module (e.g., sgRNA).

According to a specific embodiment, the DNA editing agent does not comprise an endonuclease.

According to a specific embodiment, the DNA editing agent comprises an endonuclease.

According to a specific embodiment, the DNA editing agent comprises a catalytically inactive endonuclease.

According to a specific embodiment, the DNA editing agent comprises a nuclease (e.g. an endonuclease) and a DNA targeting module (e.g., sgRNA).

According to a specific embodiment, the DNA editing agent is CRISPR/endonuclease.

According to a specific embodiment, the DNA editing agent comprises at least one sgRNA (e.g. one, two, three, four or more sgRNAs).

According to a specific embodiment, the DNA editing agent comprises two sgRNAs.

According to a specific embodiment, the DNA editing agent comprises two pairs of sgRNAs.

According to a specific embodiment, the DNA editing agent is CRISPR/Cas, e.g. sgRNA and Cas9 or a sgRNA and dCas9.

Exemplary sgRNA sequences that may be found within expression constructs, e.g. plasmids, include but are not limited to, the ones provided below:

sgRNA #6
(SEQ ID NO: 51)
AAAACCGAATTGAAATCATT
sgRNA #7
(SEQ ID NO: 52)
TGCCTAATAGGGGCAATGCC
sgRNA #11
(SEQ ID NO: 53)
TTCAAGGACAGGTTTCACCT
sgRNA #12
(SEQ ID NO: 54)
CAACAAGTGCATTAAAGTTG
sgRNA #13
(SEQ ID NO: 55)
AAAGAAAATGGACGCAAAAT
sgRNA #14
(SEQ ID NO: 56)
AAAAAATGCATGGACTCCTC
sgRNA #37
(SEQ ID NO: 57)
CGTATGCATTGTTCAAGGAA
sgRNA #38
(SEQ ID NO: 58)
AAAGAAAATGGACGCAAGAT
sgRNA-1
(SEQ ID NO: 59)
TTTGCACAATTAATCATTAAGGG
sgRNA-2
(SEQ ID NO: 60)
CAAGAAGTCCTGCGGATGAATGG
sgRNA-3
(SEQ ID NO: 61)
ACTTGTACATAAATCAAATTGGG
sgRNA-4
(SEQ ID NO: 62)
CAAATTGGGACTGCCAAAGAAGG
sgRNA-5
(SEQ ID NO: 63)
GAAGTCCTGCATATGAATGAAGG
sgRNA-6
(SEQ ID NO: 64)
GACGGGCGGACGACATCCTTTGG
sgRNA-7
(SEQ ID NO: 65)
TTGGTGATTGAATTGGGGATTGG
sgRNA-8
(SEQ ID NO: 66)
GGGAGTATTTACTCTTCCAAAGG
sgRNA-9
(SEQ ID NO: 67)
TCAACAAGTGCTTTAAAGTTGGG
sgRNA-10
(SEQ ID NO: 68)
TGCTTTAAAGTTGGGGATTTGGG
sgRNA-11
(SEQ ID NO: 69)
AAAATAGGATCGTGCCTGATAGG
sgRNA-12
(SEQ ID NO: 70)
CGAACTGTTGAAAATGTGTTTGG
sgRNA-13
(SEQ ID NO: 71)
CCTCGGGGAAGAGTCTGCCGTGG
sgRNA-14
(SEQ ID NO: 72)
ACTTTGTACAGTGTCCCGAACGG
sgRNA-15
(SEQ ID NO: 73)
ATTAGAACGTCCCACCATTCAGG
sgRNA-16
(SEQ ID NO: 74)
ATGCGACGGCCCGAATACCATGG
sgRNA-17
(SEQ ID NO: 75)
CATTCGGAAGAGTTGCTTTCAGG
sgRNA-18
(SEQ ID NO: 76)
GTCTATGGTATTCAGGCCATCGG
sgRNA-19
(SEQ ID NO: 77)
AGCGGATTGGTGACTGAACTGGG
sgRNA-20
(SEQ ID NO: 78)
TCGGAAGAGTTGCTTTCAGGTGG

According to a specific embodiment, the DNA or RNA editing agent elicits base editing.

According to a specific embodiment, the DNA or RNA editing agent comprises an enzyme for epigenetic editing.

According to a specific embodiment, the DNA editing agent is TALEN.

According to a specific embodiment, the DNA editing agent is ZFN.

According to a specific embodiment, the DNA editing agent is meganuclease.

According to a specific embodiment, the DNA editing agent modifies a single methyltransferase target sequence (e.g. XMT, MXMT or DXMT).

According to a specific embodiment, the DNA editing agent modifies two, three, four, five or more methyltransferase target sequences (e.g. XMT, MXMT or DXMT).

According to a specific embodiment, a single DNA editing agent targets a number of genes (e.g., 2-10 genes, e.g., 5-10 genes, e.g. 2-5 genes, e.g., 4-5 genes, e.g. 3-5 genes, e.g., 5 genes).

According to a specific embodiment, the DNA editing agent is directed to a nucleic sequence that is at least 50-99% identical, e.g. 51-99%, 53-99%, 55-99%, 57-99%, 59-99%, 61-99%, 63-99%, 65-99%, 67-99%, 69-99%, 71-99%, 73-99%, 75-99%, 77-99%, 79-99%, 81-99%, 83-99%, 85-99%, 87-99%, 89-99%, 91-99%, 93-99%, 95-99%, 97-99%, 98-99% identical, e.g. 50%, 52%, 54%, 56%, 58%, 60%, 62%, 64%, 66%, 68%, 70%, 72%, 74%, 76%, 78%, 80%, 82%, 84%, 86%, 88%, 90%, 91%, 92%, 93%, 94%, 95%, 96%, 97%, 98%, 99% or 100% identical, e.g. 99% identical) between Cc09_g06970 (set forth in SEQ ID NO: 9), Cc09_g06960 (set forth in SEQ ID NO: 7), Cc00_g24720 (set forth in SEQ ID NO: 1), Cc09_g06950 (set forth in SEQ ID NO: 5), Cc01_g00720 (set forth in SEQ ID NO: 3) and Cc02_g09350 (set forth in SEQ ID NO: 11), over a length of 5-100 nucleotides (e.g. 5-50 nucleotides, e.g. 5-25 nucleotides, e.g. 10-25 nucleotides) as determined by local alignment (e.g. CLUSTAL multiple sequence alignment by MUSCLE).

As used herein, “sequence identity” or “identity” in the context of two nucleic acid or polypeptide sequences includes reference to the residues in the two sequences which are the same when aligned. When percentage of sequence identity is used in reference to proteins it is recognized that residue positions which are not identical often differ by conservative amino acid substitutions, where amino acid residues are substituted for other amino acid residues with similar chemical properties (e.g. charge or hydrophobicity) and therefore do not change the functional properties of the molecule. Where sequences differ in conservative substitutions, the percent sequence identity may be adjusted upwards to correct for the conservative nature of the substitution. Sequences which differ by such conservative substitutions are considered to have “sequence similarity” or “similarity”. Means for making this adjustment are well-known to those of skill in the art. Typically this involves scoring a conservative substitution as a partial rather than a full mismatch, thereby increasing the percentage sequence identity. Thus, for example, where an identical amino acid is given a score of 1 and a non-conservative substitution is given a score of zero, a conservative substitution is given a score between zero and 1. The scoring of conservative substitutions is calculated, e.g., according to the algorithm of Henikoff S and Henikoff J G. [Amino acid substitution matrices from protein blocks. Proc. Natl. Acad. Sci. U.S.A. 1992, 89(22): 10915-9].

Identity (e.g., percent homology) can be determined using any homology comparison software, including for example, the BlastN software of the National Center of Biotechnology Information (NCBI) such as by using default parameters.

According to some embodiments of the invention, the identity is a global identity, i.e., an identity over the entire amino acid or nucleic acid sequences of the invention and not over portions thereof.

According to some embodiments of the invention, the term “homology” or “homologous” refers to identity of two or more nucleic acid sequences; or identity of two or more amino acid sequences; or the identity of an amino acid sequence to one or more nucleic acid sequence.

According to some embodiments of the invention, the homology is a global homology, i.e., a homology over the entire amino acid or nucleic acid sequences of the invention and not over portions thereof.

The degree of homology or identity between two or more sequences can be determined using various known sequence comparison tools. For example, when starting with a polynucleotide sequence and comparing to other polynucleotide sequences the EMBOSS-6.0.1 Needleman-Wunsch algorithm (available from embos s(dot)sourceforge(dot)net/apps/cvs/emboss/apps/needle(dot)html) can be used with the following default parameters: (EMBOSS-6.0.1) gapopen=10; gapextend=0.5; datafile=EDNAFULL; brief=YES.

According to a specific embodiment, the DNA editing agent is directed to a nucleic acid segment comprised in a nucleic acid sequence as set forth in any one of SEQ ID NOs: 25-50.

According to a specific embodiment, the DNA editing agent is directed to a nucleic acid segment comprised in a nucleic acid sequence as set forth in any one of SEQ ID NOs: 26-31, 33-36, 38-41, 43-45, 47-48 or 50.

According to a specific embodiment, the DNA editing agent is directed to a partial sequence of the nucleic acid sequence as set forth in any one of SEQ ID NOs: 26-31, 33-36, 38-41, 43-45, 47-48 or 50.

According to a specific embodiment, the DNA editing agent is directed to the entire nucleic acid sequence as set forth in any one of SEQ ID NOs: 26-31, 33-36, 38-41, 43-45, 47-48 or 50.

According to a specific embodiment, the DNA editing agent modifies the target sequence methyltransferase (e.g. XMT, MXMT and/or DXMT) and is devoid of “off target” activity, i.e., does not modify other sequences in the coffee genome.

According to a specific embodiment, the DNA editing agent comprises an “off target activity” on a non-essential gene in the coffee genome.

Non-essential refers to a gene that when modified with the DNA editing agent does not affect the phenotype of the target genome in an agriculturally valuable manner (e.g., flavor, biomass, yield, biotic/abiotic stress, pest resistance, tolerance and the like).

According to one embodiment, the DNA editing agent is linked to a reporter for monitoring expression in a plant cell.

According to one embodiment, the reporter is a fluorescent reporter protein.

The term “a fluorescent protein” refers to a polypeptide that emits fluorescence and is typically detectable by flow cytometry, microscopy or any fluorescent imaging system, therefore can be used as a basis for selection of cells expressing such a protein.

Examples of fluorescent proteins that can be used as reporters are, without being limited to, the Green Fluorescent Protein (GFP), the Blue Fluorescent Protein (BFP) and the red fluorescent proteins (e.g. dsRed, mCherry, RFP). A non-limiting list of fluorescent or other reporters includes proteins detectable by luminescence (e.g. luciferase) or colorimetric assay (e.g. GUS). According to a specific embodiment, the fluorescent reporter is a red fluorescent protein (e.g. dsRed, mCherry, RFP) or GFP.

A review of new classes of fluorescent proteins and applications can be found in Trends in Biochemical Sciences [Rodriguez, Erik A.; Campbell, Robert E.; Lin, John Y.; Lin, Michael Z; Miyawaki, Atsushi; Palmer, Amy E.; Shu, Xiaokun; Zhang, Jin; Tsien, Roger E “The Growing and Glowing Toolbox of Fluorescent and Photoactive Proteins”. Trends in Biochemical Sciences. doi:10. 1016/j.tibs.2016.09.010].

Any method known in the art for linking (e.g. a DNA editing agent to a reporter) may be used according to the present teachings.

The term “linked” as used herein refers to the joining of nucleic acid sequences such that one sequence can provide a required function to a linked sequence. In the context of a reporter, linked means that the reporter is connected to a sequence of a DNA editing agent such that the transcription of the reporter is controlled and regulated by transcription of the DNA editing agent. Additionally or alternatively, linked may also mean that the reporter and the sequence of a DNA editing agent are transcribed from the same plasmid or from multiple plasmids (co-transfection), e.g. using two different promoters. Accordingly, linkage may be a transcriptional fusion, a translational fusion or may be non-fused.

The DNA editing agent is typically introduced into the plant cell using expression vectors.

Thus, according to an aspect of the invention there is provided a nucleic acid construct comprising a nucleic acid sequence encoding a DNA editing agent directed towards at least one component of a caffeine biosynthesis pathway being operably linked to a cis-acting regulatory element (e.g. plant promoter) for expressing the DNA editing agent in a cell of a coffee plant.

It will be appreciated that the present teachings also relate to introducing the DNA editing agent using DNA-free methods such as mRNA+sgRNA transfection or RNP transfection.

According to a specific embodiment, the nucleic acid construct comprises a nucleic acid sequence encoding an endonuclease of a DNA editing agent (e.g., Cas9 or the endonucleases described above).

Constructs useful in the methods according to some embodiments may be constructed using recombinant DNA technology well known to persons skilled in the art. Such constructs may be commercially available, suitable for transforming into plants and suitable for expression of the gene of interest in the transformed cells.

According to another specific embodiment, the endonuclease and the sgRNA are encoded from different constructs whereby each is operably linked to a cis-acting regulatory element active in plant cells (e.g., promoter).

In a particular embodiment of some embodiments of the invention the regulatory element is a plant-expressible promoter.

As used herein the phrase “plant-expressible” refers to a promoter sequence, including any additional regulatory elements added thereto or contained therein, is at least capable of inducing, conferring, activating or enhancing expression in a plant cell, tissue or organ, preferably a monocotyledonous or dicotyledonous plant cell, tissue, or organ. Examples of promoters useful for the methods of some embodiments of the invention include, but are not limited to, Actin, CANV 35S, CaMV19S, GOS2. Promoters which are active in various tissues, or developmental stages can also be used.

According to a specific embodiment, promoters in the nucleic acid construct comprise a Pol3 promoter. Examples of Pol3 promoters include, but are not limited to, AtU6-29, AtU626, AtU3B, AtU3d, TaU6.

According to a specific embodiment, promoters in the nucleic acid construct comprise a Pol2 promoter. Examples of Pol2 promoters include, but are not limited to, CaMV 35S, CaMV 19S, ubiquitin, CVMV.

According to a specific embodiment, promoters in the nucleic acid construct comprise a 35S promoter.

According to a specific embodiment, promoters in the nucleic acid construct comprise a U6 promoter.

According to a specific embodiment, promoters in the nucleic acid construct comprise a Pol 3 (e.g., U6) promoter operatively linked to the nucleic acid agent encoding at least one sgRNA and/or a Pol2 (e.g., CaMV35S) promoter operatively linked to the nucleic acid sequence encoding the genome editing agent or the nucleic acid sequence encoding the fluorescent reporter (as described in a specific embodiment below).

According to a specific embodiment, the promoter is a U6 pol 3 promoter.

Nucleic acid sequences of the polypeptides of some embodiments of the invention may be optimized for plant expression. Examples of such sequence modifications include, but are not limited to, an altered G/C content to more closely approach that typically found in the plant species of interest, and the removal of codons atypically found in the plant species commonly referred to as codon optimization.

Plant cells may be transformed stably or transiently with the nucleic acid constructs of some embodiments of the invention. In stable transformation, the nucleic acid molecule of some embodiments of the invention is integrated into the plant genome and as such it represents a stable and inherited trait. In transient transformation, the nucleic acid molecule is expressed by the cell transformed but it is not integrated into the genome and as such it represents a transient CRISPR-Cas9 system.

According to a specific embodiment, the plant is transiently transfected with a DNA editing agent.

According to a specific embodiment, the construct is useful for transient expression (Helens et al., 2005, Plant Methods 1:13). Methods of transient transformation are further described hereinbelow.

Various cloning kits can be used according to the teachings of some embodiments of the invention [e.g., GoldenGate assembly kit by New England Biolabs (NEB)].

According to a specific embodiment the nucleic acid construct is a binary vector. Examples for binary vectors are pBIN19, pBI101, pBinAR, pGPTV, pCAMBIA, pBIB-HYG, pBecks, pGreen or pPZP (Hajukiewicz, P. et al., Plant Mol. Biol. 25, 989 (1994), and Hellens et al, Trends in Plant Science 5, 446 (2000)).

Examples of other vectors to be used in other methods of DNA delivery (e.g. transfection, electroporation, bombardment, viral inoculation) are: pGE-sgRNA (Zhang et al. Nat. Comms. 2016 7:12697), pJIT163-Ubi-Cas9 (Wang et al. Nat. Biotechnol 2004 32, 947-951), pICH47742::2x35S-5′UTR-hCas9(STOP)-NOST (Belhan et al. Plant Methods 2013 11; 9(1):39).

There are several methods of introducing DNA into plant cells e.g., using protoplasts, and the skilled artisan will know which to select.

The delivery of nucleic acids may be introduced into a plant cell in embodiments of the invention by any method known to those of skill in the art, including, for example and without limitation: by transformation of protoplasts (See, e.g., U.S. Pat. No. 5,508,184); by desiccation/inhibition-mediated DNA uptake (See, e.g., Potrykus et al. (1985) Mol. Gen. Genet. 199:183-8); by electroporation (See, e.g., U.S. Pat. No. 5,384,253); by agitation with silicon carbide fibers (See, e.g., U.S. Pat. Nos. 5,302,523 and 5,464,765); by Agrobacterium-mediated transformation (See, e.g., U.S. Pat. Nos. 5,563,055, 5,591,616, 5,693,512, 5,824,877, 5,981,840, and 6,384,301); by acceleration of DNA-coated particles (See, e.g., U.S. Pat. Nos. 5,015,580, 5,550,318, 5,538,880, 6,160,208, 6,399,861, and 6,403,865) and by Nanoparticles, nanocarriers and cell penetrating peptides (WO201126644A2; WO2009046384A1; WO2008148223A1) in the methods to deliver DNA, RNA, Peptides and/or proteins or combinations of nucleic acids and peptides into plant cells.

Other methods of transfection include the use of transfection reagents (e.g. Lipofectin, ThermoFisher), dendrimers (Kukowska-Latallo, J. F. et al., 1996, Proc. Natl. Acad. Sci. USA93, 4897-902), cell penetrating peptides (Mae et al., 2005, Internalisation of cell-penetrating peptides into tobacco protoplasts, Biochimica et Biophysica Acta 1669(2):101-7) or polyamines (Zhang and Vinogradov, 2010, Short biodegradable polyamines for gene delivery and transfection of brain capillary endothelial cells, J Control Release, 143(3):359-366).

According to a specific embodiment, the introduction of DNA into plant cells (e.g., protoplasts) is effected by electroporation.

According to a specific embodiment, the introduction of DNA into plant cells (e.g., embryogenic cells) is effected by bombardment/biolistics.

According to a specific embodiment, for introducing DNA into protoplasts the method comprises polyethylene glycol (PEG)-mediated DNA uptake. For further details see Karesch et al. (1991) Plant Cell Rep. 9:575-578; Mathur et al. (1995) Plant Cell Rep. 14:221-226; Negrutiu et al. (1987) Plant Cell Mol. Biol. 8:363-373. Protoplasts are then cultured under conditions that allowed them to grow cell walls, start dividing to form a callus, develop shoots and roots, and regenerate whole plants.

Transient transformation can also be effected by viral infection using modified plant viruses.

Viruses that have been shown to be useful for the transformation of plant hosts include CaMV, TMV, TRV and BV. Transformation of plants using plant viruses is described in U.S. Pat. No. 4,855,237 (BGV), EP-A 67,553 (TMV), Japanese Published Application No. 63-14693 (TMV), EPA 194,809 (BV), EPA 278,667 (BV); and Gluzman, Y. et al., Communications in Molecular Biology: Viral Vectors, Cold Spring Harbor Laboratory, New York, pp. 172-189 (1988). Pseudovirus particles for use in expressing foreign DNA in many hosts, including plants, is described in WO 87/06261.

Construction of plant RNA viruses for the introduction and expression of non-viral exogenous nucleic acid sequences in plants is demonstrated by the above references as well as by Dawson, W. O. et al., Virology (1989) 172:285-292; Takamatsu et al. EMBO J. (1987) 6:307-311; French et al. Science (1986) 231:1294-1297; and Takamatsu et al. FEBS Letters (1990) 269:73-76.

When the virus is a DNA virus, suitable modifications can be made to the virus itself. Alternatively, the virus DNA can first be cloned into a bacterial plasmid for ease of constructing the desired viral vector with the foreign DNA. The virus DNA can then be excised from the plasmid. If the virus is a DNA virus, a bacterial origin of replication can be attached to the viral DNA, which is then replicated by the bacteria. Transcription and translation of this DNA will produce the coat protein which will encapsidate the viral DNA. If the virus is an RNA virus, the virus is generally cloned as a cDNA and inserted into a plasmid. The plasmid is then used to make all of the constructions. The RNA virus is then produced by transcribing the viral sequence of the plasmid and translation of the viral genes to produce the coat protein(s) which encapsidate the viral RNA.

Construction of plant RNA viruses for the introduction and expression in plants of non-viral exogenous nucleic acid sequences such as those included in the construct of some embodiments of the invention is demonstrated by the above references as well as in U.S. Pat. No. 5,316,931.

In one embodiment, a plant viral nucleic acid is provided in which the native coat protein coding sequence has been deleted from a viral nucleic acid, a non-native plant viral coat protein coding sequence and a non-native promoter, preferably the subgenomic promoter of the non-native coat protein coding sequence, capable of expression in the plant host, packaging of the recombinant plant viral nucleic acid, and ensuring a systemic infection of the host by the recombinant plant viral nucleic acid, has been inserted. Alternatively, the coat protein gene may be inactivated by insertion of the non-native nucleic acid sequence within it, such that a protein is produced. The recombinant plant viral nucleic acid may contain one or more additional non-native subgenomic promoters. Each non-native subgenomic promoter is capable of transcribing or expressing adjacent genes or nucleic acid sequences in the plant host and incapable of recombination with each other and with native subgenomic promoters. Non-native (foreign) nucleic acid sequences may be inserted adjacent the native plant viral subgenomic promoter or the native and a non-native plant viral subgenomic promoters if more than one nucleic acid sequence is included. The non-native nucleic acid sequences are transcribed or expressed in the host plant under control of the subgenomic promoter to produce the desired products.

In a second embodiment, a recombinant plant viral nucleic acid is provided as in the first embodiment except that the native coat protein coding sequence is placed adjacent one of the non-native coat protein subgenomic promoters instead of a non-native coat protein coding sequence.

In a third embodiment, a recombinant plant viral nucleic acid is provided in which the native coat protein gene is adjacent its subgenomic promoter and one or more non-native subgenomic promoters have been inserted into the viral nucleic acid. The inserted non-native subgenomic promoters are capable of transcribing or expressing adjacent genes in a plant host and are incapable of recombination with each other and with native subgenomic promoters. Non-native nucleic acid sequences may be inserted adjacent the non-native subgenomic plant viral promoters such that the sequences are transcribed or expressed in the host plant under control of the subgenomic promoters to produce the desired product.

In a fourth embodiment, a recombinant plant viral nucleic acid is provided as in the third embodiment except that the native coat protein coding sequence is replaced by a non-native coat protein coding sequence.

The viral vectors are encapsidated by the coat proteins encoded by the recombinant plant viral nucleic acid to produce a recombinant plant virus. The recombinant plant viral nucleic acid or recombinant plant virus is used to infect appropriate host plants. The recombinant plant viral nucleic acid is capable of replication in the host, systemic spread in the host, and transcription or expression of foreign gene(s) (isolated nucleic acid) in the host to produce the desired protein.

Regardless of the transformation/infection method employed, the present teachings further relate to any cell e.g., a plant cell (e.g., protoplast) comprising the nucleic acid construct(s) as described herein.

Following transformation, cells are subjected to selection methods. Any method known in the art may be used to select transformed cells.

Following selection, positively selected pools of transformed plant cells, (e.g., protoplasts) are collected and an aliquot can be used for testing the DNA editing event.

Alternatively (or following optional validating) the clones are cultivated in the absence of selection (e.g., antibiotics for a selection marker) until they develop into colonies i.e., clones (at least 28 days) and micro-calli. Following at least 60-100 days in culture (e.g., at least 70 days, at least 80 days), a portion of the cells of the calli are analyzed (validated) for: the DNA editing event and the presence of the DNA editing agent, namely, loss of DNA sequences encoding for the DNA editing agent, pointing to the transient nature of the method.

Thus, clones are validated for the presence of a DNA editing event also referred to herein as “mutation” or “edit”, dependent on the type of editing sought e.g., insertion, deletion, insertion-deletion (Indel), inversion, substitution and combinations thereof.

According to a specific embodiment, the mutation comprises a modification of about 1-500 nucleotides, about 1-250 nucleotides, about 1-150 nucleotides, about 1-100 nucleotides, about 1-50 nucleotides, about 1-25 nucleotides, about 1-10 nucleotides, about 10-250 nucleotides, about 10-200 nucleotides, about 10-150 nucleotides, about 10-100 nucleotides, about 10-50 nucleotides, about 1-50 nucleotides, about 1-10 nucleotides, about 50-150 nucleotides, about 50-100 nucleotides or about 100-200 nucleotides (as compared to the nucleotide sequence of the wild type component of the caffeine biosynthesis pathway, e.g. XMT/DXMT/MXMT).

According to one embodiment, the mutation comprises a modification of at most 1, 2, 3, 4, 5, 6, 7, 8, 9, 10, 11, 12, 13, 14, 15, 16, 17, 18, 19, 20, 22, 24, 26, 28, 30, 32, 34, 36, 38, 40, 42, 44, 46, 48, 50, 60, 70, 80, 90, 100, 110, 120, 130, 140, 150, 160, 170, 180, 190, 200, 250, 300, 350, 400, 450 or at most 500 nucleotides (as compared to the nucleotide sequence of the wild type component of the caffeine biosynthesis pathway, e.g. XMT/DXMT/MXMT).

According to one embodiment, the modification can be in a consecutive nucleic acid sequence (e.g. at least 5, 10, 20, 30, 40, 50, 100, 150, 200, 300, 400, 500 bases).

According to one embodiment, the modification can be in a non-consecutive manner, e.g. throughout a 10, 20, 50, 100, 150, 200, 500, 1000, 2000, 5000 nucleic acid sequence.

According to a specific embodiment, the mutation comprises a modification of at most 200 nucleotides.

According to a specific embodiment, the mutation comprises a modification of at most 150 nucleotides.

According to a specific embodiment, the mutation comprises a modification of at most 100 nucleotides.

According to a specific embodiment, the mutation comprises a modification of at most 50 nucleotides.

According to a specific embodiment, the mutation comprises a modification of at most 25 nucleotides.

According to a specific embodiment, the mutation comprises a modification of at most 20 nucleotides.

According to a specific embodiment, the mutation comprises a modification of at most 15 nucleotides.

According to a specific embodiment, the mutation comprises a modification of at most 10 nucleotides.

According to a specific embodiment, the mutation comprises a modification of at most 5 nucleotides.

According to a specific embodiment, the mutation comprises a modification of at most 2 nucleotides.

According to a specific embodiment, the mutation comprises a modification of one nucleotide.

According to one embodiment, the mutation is such that the recognition/cut site/PAM motif of the target molecule is modified to abolish the original PAM recognition site.

According to a specific embodiment, the mutation is in at least 1, 2, 3, 4, 5, 6, 7, 8, 9, 10 or more nucleic acids in a PAM motif.

According to one embodiment, the mutation comprises an insertion.

According to a specific embodiment, the insertion comprises an insertion of about 1-500 nucleotides, about 1-250 nucleotides, about 1-150 nucleotides, about 1-100 nucleotides, about 1-50 nucleotides, about 1-25 nucleotides, about 1-10 nucleotides, about 10-250 nucleotides, about 10-200 nucleotides, about 10-150 nucleotides, about 10-100 nucleotides, about 10-50 nucleotides, about 1-50 nucleotides, about 1-10 nucleotides, about 50-150 nucleotides, about 50-100 nucleotides or about 100-200 nucleotides (as compared to the nucleotide sequence of the wild type component of the caffeine biosynthesis pathway, e.g. XMT/DXMT/MXMT).

According to one embodiment, the insertion comprises an insertion of at most 1, 2, 3, 4, 5, 6, 7, 8, 9, 10, 11, 12, 13, 14, 15, 16, 17, 18, 19, 20, 22, 24, 26, 28, 30, 32, 34, 36, 38, 40, 42, 44, 46, 48, 50, 60, 70, 80, 90, 100, 110, 120, 130, 140, 150, 160, 170, 180, 190, 200, 250, 300, 350, 400 or at most 500 nucleotides (as compared to the nucleotide sequence of the wild type component of the caffeine biosynthesis pathway, e.g. XMT/DXMT/MXMT).

According to a specific embodiment, the insertion comprises an insertion of at most 200 nucleotides.

According to a specific embodiment, the insertion comprises an insertion of at most 150 nucleotides.

According to a specific embodiment, the insertion comprises an insertion of at most 100 nucleotides.

According to a specific embodiment, the insertion comprises an insertion of at most 50 nucleotides.

According to a specific embodiment, the insertion comprises an insertion of at most 25 nucleotides.

According to a specific embodiment, the insertion comprises an insertion of at most 20 nucleotides.

According to a specific embodiment, the insertion comprises an insertion of at most 15 nucleotides.

According to a specific embodiment, the insertion comprises an insertion of at most 10 nucleotides.

According to a specific embodiment, the insertion comprises an insertion of at most 5 nucleotides.

According to a specific embodiment, the insertion comprises an insertion of at most 2 nucleotides.

According to a specific embodiment, the insertion comprises an insertion of one nucleotide.

According to one embodiment, the mutation comprises a deletion.

According to a specific embodiment, the deletion comprises a deletion of about 1-500 nucleotides, about 1-250 nucleotides, about 1-150 nucleotides, about 1-100 nucleotides, about 1-50 nucleotides, about 1-25 nucleotides, about 1-10 nucleotides, about 10-250 nucleotides, about 10-200 nucleotides, about 10-150 nucleotides, about 10-100 nucleotides, about 10-50 nucleotides, about 1-50 nucleotides, about 1-10 nucleotides, about 50-150 nucleotides, about 50-100 nucleotides or about 100-200 nucleotides (as compared to the nucleotide sequence of the wild type component of the caffeine biosynthesis pathway, e.g. XMT/DXMT/MXMT).

According to one embodiment, the deletion comprises a deletion of at most 1, 2, 3, 4, 5, 6, 7, 8, 9, 10, 11, 12, 13, 14, 15, 16, 17, 18, 19, 20, 22, 24, 26, 28, 30, 32, 34, 36, 38, 40, 42, 44, 46, 48, 50, 60, 70, 80, 90, 100, 110, 120, 130, 140, 150, 160, 170, 180, 190, 200, 250, 300, 350, 400, 450 or at most 500 nucleotides (as compared to the nucleotide sequence of the wild type component of the caffeine biosynthesis pathway, e.g. XMT/DXMT/MXMT).

According to a specific embodiment, the deletion comprises a deletion of at most 200 nucleotides.

According to a specific embodiment, the deletion comprises a deletion of at most 150 nucleotides.

According to a specific embodiment, the deletion comprises a deletion of at most 100 nucleotides.

According to a specific embodiment, the deletion comprises a deletion of at most 50 nucleotides.

According to a specific embodiment, the deletion comprises a deletion of at most 25 nucleotides.

According to a specific embodiment, the deletion comprises a deletion of at most 20 nucleotides.

According to a specific embodiment, the deletion comprises a deletion of at most 15 nucleotides.

According to a specific embodiment, the deletion comprises a deletion of at most 10 nucleotides.

According to a specific embodiment, the deletion comprises a deletion of at most 5 nucleotides.

According to a specific embodiment, the deletion comprises a deletion of at most 2 nucleotides.

According to a specific embodiment, the deletion comprises a deletion of one nucleotide.

According to one embodiment, the mutation comprises a point mutation.

According to a specific embodiment, the point mutation comprises a point mutation of about 1-500 nucleotides, about 1-250 nucleotides, about 1-150 nucleotides, about 1-100 nucleotides, about 1-50 nucleotides, about 1-25 nucleotides, about 1-10 nucleotides, about 10-250 nucleotides, about 10-200 nucleotides, about 10-150 nucleotides, about 10-100 nucleotides, about 10-50 nucleotides, about 1-50 nucleotides, about 1-10 nucleotides, about 50-150 nucleotides, about 50-100 nucleotides or about 100-200 nucleotides (as compared to the nucleotide sequence of the wild type component of the caffeine biosynthesis pathway, e.g. XMT/DXMT/MXMT).

According to one embodiment, the point mutation comprises a point mutation in at most 1, 2, 3, 4, 5, 6, 7, 8, 9, 10, 11, 12, 13, 14, 15, 16, 17, 18, 19, 20, 22, 24, 26, 28, 30, 32, 34, 36, 38, 40, 42, 44, 46, 48, 50, 60, 70, 80, 90, 100, 110, 120, 130, 140, 150, 160, 170, 180, 190, 200, 250, 300, 350, 400, 450 or at most 500 nucleotides (as compared to the nucleotide sequence of the wild type component of the caffeine biosynthesis pathway, e.g. XMT/DXMT/MXMT).

According to a specific embodiment, the point mutation comprises a point mutation in at most 200 nucleotides.

According to a specific embodiment, the point mutation comprises a point mutation in at most 150 nucleotides.

According to a specific embodiment, the point mutation comprises a point mutation in at most 100 nucleotides.

According to a specific embodiment, the point mutation comprises a point mutation in at most 50 nucleotides.

According to a specific embodiment, the point mutation comprises a point mutation in at most 25 nucleotides.

According to a specific embodiment, the point mutation comprises a point mutation in at most 20 nucleotides.

According to a specific embodiment, the point mutation comprises a point mutation in at most 15 nucleotides.

According to a specific embodiment, the point mutation comprises a point mutation in at most 10 nucleotides.

According to a specific embodiment, the point mutation comprises a point mutation in at most 5 nucleotides.

According to a specific embodiment, the point mutation comprises a point mutation in at most 2 nucleotides.

According to a specific embodiment, the point mutation comprises a point mutation in one nucleotide.

According to one embodiment, the mutation comprises a combination of any of a deletion, an insertion and/or a point mutation.

According to one embodiment, the mutation comprises nucleotide replacement (e.g. substitution).

According to a specific embodiment, the substitution comprises substitution of about 1-500 nucleotides, 1-450 nucleotides, 1-400 nucleotides, 1-350 nucleotides, 1-300 nucleotides, 1-250 nucleotides, 1-200 nucleotides, 1-150 nucleotides, 1-100 nucleotides, 1-90 nucleotides, 1-80 nucleotides, 1-70 nucleotides, 1-60 nucleotides, 1-50 nucleotides, 1-40 nucleotides, 1-30 nucleotides, 1-20 nucleotides, 1-10 nucleotides, 10-100 nucleotides, 10-90 nucleotides, 10-80 nucleotides, 10-70 nucleotides, 10-60 nucleotides, 10-50 nucleotides, 10-40 nucleotides, 10-30 nucleotides, 10-20 nucleotides, 10-15 nucleotides, 20-30 nucleotides, 20-50 nucleotides, 20-70 nucleotides, 30-40 nucleotides, 30-50 nucleotides, 30-70 nucleotides, 40-50 nucleotides, 40-80 nucleotides, 50-60 nucleotides, 50-70 nucleotides, 50-90 nucleotides, 60-70 nucleotides, 60-80 nucleotides, 70-80 nucleotides, 70-90 nucleotides, 80-90 nucleotides, 90-100 nucleotides, 100-110 nucleotides, 100-120 nucleotides, 100-130 nucleotides, 100-140 nucleotides, 100-150 nucleotides, 100-160 nucleotides, 100-170 nucleotides, 100-180 nucleotides, 100-190 nucleotides, 100-200 nucleotides, 110-120 nucleotides, 120-130 nucleotides, 130-140 nucleotides, 140-150 nucleotides, 160-170 nucleotides, 180-190 nucleotides, 190-200 nucleotides, 200-250 nucleotides, 250-300 nucleotides, 300-350 nucleotides, 350-400 nucleotides, 400-450 nucleotides, or about 450-500 nucleotides (as compared to the nucleotide sequence of the wild type component of the caffeine biosynthesis pathway, e.g. XMT/DXMT/MXMT).

According to one embodiment, the nucleotide swap comprises a nucleotide replacement in at most 1, 2, 3, 4, 5, 6, 7, 8, 9, 10, 11, 12, 13, 14, 15, 16, 17, 18, 19, 20, 22, 24, 26, 28, 30, 32, 34, 36, 38, 40, 42, 44, 46, 48, 50, 60, 70, 80, 90, 100, 110, 120, 130, 140, 150, 160, 170, 180, 190, 200, 250, 300, 350, 400, 450 or at most 500 nucleotides (as compared to the nucleotide sequence of the wild type component of the caffeine biosynthesis pathway, e.g. XMT/DXMT/MXMT).

According to a specific embodiment, the nucleotide substitution comprises a nucleotide replacement in at most 200 nucleotides.

According to a specific embodiment, the nucleotide substitution comprises a nucleotide replacement in at most 150 nucleotides.

According to a specific embodiment, the nucleotide substitution comprises a nucleotide replacement in at most 100 nucleotides.

According to a specific embodiment, the nucleotide substitution comprises a nucleotide replacement in at most 50 nucleotides.

According to a specific embodiment, the nucleotide substitution comprises a nucleotide replacement in at most 25 nucleotides.

According to a specific embodiment, the nucleotide substitution comprises a nucleotide replacement in at most 20 nucleotides.

According to a specific embodiment, the nucleotide substitution comprises a nucleotide replacement in at most 15 nucleotides.

According to a specific embodiment, the nucleotide substitution comprises a nucleotide replacement in at most 10 nucleotides.

According to a specific embodiment, the nucleotide substitution comprises a nucleotide replacement in at most 5 nucleotides.

According to a specific embodiment, the nucleotide substitution comprises a nucleotide replacement in at most 2 nucleotides.

According to a specific embodiment, the nucleotide substitution comprises a nucleotide replacement in one nucleotide.

According to a specific embodiment, the genome editing event comprises introduction of foreign DNA into a genome of the coffee plant (e.g. insertion or substitution mutation) that could otherwise be introduced into the plant by traditional breeding e.g. from a second plant (e.g. by crossing).

According to a specific embodiment, the genome editing event does not comprise introduction of foreign DNA into a genome of the coffee plant (e.g. insertion or substitution mutation) that could be introduced through traditional breeding (e.g. by crossing).

Methods for detecting sequence alteration are well known in the art and include, but not limited to, DNA sequencing (e.g., next generation sequencing), electrophoresis, an enzyme-based mismatch detection assay and a hybridization assay such as PCR, RT-PCR, RNase protection, in-situ hybridization, primer extension, Southern blot, Northern Blot and dot blot analysis. Various methods used for detection of single nucleotide polymorphisms (SNPs) can also be used, such as PCR based T7 endonuclease, Hetroduplex and Sanger sequencing.

Another method of validating the presence of a DNA editing event e.g., Indels comprises a mismatch cleavage assay that makes use of a structure selective enzyme (e.g. endonuclease) that recognizes and cleaves mismatched DNA.

The mismatch cleavage assay is a simple and cost-effective method for the detection of indels and is therefore the typical procedure to detect mutations induced by genome editing. The assay uses enzymes that cleave heteroduplex DNA at mismatches and extrahelical loops formed by multiple nucleotides, yielding two or more smaller fragments. A PCR product of approximately 300-1000 bp is generated with the predicted nuclease cleavage site off-center so that the resulting fragments are dissimilar in size and can easily be resolved by conventional gel electrophoresis or high-performance liquid chromatography (HPLC). End-labeled digestion products can also be analyzed by automated gel or capillary electrophoresis. The frequency of indels at the locus can be estimated by measuring the integrated intensities of the PCR amplicon and cleaved DNA bands. The digestion step takes 15-60 min, and when the DNA preparation and PCR steps are added the entire assays can be completed in <3 h.

Two alternative enzymes are typically used in this assay. T7 endonuclease 1 (T7E1) is a resolvase that recognizes and cleaves imperfectly matched DNA at the first, second or third phosphodiester bond upstream of the mismatch. The sensitivity of a T7E1-based assay is 0.5-5%. In contrast, Surveyor™ nuclease (Transgenomic Inc., Omaha, Nebr., USA) is a member of the CEL family of mismatch-specific nucleases derived from celery. It recognizes and cleaves mismatches due to the presence of single nucleotide polymorphisms (SNPs) or small indels, cleaving both DNA strands downstream of the mismatch. It can detect indels of up to 12 nt and is sensitive to mutations present at frequencies as low as approximately 3%, i.e. 1 in 32 copies.

Yet another method of validating the presence of an editing even comprises the high-resolution melting analysis.

High-resolution melting analysis (HRMA) involves the amplification of a DNA sequence spanning the genomic target (90-200 bp) by real-time PCR with the incorporation of a fluorescent dye, followed by melt curve analysis of the amplicons. HRMA is based on the loss of fluorescence when intercalating dyes are released from double-stranded DNA during thermal denaturation. It records the temperature-dependent denaturation profile of amplicons and detects whether the melting process involves one or more molecular species.

Yet another method is the heteroduplex mobility assay. Mutations can also be detected by analyzing re-hybridized PCR fragments directly by native polyacrylamide gel electrophoresis (PAGE). This method takes advantage of the differential migration of heteroduplex and homoduplex DNA in polyacrylamide gels. The angle between matched and mismatched DNA strands caused by an indel means that heteroduplex DNA migrates at a significantly slower rate than homoduplex DNA under native conditions, and they can easily be distinguished based on their mobility.

Fragments of 140-170 bp can be separated in a 15% polyacrylamide gel. The sensitivity of such assays can approach 0.5% under optimal conditions, which is similar to T7E1. After reannealing the PCR products, the electrophoresis component of the assay takes approximately 2 hours.

Other methods of validating the presence of editing events are described in length in Zischewski 2017 Biotechnol. Advances 1(1):95-104, incorporated herein by reference.

Coffee plants can be diploid or polyploid e.g., tetraploid, as described in e.g. Tran, Hue T M et al. “Use of a draft genome of coffee (Coffea arabica) to identify SNPs associated with caffeine content”, Plant biotechnology Journal (2018) 16(10): 1756-1766. doi:10.1111/pbi.12912, incorporated herein by reference. Accordingly, it will be appreciated that positive clones can be homozygous (i.e. an edit occurs at all alleles) or heterozygous (i.e. an edit occurs in at least one of the alleles, e.g. in one, two, three, four, five, six, seven or more alleles) for the DNA editing event. In cases of heterozygous form, different alleles may carry different editing events. Additionally, in a heterozygous form, not all of the alleles may carry the event (same or different edit event). In cases of homozygous form, all alleles may carry the same editing event. The skilled artisan will select the clone for further culturing/regeneration and crossing according to the intended use.

Clones exhibiting the presence of a DNA editing event as desired are further analyzed for the presence of the DNA editing agent. Namely, loss of DNA sequences encoding for the DNA editing agent, pointing to the transient nature of the method.

This can be done by analyzing the expression of the DNA editing agent (e.g., at the mRNA, protein) e.g., by fluorescent detection of GFP or q-PCR.

Alternatively or additionally, the cells are analyzed for the presence of the nucleic acid construct as described herein or portions thereof e.g., nucleic acid sequence encoding the reporter polypeptide or the DNA editing agent.

Clones showing no DNA encoding the fluorescent reporter or DNA editing agent (e.g., as affirmed by fluorescent microscopy, q-PCR and or any other method such as Southern blot, PCR, sequencing) yet comprising the DNA editing event(s) [mutation(s)] as desired are isolated for further processing.

These clones can therefore be stored (e.g., cryopreserved).

Alternatively, cells (e.g., protoplasts) may be regenerated into whole plants first by growing into a group of plant cells that develops into a callus and then by regeneration of shoots (caulogenesis) from the callus using plant tissue culture methods. Growth of protoplasts into callus and regeneration of shoots requires the proper balance of plant growth regulators in the tissue culture medium that must be customized for each species of plant.

Protoplasts may also be used for plant breeding, using a technique called protoplast fusion. Protoplasts from different species are induced to fuse by using an electric field or a solution of polyethylene glycol. This technique may be used to generate somatic hybrids in tissue culture.

Methods of protoplast regeneration are well known in the art. Several factors affect the isolation, culture, and regeneration of protoplasts, namely the genotype, the donor tissue and its pre-treatment, the enzyme treatment for protoplast isolation, the method of protoplast culture, the culture, the culture medium, and the physical environment. For a thorough review see Maheshwari et al. 1986 Differentiation of Protoplasts and of Transformed Plant Cells: 3-36. Springer-Verlag, Berlin, incorporated herein by reference.

The regenerated plants can be subjected to further breeding, selfing, crossing, backcrossing and selection as the skilled artisan sees fit.

The phenotype of the final lines, plants or intermediate breeding products can be analyzed such as by determining the sequence of the methyltransferase gene (e.g. XMT, MXMT and/or DXMT), expression thereof in the mRNA or protein level, activity of the protein and/or analyzing the properties of the coffee been (e.g. reduced caffeine level).

As is illustrated herein and in the Examples section which follows. The present inventors were able to transform coffee with a genome editing agent, while avoiding stable transgenesis.

Hence the present methodology allows genome editing without integration of a selectable or screenable reporter.

Thus, embodiments of the invention further relate to plants, plant parts (e.g. beans), plant cells and processed product of plants comprising the gene editing event(s) generated according to the present teachings.

According to one aspect of the present invention there is provided a coffee plant comprising a genome comprising a loss of function mutation in a nucleic acid sequence encoding at least one component of a caffeine biosynthesis pathway.

According to one embodiment of the present invention there is provided a coffee plant generated according to the methods described herein.

According to one embodiment, the coffee plant or part thereof of some embodiments of the invention comprises reduced caffeine content as compared to that of a coffee plant of the same genetic background and developmental stage and growth conditions devoid of the loss of function mutation. According to a specific embodiment, the reduced caffeine content is by at least 5%, 10%, 20%, 30%, 40%, 50%, 60%, 70%, 80%, 90%, 95%, 99% or even more as compared to that of a coffee plant of the same genetic background and developmental stage and growth conditions devoid of the loss of function mutation.

According to one embodiment, the coffee plant of some embodiments of the invention comprises at least about 5% reduction in caffeine as compared to that of a coffee plant of the same genetic background and developmental stage and growth conditions devoid of the loss of function mutation.

According to one embodiment, the coffee plant of some embodiments of the invention comprises at least about 10% reduction in caffeine as compared to that of a coffee plant of the same genetic background and developmental stage and growth conditions devoid of the loss of function mutation.

According to one embodiment, the coffee plant of some embodiments of the invention is non-transgenic (non-GMO).

According to one embodiment, the coffee plant of some embodiments of the invention is transgenic (GMO).

The present teachings also relate to parts of the plants as described herein or processed products thereof.

According to a specific embodiment, the plant part is a bean.

According to another specific embodiment, the bean is dry.

According to some embodiments there is provided a method of producing coffee beans with reduced caffeine content, the method comprising:

(a) growing the plant of some embodiments of the invention; and

(b) harvesting beans from the plant.

According to further embodiments there is provided a method of producing coffee with reduced caffeine content, the method comprising subjecting beans of some embodiments of the invention to extraction, dehydration and optionally roasting.

Any method known in the art for harvesting coffee beans (from coffee plants) may be used in accordance with the present invention. For example, coffee cherries (i.e. coffee fruit comprising the beans) may be picked by strip picking (wherein the coffee cherries are stripped off of the branch at one time, either by machine or by hand) or by selective picking (wherein only the ripe cherries are harvested, and are picked individually by hand).

Furthermore, any method known in the art for processing coffee beans may be used in accordance with the present invention.

According to one embodiment, coffee beans are processed by “wet processing” wherein the flesh/skin of the cherries is separated from the beans and then the beans are fermented—soaked in water for e.g. about two days. The beans may then be washed and dried in e.g. the sun, or, in the case of commercial manufacturers, in drying machines.

According to one embodiment, coffee beans are processed by “dry processing” wherein twigs and other foreign objects are separated from the cherries, and the cherries are spread out in the sun on e.g. concrete or brick for e.g. 2-3 weeks (where fermentation occurs), turned regularly for even drying.

It will be appreciated that regardless of the processing method used (e.g. “wet processing” or “dry processing”), the flesh/skin of the cherries is typically removed prior to the start of fermentation.

According to one embodiment, after processing has taken place, the husks are removed (from the beans) and the beans are roasted.

According to one embodiment, there is provided coffee of the beans of some embodiments of the invention.

Processed coffee compositions of some embodiments can be in the form of a coffee powder to be extracted or brewed or a soluble coffee powder. Thus, it can be coarse-ground coffee, filter coffee or instant coffee. On the other hand, the coffee composition of the invention can also comprise whole roasted coffee beans.

According to one embodiment, the coffee is in a powder form.

According to one embodiment, the coffee is in a granulated form.

Further embodiments of the invention relate to a coffee beverage comprising the coffee composition and water. Such a coffee beverage can be prepared with methods known to a person skilled in the art, such as by extracting with water, brewing in water or soaking the coffee composition of the invention in water.

The coffee beverage of the invention can also comprise other substances, such as natural or artificial flavoring substances, milk products, alcohol, foaming agents, natural or artificial sweetening agents, and the like.

The coffee compositions of some embodiments are suitable for use in drinks such as, but not limited to, Americano, Cappuccino, Cafe Late, Expresso, Macchiato, Black, Flat white, Affogato, Mochachino, Irish Coffee and Mocha.

Further embodiments of the present invention relate to use of the coffee compositions for producing ready to drink beverages, creamers, coffee mixes, cocoa malt beverages, as well as for producing chocolate, bakery or culinary products.

The coffee compositions of the invention may be packed in capsules to be used in beverage dispensers. Additionally or alternatively, the coffee compositions of the invention may be packed in paper, fabric or plastic bags, preferably such that the dryness and freshness of the coffee is maintained.

As used herein the term “about” refers to ±10%.

The terms “comprises”, “comprising”, “includes”, “including”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

As used herein, the term “treating” includes abrogating, substantially inhibiting, slowing or reversing the progression of a condition, substantially ameliorating clinical or aesthetical symptoms of a condition or substantially preventing the appearance of clinical or aesthetical symptoms of a condition.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples.

It is understood that any Sequence Identification Number (SEQ ID NO) disclosed in the instant application can refer to either a DNA sequence or a RNA sequence, depending on the context where that SEQ ID NO is mentioned, even if that SEQ ID NO is expressed only in a DNA sequence format or a RNA sequence format. For example, SEQ ID NO: 1 is expressed in a DNA sequence format (e.g., reciting T for thymine), but it can refer to either a DNA sequence that corresponds to an MXMT nucleic acid sequence, or the RNA sequence of an RNA molecule nucleic acid sequence. Similarly, though some sequences are expressed in a RNA sequence format (e.g., reciting U for uracil), depending on the actual type of molecule being described, it can refer to either the sequence of a RNA molecule comprising a dsRNA, or the sequence of a DNA molecule that corresponds to the RNA sequence shown. In any event, both DNA and RNA molecules having the sequences disclosed with any substitutes are envisioned.

### EXAMPLES

Reference is now made to the following examples, which together with the above descriptions, illustrate the invention in a non-limiting fashion.

Generally, the nomenclature used herein and the laboratory procedures utilized in the present invention include molecular, biochemical, microbiological and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Md. (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Current Protocols in Immunology” Volumes I-III Coligan J. E., ed. (1994); Stites et al. (eds), “Basic and Clinical Immunology” (8th Edition), Appleton & Lange, Norwalk, Conn. (1994); Mishell and Shiigi (eds), “Selected Methods in Cellular Immunology”, W. H. Freeman and Co., New York (1980); available immunoassays are extensively described in the patent and scientific literature, see, for example, U.S. Pat. Nos. 3,791,932; 3,839,153; 3,850,752; 3,850,578; 3,853,987; 3,867,517; 3,879,262; 3,901,654; 3,935,074; 3,984,533; 3,996,345; 4,034,074; 4,098,876; 4,879,219; 5,011,771 and 5,281,521; “Oligonucleotide Synthesis” Gait, M. J., ed. (1984); “Nucleic Acid Hybridization” Hames, B. D., and Higgins S. J., eds. (1985); “Transcription and Translation” Hames, B. D., and Higgins S. J., Eds. (1984); “Animal Cell Culture” Freshney, R. I., ed. (1986); “Immobilized Cells and Enzymes” IRL Press, (1986); “A Practical Guide to Molecular Cloning” Perbal, B., (1984) and “Methods in Enzymology” Vol. 1-317, Academic Press; “PCR Protocols: A Guide To Methods And Applications”, Academic Press, San Diego, Calif. (1990); Marshak et al., “Strategies for Protein Purification and Characterization—A Laboratory Course Manual” CSHL Press (1996); all of which are incorporated by reference as if fully set forth herein. Other general references are provided throughout this document. The procedures therein are believed to be well known in the art and are provided for the convenience of the reader. All the information contained therein is incorporated herein by reference.

### General Materials and Experimental Procedures

Embryogenic Callus and Cell Suspension Generation and Maintenance

Embryonic calli were obtained as previously described [Etienne, H., Somatic embryogenesis protocol: coffee (Coffea arabica L. and C. canephora P.), in Protocol for somatic embryogenesis in woody plants. 2005, Springer. p. 167-1795]. Briefly, young leaves were surface sterilized, cut into 1 cm² pieces and placed on half strength semi solid MS medium supplemented with 2.26 μM 2,4-dichlorophenoxyacetic acid (2,4-D), 4.92 μM indole-3-butyric acid (IBA) and 9.84 μM isopentenyladenine (iP) for one month. Explants were then transferred to half strength semisolid MS medium containing 4.52 μM 2,4-D and 17.76 μM 6-benzylaminopurine (6-BAP) for 6 to 8 months until regeneration of embryogenic calli. Embryogenic calli were maintained on MS media supplemented with 5 μM 6-BAP.

Cell suspension cultures were generated from embryogenic calli as previously described [Acuna, J. R. and M. de Pena, Plant Cell Reports (1991). 10(6): 345-348]. Embryogenic calli (30 g/l) were placed in liquid MS medium supplemented with 13.32 μM 6-BAP. Flasks were placed in a shaking incubator (110 rpm) at 28° C. The cell suspension was subcultured/passaged every two to four weeks until fully established. Cell suspension cultures were maintained in liquid MS medium with 4.44 μM 6-BAP.

Target Genes

The target genes in cultivar Coffea canephora (Robusta coffee) are the genes which encode methyltransferases: xanthosine methyltransferase (XMT), 7-methyxanthine methyltrasferase (MXMT or theobromine synthase), and 3,7-dimethylxanthine methyltransferase (DXMT or caffeine synthase).

TABLE 1A
Target genes

| Gene name | Accession number |
|---|---|
| CaDXMT1 | AB084125.1 |
| CcDXMT1 | DQ422955 |
| CaMXMT1 | AB048794.1 |
| CaMXMT2 | AB084126 |
| CaXMT1 | AB048793 |
| CcXMT1 | DQ422954 |

Of note, Ca: Coffea arabica;
Cc: Coffea canephora.

sgRNAs Design

sgRNA sequences are designed according to two separate strategies. The first strategy involves targeting the XMT gene (Cc09_g06970) directly using two crRNA. The second strategy exploits the fact that the genes share homology so therefore crRNA was designed to target all the expressed copies of decaffeination genes (also termed decaff coffee genes) in the pathway.

crRNA sequences were designed using the online CRISPR RGEN Tool (www(dot)rgenome.net/) and the best pair was chosen depending on uniqueness to the target sequences dependent on strategy.

Each gene from each coffee variety was sequenced and aligned to ensure crRNA targets do not contain SNPs which would inhibit sgRNA binding. The sgRNA sequences were designed for work with the 4 lines of Coffea canephora, termed 06, 07, 09 & 23.

sgRNAs sequences

| | |
|---|---|
| sgRNA #6 | AAAACCGAATTGAAATCATT (SEQ ID NO: 51) |
| sgRNA #7 | TGCCTAATAGGGGCAATGCC (SEQ ID NO: 52) |
| sgRNA #11 | TTCAAGGACAGGTTTCACCT (SEQ ID NO: 53) |
| sgRNA #12 | CAACAAGTGCATTAAAGTTG (SEQ ID NO: 54) |
| sgRNA #13 | AAAGAAAATGGACGCAAAAT (SEQ ID NO: 55) |
| sgRNA #14 | AAAAAATGCATGGACTCCTC (SEQ ID NO: 56) |
| sgRNA #37 | CGTATGCATTGTTCAAGGAA (SEQ ID NO: 57) |
| sgRNA #38 | AAAGAAAATGGACGCAAGAT (SEQ ID NO: 58) |

sgRNA Cloning

Plasmids utilized were composed of transcriptional units comprising of (i), eGFP driven by the CaMV35s promoter; (ii), Cas9 driven by the CaMV35s promoter; and (iii), AtU6 promoters driving sgRNAs. A binary vector can be used such as pCAMBIA or pRI-201-AN DNA.

Protoplasts Isolation

Protoplasts were isolated by incubating plant material (e.g. leaves or calli) in a digestion solution (1% cellulase, 0.5% macerozyme, 0.5% driselase, 0.4 M mannitol, 154 mM NaCl, 20 mM KCl, 20 mM MES pH 5.6, 10 mM CaCl2) for 4-24 hours at room temperature and gentle shaking. After digestion, remaining plant material was washed with W5 solution (154 mM NaCl, 125 mM CaCl2, 5 mM KCl, 2 mM MES pH 5.6) and protoplasts suspension was filtered through a 40 μm strainer. After centrifugation at 80 g for 3 minutes at room temperature, protoplasts were resuspended in 2 ml W5 buffer and precipitated by gravity in ice. The final protoplast pellet was resuspended in 2 ml of MMG (0.4 M mannitol, 15 mM MagCl2, 4 mM MES pH 5.6) and protoplast concentration was determined using a hemocytometer. Protoplasts viability was estimated using

Trypan Blue Staining.

Polyethylene glycol (PEG)-mediated plasmid transfection PEG-transfection of coffee protoplasts is effected using a modified version of the strategy reported by Wang et al, (2015) [Wang, H., et al., Scientia Horticulturae (2015) 191: 82-89]. Protoplasts are resuspended to a density of 2-5×10⁶ protoplasts/ml in MMG solution. 100-200 μl of protoplast suspension is added to a tube containing the plasmid. The plasmid:protoplast ratio greatly affects transformation efficiency therefore a range of plasmid concentrations in protoplast suspension, 5-300 μg/μ1, are assayed. PEG solution (100-200 μl) is added to the mixture and incubated at 23° C. for various lengths of time ranging from 10-60 minutes. PEG4000 concentration is optimized, a range of 20-80% PEG4000 in 200-400 mM mannitol, 100-500 mM CaCl₂ solution is assayed. The protoplasts are then washed in W5 and centrifuged at 80 g for 3 min, prior resuspension in 1 ml W5 and incubated in the dark at 23° C. After incubation for 24-72 h fluorescence is detected by microscopy.

Cells/Tissue Bombardment

Particle bombardment is used as a means to introduce DNA into plant cells using high-velocity microprojectiles. The protocol described previously is utilized (Hibberd Laboratory, Department of Plant Sciences, University of Cambridge) using C. canephora leaves and calli as starting material. Briefly, calli or surfaced-sterilized leaves are plated onto medium containing mannitol for osmotic treatment. Meanwhile, DNA-coated gold particles are prepared by weighing 40 mg of 1.0 um diameter gold and mixing it with 100% ethanol in a low-binding Eppendorf tube at 4° C. A centrifugation and washing step of the gold particles is followed by coating with DNA: addition of 45 μl of plasmid (1000 ng/μl), vortexing and rotation. Next, a mix of spermidine and CaCl₂ is prepared, which is subsequently added to the DNA-coated gold particles. After cooling down on ice, the DNA-coated gold particles mix is washed again in ethanol and left in fresh 100% ethanol ready for bombardment. The Biolistic PDS-1000/He Instrument (Bio-Rad) is used for bombardment. 80-450 psi rupture disks are placed into isopropanol and sterilized macrocarriers (chamber and all components are sterilized with 70% ethanol). Next, the DNA-coated gold particles mixture is placed onto the centre of each microcarrier, the ethanol is allowed to evaporate and all components are assembled for bombardment. The vacuum pressure is set, helium valve is opened and calli or leaves are bombarded. After bombardment, calli or leaves are passed to post-bombardment medium to reduce osmotic potential and are incubated in the dark to allow cell repair.

FACS Sorting of Fluorescent Protein-Expressing Cells

48 hrs after plasmid/RNA delivery, cells were collected and sorted for fluorescent protein expression using a flow cytometer in order to enrich for GFP/Editing agent expressing cells [as previously described in Chiang et al., Sci Rep (2016). 6: 24356]. This enrichment step allows bypassing antibiotic selection and collection of only cells transiently expressing the fluorescent protein, Cas9 and the sgRNA. These cells could be further tested for editing of the target gene by non-homologues end joining (NHEJ) and loss of the corresponding gene expression.

Screening for Gene Modification and Absence of CRISPR System DNA

From each colony, DNA was extracted from an aliquot of RFP-sorted protoplasts (optional step) or bombarded-derived colonies and a PCR reaction was performed with primers flanking the targeted gene. Measures were taken to sample the colony as positive—colonies that were later used to regenerate the plant. A control reaction subjected to the same method but without Cas9-sgRNA was included and considered as wild type (WT). The PCR products were then separated on an agarose gel to detect any changes in the product size compared to the WT. The PCR reaction products that vary from the WT products were cloned into pBLUNT (Invitrogen). In addition, sequencing was used to verify the editing event. The resulting colonies were picked, plasmids were isolated and sequenced to determine the nature of the mutations. Clones (colonies or calli) harboring mutations that were predicted to result in domain-alteration or complete loss of the corresponding protein were chosen for whole genome sequencing to validate that they were free from the CRISPR system DNA/RNA and to detect the mutations at the genomic DNA level.

Plant Regeneration

Clones that were sequenced and predicted to have lost the expression of the target genes and found to be free of the CRISPR system DNA/RNA were propagated for generation in large quantities and in parallel were differentiated to generate seedlings from which functional assay is performed to test the desired trait.

In short, transfected protoplasts were plated at high density on cellulose membranes on feeder plates to allow for colony formation for about 15 weeks. During this time, protoplasts were fed with liquid media (B5 media plus vitamins, 92 g/L glucose) weekly. After 15 weeks, protocolonies (microcalli) were transferred to proliferation medium (half strength MS+B5 vitamins, +30 g/L sucrose). Next, proliferating calli were transferred to regeneration media (half strength MS+B5 vitamins, 20 g/l sucrose) for embryo development and germination. 3-4 weeks later, germinating embryos are ready to be transferred to solid medium for seedlings elongation.

TABLE 1B
Suggested coffee target genes and number designed and tested of sgRNAs

| | | Overall | |
|---|---|---|---|
| | | copies | Selected |
| | | per | number |
| | Gene versions | diploid | of |
| Gene name | (not alleles) | genome | sgRNAs |
| Decaf-XMT | Cc09_g06970 | 2 | 6 |
| Decaf-MXMT | Cc00_g24720 | 2 | 4 |
| Decaf-DXMT | Cc01_g00720 | 2 | 2 |
| Decaf- | Cc09_g06950 | 2 | 3 |
| XMT/MXMT/DXMT | Cc09_g06960 | 2 | 4 |

TABLE 1C
Additional sgRNAs designed to target the candidate genes

| | | Additional sgRNAs |
|---|---|---|
| Gene Name | Locus ID | (Unique target sequences) |
| XMT | Cc09_g06970 | 1-TTTGCACAATTAATCATTAAGGG |
| | | (SEQ ID NO: 59) |
| | | 2-CAAGAAGTCCTGCGGATGAATGG |
| | | (SEQ ID NO: 60) |
| | | 3-ACTTGTACATAAATCAAATTGGG |
| | | (SEQ ID NO: 61) |
| | | 4-CAAATTGGGACTGCCAAAGAAGG |
| | | (SEQ ID NO: 62) |
| MXMT | Cc00_g24720 | 5-GAAGTCCTGCATATGAATGAAGG |
| | | (SEQ ID NO: 63) |
| | | 6-GACGGGCGGACGACATCCTTTGG |
| | | (SEQ ID NO: 64) |
| | | 7-TTGGTGATTGAATTGGGGATTGG |
| | | (SEQ ID NO: 65) |
| | | 8-GGGAGTATTTACTCTTCCAAAGG |
| | | (SEQ ID NO: 66) |
| DXMT | Cc01_g00720 | 9-TCAACAAGTGCTTTAAAGTTGGG |
| | | (SEQ ID NO: 67) |
| | | 10-TGCTTTAAAGTTGGGGATTTGGG |
| | | (SEQ ID NO: 68) |
| | | 11-AAAATAGGATCGTGCCTGATAGG |
| | | (SEQ ID NO: 69) |
| | | 12-CGAACTGTTGAAAATGTGTTTGG |
| | | (SEQ ID NO: 70) |
| XMT/MXMT/ | Cc09_g06950 | 13-CCTCGGGGAAGAGTCTGCCGTGG |
| DXMT | | (SEQ ID NO: 71) |
| | | 14-ACTTTGTACAGTGTCCCGAACGG |
| | | (SEQ ID NO: 72) |
| | | 15-ATTAGAACGTCCCACCATTCAGG |
| | | (SEQ ID NO: 73) |
| | | 16-ATGCGACGGCCCGAATACCATGG |
| | | (SEQ ID NO: 74) |
| XMT/MXMT/ | Cc09_g06960 | 17-CATTCGGAAGAGTTGCTTTCAGG |
| DXMT | | (SEQ ID NO: 75) |
| | | 18-GTCTATGGTATTCAGGCCATCGG |
| | | (SEQ ID NO: 76) |
| | | 19-AGCGGATTGGTGACTGAACTGGG |
| | | (SEQ ID NO: 77) |
| | | 20-TCGGAAGAGTTGCTTTCAGGTGG |
| | | (SEQ ID NO: 78) |

### Example 1

### Pipeline Used to Identify Caffeine Biosynthesis Genes

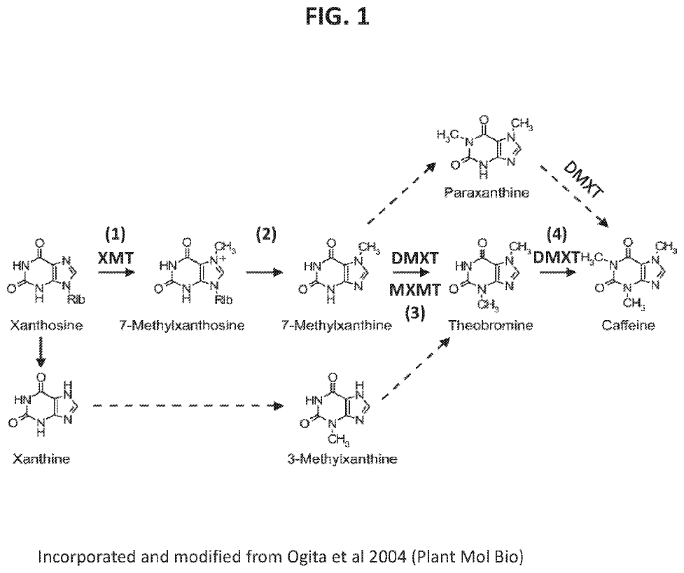

To reduce caffeine levels in Robusta coffee plants, genes associated with caffeine biosynthesis, including XMT, MXMT and DXMT (FIG. 1), were identified by retrieving homologous sequences from characterized pathways in model or crop species. The process involves a series of sequential steps for comparative analysis of DNA and protein sequences that aim at reconstructing the evolutionary history of genes through phylogenetic analysis, filtering candidates by validating their expression in general and target tissue, and sequencing of candidate genes to ensure appropriate sgRNA design (to avoid mismatches). This procedure allowed the selection of genes, the identification of optimized target regions for knockout (conserved and potentially catalytic domains), and the design of appropriate sgRNAs. This pipeline is based on the assumption that homologous proteins with a common ancestor may have a similar function and by doing a phylogenetic reconstruction, gene families are established and assessed for functional diversity in the evolutionary context. This is particularly important for plant species that have undergone large-scale genome duplications and for expanded gene families. Nevertheless, paralogs within a gene family do not necessarily have the same function and part of the process is to target a selection of genes within a family either individually or as a group to also account for redundancy.

### Example 2

### Identifying and Targeting Caffeine Biosynthesis Genes

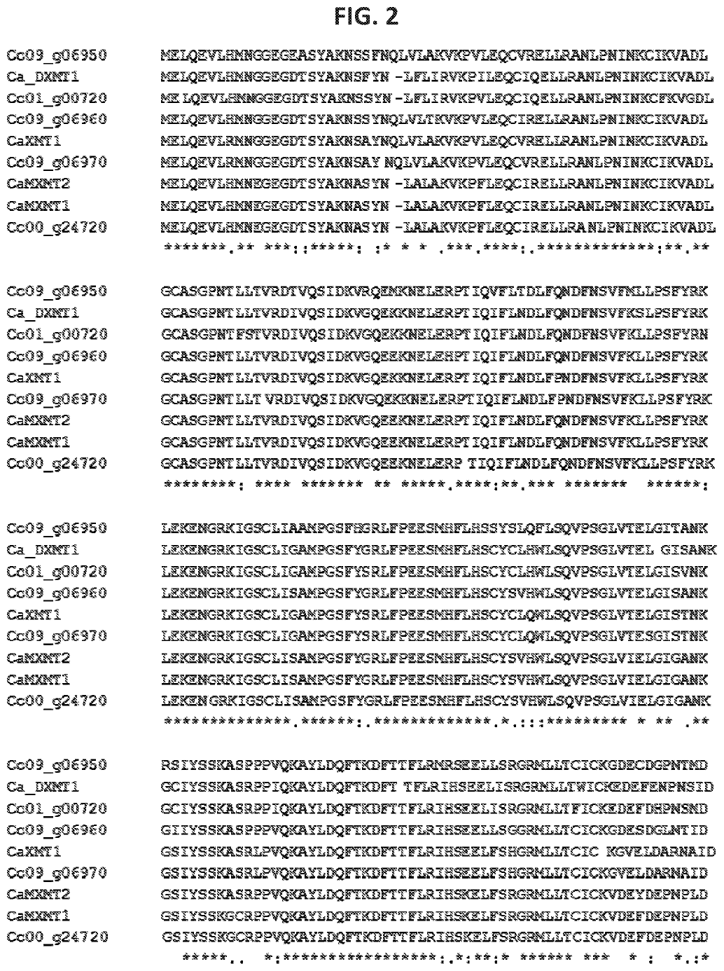

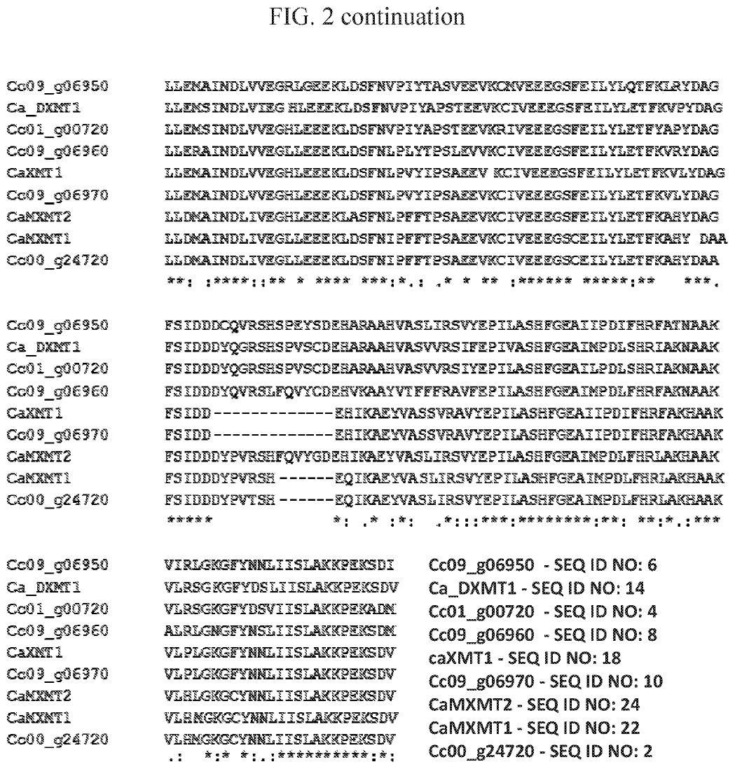

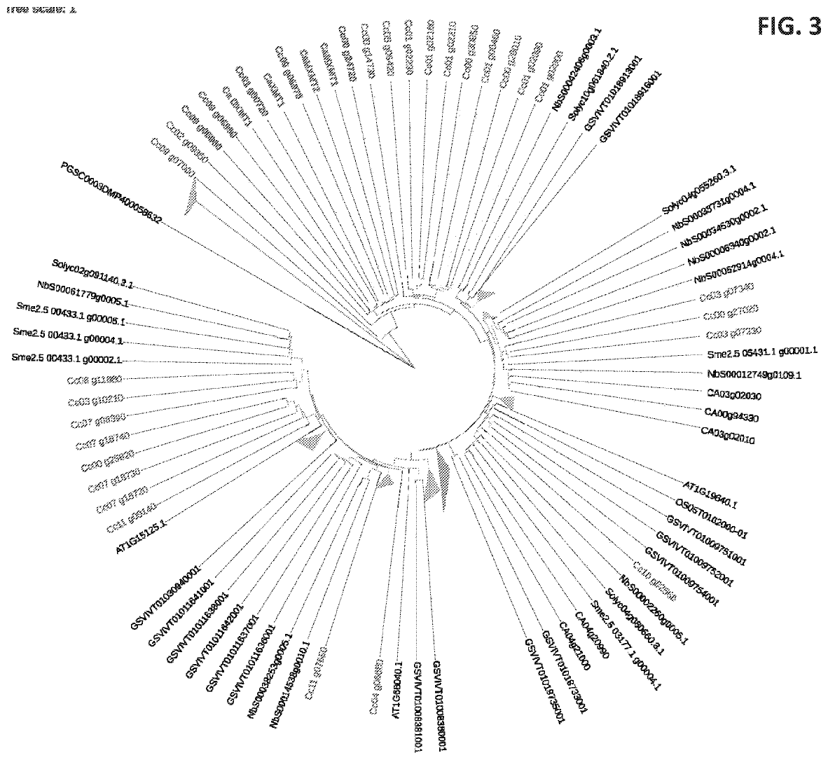

As mentioned, synthesis of the secondary metabolite caffeine involves three methylation reactions to convert xanthosine to 7-methylxanthine to theobromine to caffeine. The key enzymes along this biosynthetic pathway are XMT, MXMT and DXMT, which have been extensively studied and proven to be involved in caffeine production by reconstituting the synthetic pathway in vitro and by expression of the coffee genes in a heterologous system Ogita et al. (2005) supra and Uefuji et al. [Uefuji et al., Plant Molecular Biology (2005) 59:221-227]. Whole-genome analysis of Coffea canephora revealed that several genes involved in secondary metabolite biosynthesis had undergone gene-family expansions, including N-methyltransferases [Denoeud et al., Science (2014) 345(6201)]. This study also indicated that the N-methyltransferases family clustered 23 genes in coffee but had no obvious clusters in other plants species such as Arabidopsis (Denoeud et al. (2014) supra). In order to identify the genes within the coffee genome, which encode putative functional N-methyltransferases, homologous sequences from the characterized caffeine biosynthesis pathway were identified (FIG. 3 and Table 2). Protein alignment showed that the selected genes share around 80-99% similarity (FIG. 2).

TABLE 2
Selected genes and the corresponding closest homolog in C. canephora

| | Gene ID | |
|---|---|---|
| Query gene ID | (C. canephora) | SEQ ID NO: |
| XMT | Cc09_g06970 | 17 |
| (AB048793) | | |
| MXMT | Cc00_g24720 | 21; 23 |
| (AB048794); (AB084126) | | |
| DXMT | Cc01_g00720 | 13 |
| (AB084125) | | |
| XMT/MXMT/DXMT | Cc09_g06950 | 17; 21; 23; 13 |
| (AB048793); (AB048794); | Cc09_g06960 | 17; 21; 23; 13 |
| (AB084126); (AB084125) | | |

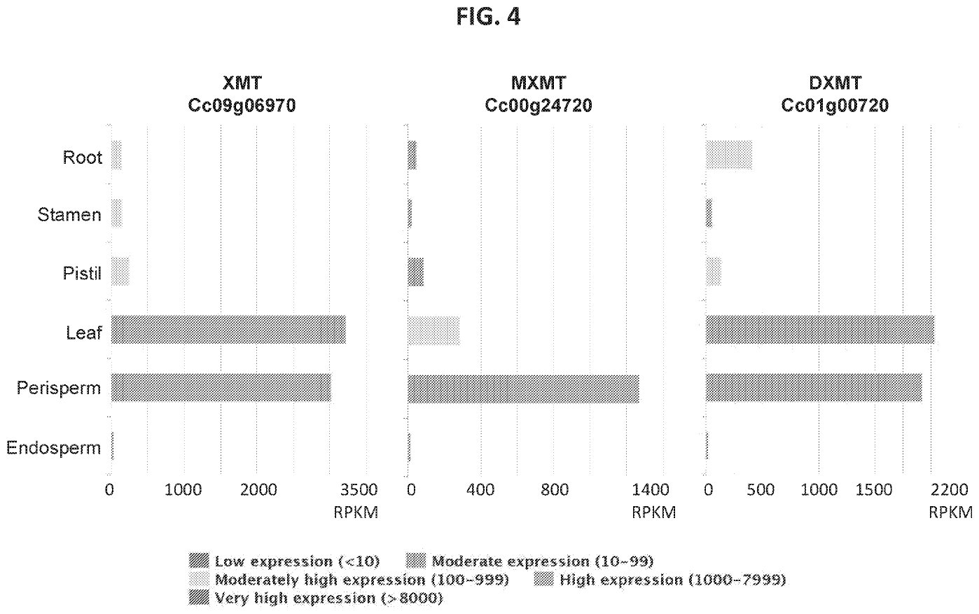

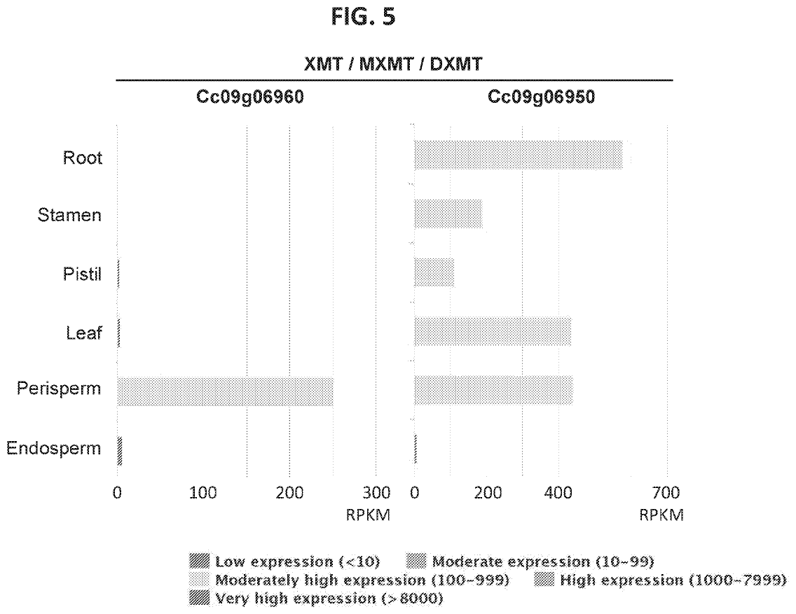

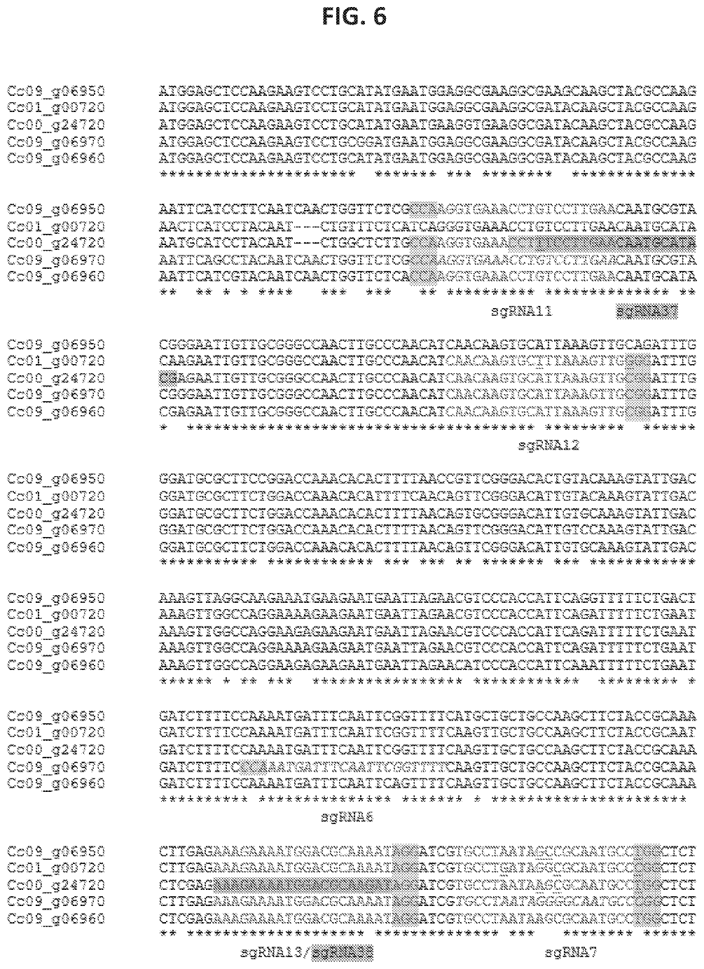

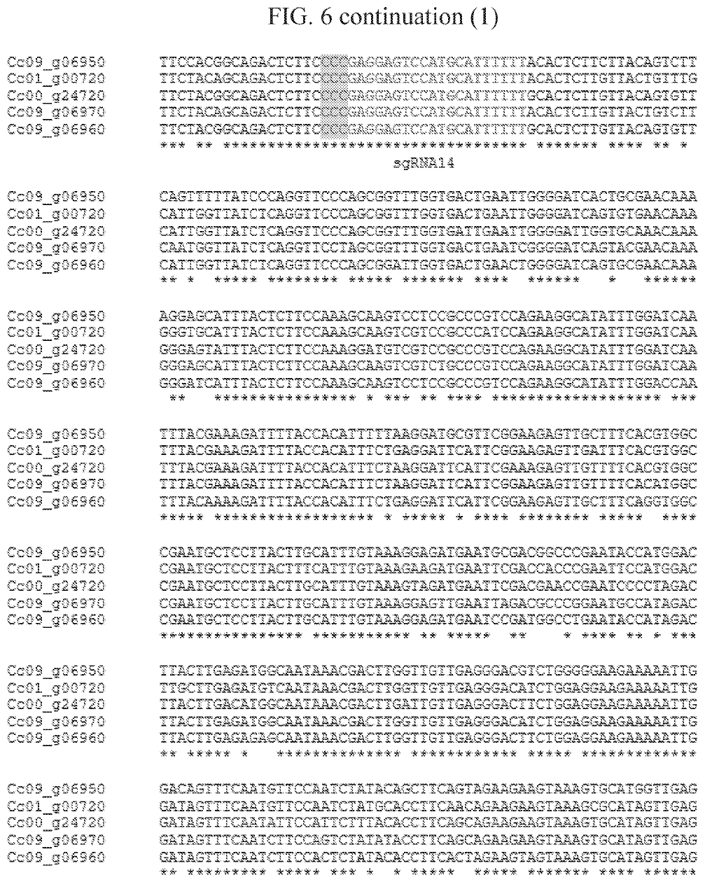

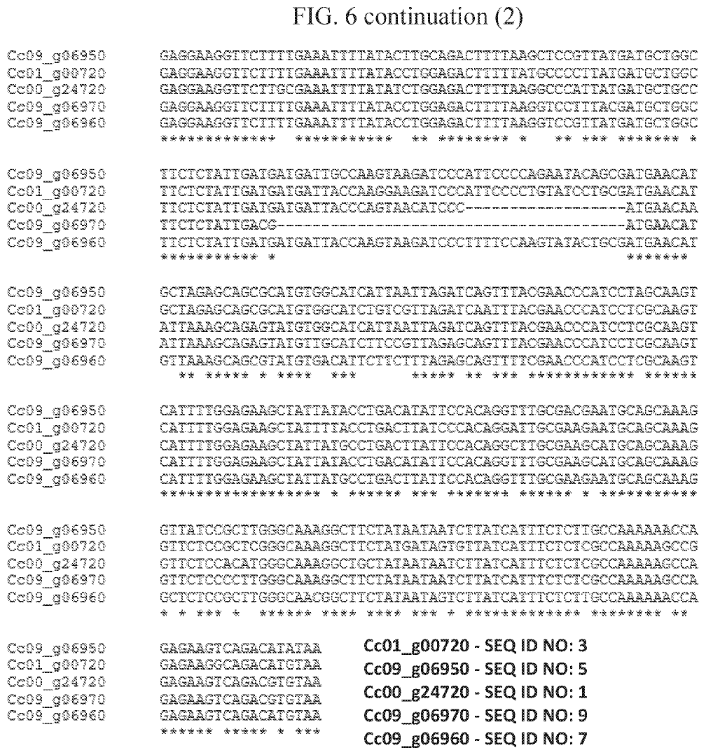

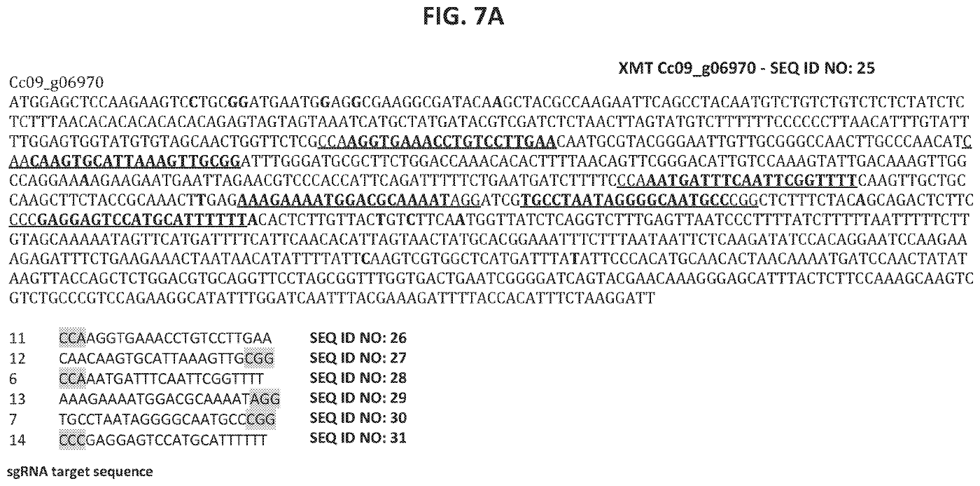

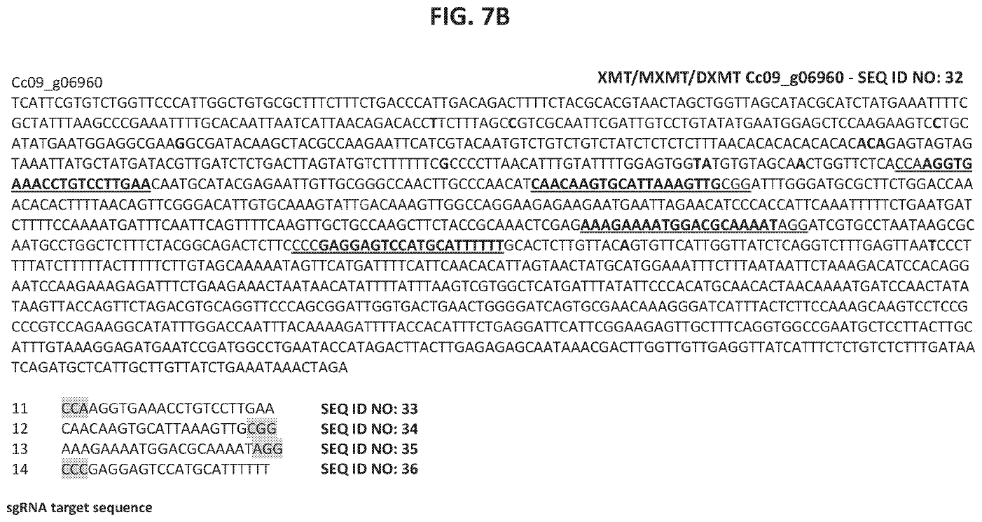

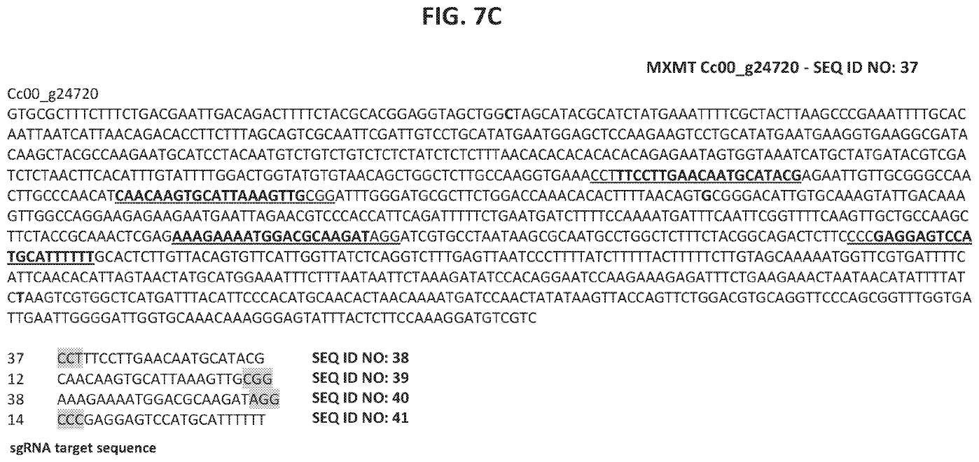

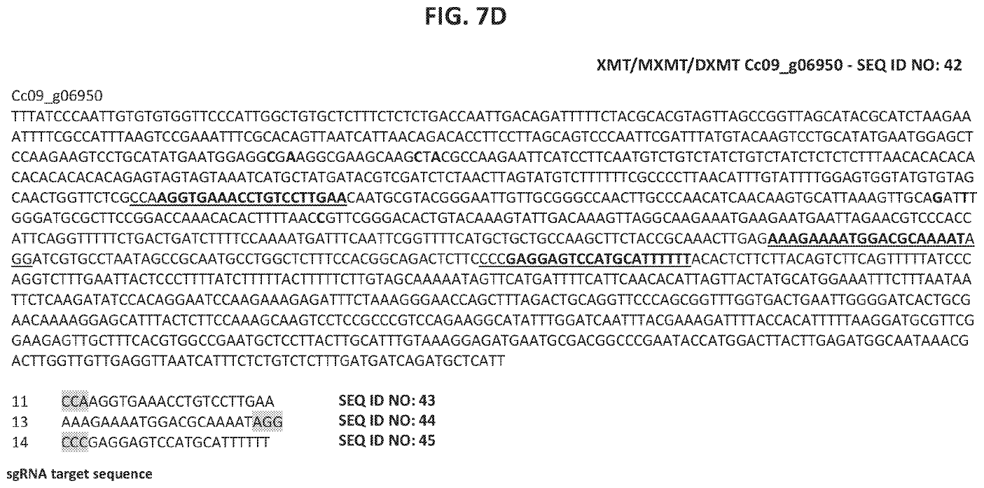

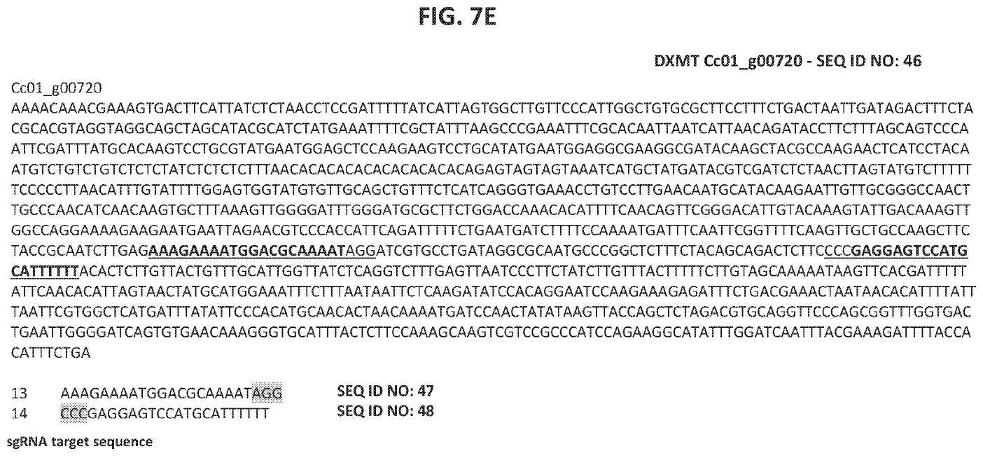

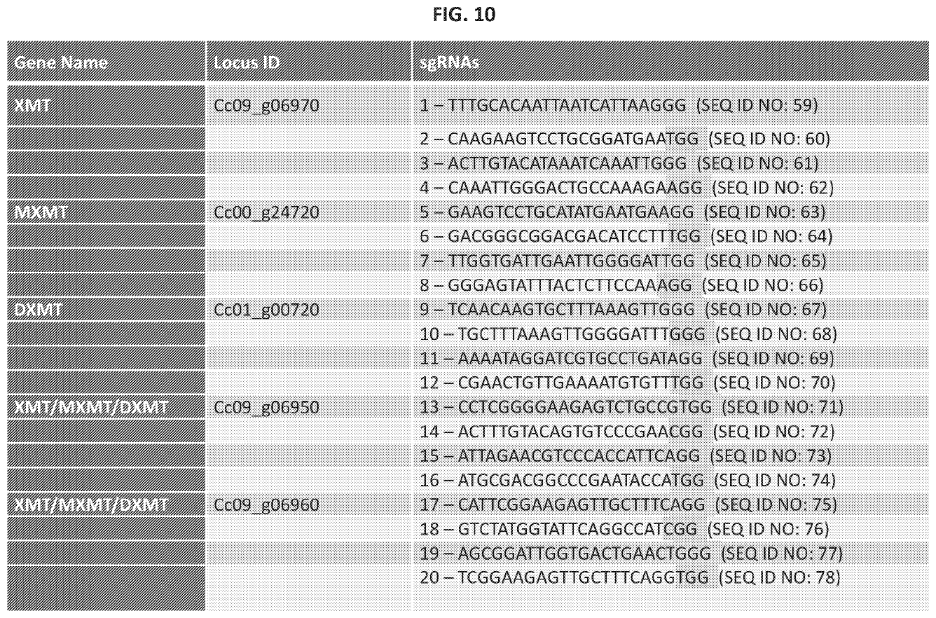

Expression data for each of the individual candidate genes in different coffee tissues was searched utilizing the coffee genome hub (www(dot)coffee-genome(dot)org) (FIGS. 4 and 5). Homologs of XMT, DXMT and MXMT Cc09g06970, Cc01g00720, Cc09g06950, and Cc00g24720 showed moderate to high expression in leaf tissues and perisperm, whereas the gene Cc09g06960 had low expression except for perisperm (FIGS. 4 and 5). Based on these results, one strategy was to design sgRNA that would target Cc09g06970, Cc01g00720, Cc09g06950, and Cc00g24720. However, given the high similarity at the nucleotide level (FIG. 6), conserved areas to which sgRNAs could be designed to target all methyltransferases including Cc09_g06960 were selected. Next, these regions were sequenced to confirm the sequence in the C. canephora lines (marked in bold and underlined in FIGS. 7A-E; SEQ ID NOs: 25-48). Finally, several algorithms were used to design sgRNAs (FIGS. 7A-E, FIG. 10 and SEQ ID NOs: 51-78) and these were ranked according to predicted efficiency and probability to generate a knockout.

XMT, MXMT and DXMT genes (Cc09g06970, Cc09g06950, and Cc09g06960) were targeted with two pairs of sgRNAs as indicated in FIG. 8A. The sgRNAs were positioned between exon 1 and exon 3 of the candidate genes. These regions were selected because they are highly conserved among the aforementioned candidate genes. sgRNAs were cloned into transfection plasmids which contained mCherry, Cas-9, and two sgRNAs driven by a U6 pol 3 promoter.

Next, the CRISPR/Cas9 complex and sgRNAs that target XMT, MXMT and DXMT candidate genes were transfected (as described above using PEG) into coffee protoplasts and enriched for cells that carry such complex by fluorescence-activated cell sorting (FACS). Using the mCherry marker, transfected coffee cells that transiently express the fluorescent protein, Cas9 and the sgRNA were separated, sorted and collected mCherry-positive coffee protoplasts at 3 days post transfection (dpt). DNA was extracted from 5000 sorted protoplasts (Qiagen Plant Dneasy extraction kit) at 6 dpt. Nested PCR was performed for increased sensitivity using primers shown in FIG. 8A. Agarose gels of the amplified region for the candidates XMT, MXMT and DXMT genes are shown in FIG. 8B.

Absence of obvious deletions does not indicate that genome-editing did not take place in the targeted genes. Therefore, to assess whether the sgRNAs and the CRISPR/Cas9 complex was active and induced genome-editing events in XMT, MXMT and DXMT genes, a T7E1 assay was performed. It was found that all sgRNA combinations induced genome-editing events in Cc09g06970, Cc09g06960 genes (FIG. 8C). Moreover, cloning and sequencing confirmed the T7E1 results. Thus, it was found that some of the sgRNAs used induced indels as shown in FIGS. 8D and 8G. The T7E1 assay is more sensitive and therefore, useful to evaluate if the sgRNAs have any activity at all at the targeted genes. In conclusion, these results demonstrate that the CRISPR/Cas9 system can successfully be used to introduce precise mutations in the endogenous XMT, MXMT and DXMT genes and that the design and selection of sgRNAs impact the efficiency of genome-editing.

### Example 3

Regeneration of Transfected Coffee Plants

In parallel to Example 2 above, protoplasts were advanced in the protoplast-regeneration pipeline. Briefly, protoplasts were plated at high density on cellulose membranes on feeder plates to allow for colony formation. Colonies were picked, grown and split into two aliquots. One aliquot was used for DNA extraction and genome editing (GE) testing while the others were kept in culture until their status was verified. Only the ones clearly showing to be GE were selected forward.

Next, proliferating calli were transferred to regeneration media (half strength MS+B5 vitamins, 20 g/l sucrose) for embryo development and germination. 3-4 weeks later, germinating embryos were ready to be transferred to solid medium for seedlings elongation. (FIG. 9A-F).

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

All publications, patents and patent applications mentioned in this specification are herein incorporated in their entirety by into the specification, to the same extent as if each individual publication, patent or patent application was specifically and individually indicated to be incorporated herein by reference. In addition, citation or identification of any reference in this application shall not be construed as an admission that such reference is available as prior art to the present invention. To the extent that section headings are used, they should not be construed as necessarily limiting.

In addition, any priority document(s) of this application is/are hereby incorporated herein by reference in its/their entirety.

What is claimed is:

1. A coffee plant comprising a genome comprising a loss of function mutation in a nucleic acid sequence encoding at least one component of a caffeine biosynthesis pathway.

2. A method of producing a coffee plant or part thereof, the method comprising:
(a) subjecting a coffee plant cell to a DNA editing agent directed at a nucleic acid sequence encoding at least one component of a caffeine biosynthesis pathway to result in a loss of function mutation in said nucleic acid sequence encoding said at least one component of said caffeine biosynthesis pathway; and
(b) regenerating a coffee plant or part thereof from said coffee plant cell.

3. The method of claim 2, further comprising harvesting beans from said coffee plant.

4. The method of claim 2 or 3, further comprising selfing or crossing the coffee plant.

5. The coffee plant of claim 1, or method of any one of claims 2-4, wherein said mutation occurs in at least one allele.

6. The coffee plant of claim 1, or method of any one of claims 2-4, wherein said mutation occurs in all alleles.

7. The coffee plant of claim 1, 5 or 6 or progeny thereof, having been treated with a DNA editing agent directed to said nucleic acid sequence encoding said at least one component of said caffeine biosynthesis pathway.

8. The coffee plant of any one of claim 1 or 5-7, or method of any one of claims 2-6, wherein said mutation is selected from the group consisting of a deletion, an insertion, an insertion/deletion (Indel), and a substitution.

9. The coffee plant of any one of claim 1 or 5-8, or method of any one of claim 2-6 or 8, wherein said coffee plant is from a species Coffea canephora.

10. The coffee plant of any one of claim 1 or 5-8, or method of any one of claim 2-6 or 8, wherein said coffee plant is from a species Coffea arabica.

11. The method of any one of claim 2-6 or 8-10, wherein said subjecting is to a nucleic acid construct encoding said DNA editing agent.

12. The method of any one of claim 2-6 or 8-10, wherein said subjecting is by a DNA-free delivery method.

13. The coffee plant of any one of claim 1 or 5-10, or method of any one of claim 2-6 or 8-12, wherein said coffee plant comprises at least 5% reduction in caffeine as compared to that of a coffee plant of the same genetic background and developmental stage and growth conditions devoid of said loss of function mutation.

14. A nucleic acid construct comprising a nucleic acid sequence encoding a DNA editing agent directed towards at least one component of a caffeine biosynthesis pathway being operably linked to a plant promoter for expressing said DNA editing agent in a cell of a coffee plant.

15. The coffee plant of any one of claim 7-10 or 13, method of any one of claim 2-6 or 8-13, or nucleic acid construct of claim 14, wherein said DNA editing agent comprises at least one sgRNA.

16. The coffee plant, method, or nucleic acid construct of claim 15, wherein said sgRNA comprises a nucleic acid sequence selected from the group consisting of SEQ ID NOs: 51-78.

17. The coffee plant of any one of claim 7-10, 13 or 15-16, method of any one of claim 2-6, 8-13 or 15-16, or nucleic acid construct of any one of claims 14-16, wherein said DNA editing agent does not comprise an endonuclease.

18. The coffee plant of any one of claim 7-10, 13 or 15-16, method of any one of claim 2-6, 8-13 or 15-16, or nucleic acid construct of any one of claims 14-16, wherein said DNA editing agent comprises an endonuclease.

19. The coffee plant of any one of claim 7-10, 13 or 15-18, method of any one of claim 2-6, 8-13 or 15-18, or nucleic acid construct of any one of claims 14-18, wherein said DNA editing agent is of a DNA editing system selected from the group consisting of meganucleases, Zinc finger nucleases (ZFNs), transcription-activator like effector nucleases (TALENs), CRISPR-endonuclease, dCRISPR-endonuclease, and a homing endonuclease.

20. The coffee plant of any one of claim 7-10, 13 or 15-18, method of any one of claim 2-6, 8-13 or 15-18, or nucleic acid construct of any one of claims 14-18, wherein said DNA editing agent is of a DNA editing system comprising CRISPR-Cas.

21. The coffee plant of any one of claim 7-10, 13 or 15-20, method of any one of claim 2-6, 8-13 or 15-20, or nucleic acid construct of any one of claims 14-20, wherein said DNA editing agent is linked to a reporter for monitoring expression in a cell.

22. The coffee plant, method, or nucleic acid construct of claim 21, wherein said reporter is a fluorescent protein.

23. The coffee plant of any one of claim 7-10, 13 or 15-22, method of any one of claim 2-6, 8-13 or 15-22, or nucleic acid construct of any one of claims 14-22, wherein said DNA editing agent is directed to a nucleic sequence that is at least 90% identical between Cc09_g06970 (set forth in SEQ ID NO: 9), Cc09_g06960 (set forth in SEQ ID NO: 7), Cc00_g24720 (set forth in SEQ ID NO: 1), Cc09_g06950 (set forth in SEQ ID NO: 5), Cc01_g00720 (set forth in SEQ ID NO: 3) and Cc02_g09350 (set forth in SEQ ID NO: 11).

24. The coffee plant of any one of claim 7-10, 13 or 15-23, method of any one of claim 2-6, 8-13 or 15-23, or nucleic acid construct of any one of claims 14-23, wherein said DNA editing agent is directed to a nucleic acid segment comprised in a nucleic acid sequence as set forth in any one of SEQ ID NOs: 26-31, 33-36, 38-41, 43-45, 47-48 or 50.

25. The coffee plant of any one of claim 1, 5-10, 13 or 15-24, method of any one of claim 2-6, 8-13 or 15-24, or nucleic acid construct of any one of claims 14-24, wherein said at least one component of a caffeine biosynthesis pathway is a methyltransferase.

26. The coffee plant, method, or nucleic acid construct of claim 25, wherein said methyltransferase comprises a core SAM-binding domain.

27. The coffee plant, method, or nucleic acid construct of claim 25 or 26, wherein said methyltransferase is a N-methyltransferase.

28. The coffee plant, method, or nucleic acid construct of claim 27, wherein said N-methyltransferase is selected from the group consisting of a xanthosine methyltransferase (XMT), a 7-methyxanthine methyltrasferase (MXMT), and 3,7-dimethylxanthine methyltransferase (DXMT).

29. The coffee plant, method, or nucleic acid construct of claim 27, wherein said N-methyltransferase is selected from the group consisting of Cc09_g06970 (set forth in SEQ ID NO: 10), Cc09_g06960 (set forth in SEQ ID NO: 8), Cc00_g24720 (set forth in SEQ ID NO: 2), Cc09_g06950 (set forth in SEQ ID NO: 6), Cc01_g00720 (set forth in SEQ ID NO: 4), Cc02_g09350 (set forth in SEQ ID NO: 12), BAC75663.1 (set forth in SEQ ID NO: 14), ABD90686.1 (set forth in SEQ ID NO: 16), BAB39215.1 (set forth in SEQ ID NO: 18), ABD90685.1 (set forth in SEQ ID NO: 20), BAB39216.1 (set forth in SEQ ID NO: 22), and BAC75664.1 (set forth in SEQ ID NO: 24).

30. The coffee plant of any one of claim 1, 5-10, 13 or 15-29, method of any one of claim 2-6, 8-13 or 15-29, wherein the coffee plant is non-transgenic.

31. A plant part of the coffee plant of any one of claim 1, 5-10, 13 or 15-30.

32. The plant part of claim 31, being a bean.

33. The plant part of claim 32, wherein said bean is dry.

34. A method of producing coffee beans with reduced caffeine content, the method comprising:
(a) growing the plant of any one of claim 1, 5-10, 13 or 15-30; and
(b) harvesting beans from the plant.

35. A method of producing coffee with reduced caffeine content, the method comprising subjecting beans of claim 34 to extraction, dehydration and optionally roasting.

36. Coffee of the beans of any one of claim 3 or 32-33.

37. Coffee of the beans produced by the method of claim 34 or by the method of claim 35.

38. The coffee of claim 36 or 37, being in a powder form.

39. The coffee of claim 36 or 37, being in a granulated form.


SEQUENCE LISTING (86 sequences)

SEQ ID NO: 1 - DNA, 1137 bp, Artificial sequence
  MXMT - Monomethylxanthine methyltransferase 1 nucleic acid sequence
atggagctccaagaagtcctgcatatgaatgaaggtgaaggcgatacaagctacgccaagaatgcatcctacaatctggctcttgccaaggtgaaacctttccttgaacaatgcatacgagaattgttgcgggccaacttgcccaacatcaacaagtgcattaaagttgcggatttgggatgcgcttctggaccaaacacacttttaacagtgcgggacattgtgcaaagtattgacaaagttggccaggaagagaagaatgaattagaacgtcccaccattcagatttttctgaatgatcttttccaaaatgatttcaattcggttttcaagttgctgccaagcttctaccgcaaactcgagaaagaaaatggacgcaagataggatcgtgcctaataagcgcaatgcctggctctttctacggcagactcttccccgaggagtccatgcattttttgcactcttgttacagtgttcattggttatctcaggttcccagcggtttggtgattgaattggggattggtgcaaacaaagggagtatttactcttccaaaggatgtcgtccgcccgtccagaaggcatatttggatcaatttacgaaagattttaccacatttctaaggattcattcgaaagagttgttttcacgtggccgaatgctccttacttgcatttgtaaagtagatgaattcgacgaaccgaatcccctagacttacttgacatggcaataaacgacttgattgttgagggacttctggaggaagaaaaattggatagtttcaatattccattctttacaccttcagcagaagaagtaaagtgcatagttgaggaggaaggttcttgcgaaattttatatctggagacttttaaggcccattatgatgctgccttctctattgatgatgattacccagtaacatcccatgaacaaattaaagcagagtatgtggcatcattaattagatcagtttacgaacccatcctcgcaagtcattttggagaagctattatgcctgacttattccacaggcttgcgaagcatgcagcaaaggttctccacatgggcaaaggctgctataataatcttatcatttctctcgccaaaaagccagagaagtcagacgtgtaa

SEQ ID NO: 2 - protein, 378 aa, Artificial sequence
  MXMT - Monomethylxanthine methyltransferase 1 amino acid sequence
MELQEVLHMNEGEGDTSYAKNASYNLALAKVKPFLEQCIRELLRANLPNINKCIKVADLGCASGPNTLLTVRDIVQSIDKVGQEEKNELERPTIQIFLNDLFQNDFNSVFKLLPSFYRKLEKENGRKIGSCLISAMPGSFYGRLFPEESMHFLHSCYSVHWLSQVPSGLVIELGIGANKGSIYSSKGCRPPVQKAYLDQFTKDFTTFLRIHSKELFSRGRMLLTCICKVDEFDEPNPLDLLDMAINDLIVEGLLEEEKLDSFNIPFFTPSAEEVKCIVEEEGSCEILYLETFKAHYDAAFSIDDDYPVTSHEQIKAEYVASLIRSVYEPILASHFGEAIMPDLFHRLAKHAAKVLHMGKGCYNNLIISLAKKPEKSDV

SEQ ID NO: 3 - DNA, 1155 bp, Artificial sequence
  DXMT - 3,7-dimethylxanthine N-methyltransferase nucleic acid seuence
atggagctccaagaagtcctgcatatgaatggaggcgaaggcgatacaagctacgccaagaactcatcctacaatctgtttctcatcagggtgaaacctgtccttgaacaatgcatacaagaattgttgcgggccaacttgcccaacatcaacaagtgctttaaagttggggatttgggatgcgcttctggaccaaacacattttcaacagttcgggacattgtacaaagtattgacaaagttggccaggaaaagaagaatgaattagaacgtcccaccattcagatttttctgaatgatcttttccaaaatgatttcaattcggttttcaagttgctgccaagcttctaccgcaatcttgagaaagaaaatggacgcaaaataggatcgtgcctgataggcgcaatgcccggctctttctacagcagactcttccccgaggagtccatgcattttttacactcttgttactgtttgcattggttatctcaggttcccagcggtttggtgactgaattggggatcagtgtgaacaaagggtgcatttactcttccaaagcaagtcgtccgcccatccagaaggcatatttggatcaatttacgaaagattttaccacatttctgaggattcattcggaagagttgatttcacgtggccgaatgctccttactttcatttgtaaagaagatgaattcgaccacccgaattccatggacttgcttgagatgtcaataaacgacttggttgttgagggacatctggaggaagaaaaattggatagtttcaatgttccaatctatgcaccttcaacagaagaagtaaagcgcatagttgaggaggaaggttcttttgaaattttatacctggagactttttatgccccttatgatgctggcttctctattgatgatgattaccaaggaagatcccattcccctgtatcctgcgatgaacatgctagagcagcgcatgtggcatctgtcgttagatcaatttacgaacccatcctcgcaagtcattttggagaagctattttacctgacttatcccacaggattgcgaagaatgcagcaaaggttctccgctcgggcaaaggcttctatgatagtgttatcatttctctcgccaaaaagccggagaaggcagacatgtaa

SEQ ID NO: 4 - protein, 384 aa, Artificial sequence
  DXMT - 3,7-dimethylxanthine N-methyltransferase amino acid sequence
MELQEVLHMNGGEGDTSYAKNSSYNLFLIRVKPVLEQCIQELLRANLPNINKCFKVGDLGCASGPNTFSTVRDIVQSIDKVGQEKKNELERPTIQIFLNDLFQNDFNSVFKLLPSFYRNLEKENGRKIGSCLIGAMPGSFYSRLFPEESMHFLHSCYCLHWLSQVPSGLVTELGISVNKGCIYSSKASRPPIQKAYLDQFTKDFTTFLRIHSEELISRGRMLLTFICKEDEFDHPNSMDLLEMSINDLVVEGHLEEEKLDSFNVPIYAPSTEEVKRIVEEEGSFEILYLETFYAPYDAGFSIDDDYQGRSHSPVSCDEHARAAHVASVVRSIYEPILASHFGEAILPDLSHRIAKNAAKVLRSGKGFYDSVIISLAKKPEKADM

SEQ ID NO: 5 - DNA, 1158 bp, Artificial sequence
  DXMT - Probable caffeine synthase 4 nucleic acid sequence
atggagctccaagaagtcctgcatatgaatggaggcgaaggcgaagcaagctacgccaagaattcatccttcaatcaactggttctcgccaaggtgaaacctgtccttgaacaatgcgtacgggaattgttgcgggccaacttgcccaacatcaacaagtgcattaaagttgcagatttgggatgcgcttccggaccaaacacacttttaaccgttcgggacactgtacaaagtattgacaaagttaggcaagaaatgaagaatgaattagaacgtcccaccattcaggtttttctgactgatcttttccaaaatgatttcaattcggttttcatgctgctgccaagcttctaccgcaaacttgagaaagaaaatggacgcaaaataggatcgtgcctaatagccgcaatgcctggctctttccacggcagactcttccccgaggagtccatgcattttttacactcttcttacagtcttcagtttttatcccaggttcccagcggtttggtgactgaattggggatcactgcgaacaaaaggagcatttactcttccaaagcaagtcctccgcccgtccagaaggcatatttggatcaatttacgaaagattttaccacatttttaaggatgcgttcggaagagttgctttcacgtggccgaatgctccttacttgcatttgtaaaggagatgaatgcgacggcccgaataccatggacttacttgagatggcaataaacgacttggttgttgagggacgtctgggggaagaaaaattggacagtttcaatgttccaatctatacagcttcagtagaagaagtaaagtgcatggttgaggaggaaggttcttttgaaattttatacttgcagacttttaagctccgttatgatgctggcttctctattgatgatgattgccaagtaagatcccattccccagaatacagcgatgaacatgctagagcagcgcatgtggcatcattaattagatcagtttacgaacccatcctagcaagtcattttggagaagctattatacctgacatattccacaggtttgcgacgaatgcagcaaaggttatccgcttgggcaaaggcttctataataatcttatcatttctcttgccaaaaaaccagagaagtcagacatataa

SEQ ID NO: 6 - protein, 385 aa, Artificial sequence
  DXMT - Probable caffeine synthase 4 amino acid sequence
MELQEVLHMNGGEGEASYAKNSSFNQLVLAKVKPVLEQCVRELLRANLPNINKCIKVADLGCASGPNTLLTVRDTVQSIDKVRQEMKNELERPTIQVFLTDLFQNDFNSVFMLLPSFYRKLEKENGRKIGSCLIAAMPGSFHGRLFPEESMHFLHSSYSLQFLSQVPSGLVTELGITANKRSIYSSKASPPPVQKAYLDQFTKDFTTFLRMRSEELLSRGRMLLTCICKGDECDGPNTMDLLEMAINDLVVEGRLGEEKLDSFNVPIYTASVEEVKCMVEEEGSFEILYLQTFKLRYDAGFSIDDDCQVRSHSPEYSDEHARAAHVASLIRSVYEPILASHFGEAIIPDIFHRFATNAAKVIRLGKGFYNNLIISLAKKPEKSDI

SEQ ID NO: 7 - DNA, 1158 bp, Artificial sequence
  MXMT - Theobromine synthase 2 nucleic acid sequence
atggagctccaagaagtcctgcatatgaatggaggcgaaggcgatacaagctacgccaagaattcatcgtacaatcaactggttctcaccaaggtgaaacctgtccttgaacaatgcatacgagaattgttgcgggccaacttgcccaacatcaacaagtgcattaaagttgcggatttgggatgcgcttctggaccaaacacacttttaacagttcgggacattgtgcaaagtattgacaaagttggccaggaagagaagaatgaattagaacatcccaccattcaaatttttctgaatgatcttttccaaaatgatttcaattcagttttcaagttgctgccaagcttctaccgcaaactcgagaaagaaaatggacgcaaaataggatcgtgcctaataagcgcaatgcctggctctttctacggcagactcttccccgaggagtccatgcattttttgcactcttgttacagtgttcattggttatctcaggttcccagcggattggtgactgaactggggatcagtgcgaacaaagggatcatttactcttccaaagcaagtcctccgcccgtccagaaggcatatttggaccaatttacaaaagattttaccacatttctgaggattcattcggaagagttgctttcaggtggccgaatgctccttacttgcatttgtaaaggagatgaatccgatggcctgaataccatagacttacttgagagagcaataaacgacttggttgttgagggacttctggaggaagaaaaattggatagtttcaatcttccactctatacaccttcactagaagtagtaaagtgcatagttgaggaggaaggttcttttgaaattttatacctggagacttttaaggtccgttatgatgctggcttctctattgatgatgattaccaagtaagatcccttttccaagtatactgcgatgaacatgttaaagcagcgtatgtgacattcttctttagagcagttttcgaacccatcctcgcaagtcattttggagaagctattatgcctgacttattccacaggtttgcgaagaatgcagcaaaggctctccgcttgggcaacggcttctataatagtcttatcatttctcttgccaaaaaaccagagaagtcagacatgtaa

SEQ ID NO: 8 - protein, 385 aa, Artificial sequence
  MXMT - Theobromine synthase 2 amino acid sequence
MELQEVLHMNGGEGDTSYAKNSSYNQLVLTKVKPVLEQCIRELLRANLPNINKCIKVADLGCASGPNTLLTVRDIVQSIDKVGQEEKNELEHPTIQIFLNDLFQNDFNSVFKLLPSFYRKLEKENGRKIGSCLISAMPGSFYGRLFPEESMHFLHSCYSVHWLSQVPSGLVTELGISANKGIIYSSKASPPPVQKAYLDQFTKDFTTFLRIHSEELLSGGRMLLTCICKGDESDGLNTIDLLERAINDLVVEGLLEEEKLDSFNLPLYTPSLEVVKCIVEEEGSFEILYLETFKVRYDAGFSIDDDYQVRSLFQVYCDEHVKAAYVTFFFRAVFEPILASHFGEAIMPDLFHRFAKNAAKALRLGNGFYNSLIISLAKKPEKSDM

SEQ ID NO: 9 - DNA, 1119 bp, Artificial sequence
  XMT - 7-methylxanthosine synthase 1 nucleic acid sequence
atggagctccaagaagtcctgcggatgaatggaggcgaaggcgatacaagctacgccaagaattcagcctacaatcaactggttctcgccaaggtgaaacctgtccttgaacaatgcgtacgggaattgttgcgggccaacttgcccaacatcaacaagtgcattaaagttgcggatttgggatgcgcttctggaccaaacacacttttaacagttcgggacattgtccaaagtattgacaaagttggccaggaaaagaagaatgaattagaacgtcccaccattcagatttttctgaatgatcttttcccaaatgatttcaattcggttttcaagttgctgccaagcttctaccgcaaacttgagaaagaaaatggacgcaaaataggatcgtgcctaataggggcaatgcccggctctttctacagcagactcttccccgaggagtccatgcattttttacactcttgttactgtcttcaatggttatctcaggttcctagcggtttggtgactgaatcggggatcagtacgaacaaagggagcatttactcttccaaagcaagtcgtctgcccgtccagaaggcatatttggatcaatttacgaaagattttaccacatttctaaggattcattcggaagagttgttttcacatggccgaatgctccttacttgcatttgtaaaggagttgaattagacgcccggaatgccatagacttacttgagatggcaataaacgacttggttgttgagggacatctggaggaagaaaaattggatagtttcaatcttccagtctatataccttcagcagaagaagtaaagtgcatagttgaggaggaaggttcttttgaaattttatacctggagacttttaaggtcctttacgatgctggcttctctattgacgatgaacatattaaagcagagtatgttgcatcttccgttagagcagtttacgaacccatcctcgcaagtcattttggagaagctattatacctgacatattccacaggtttgcgaagcatgcagcaaaggttctccccttgggcaaaggcttctataataatcttatcatttctctcgccaaaaagccagagaagtcagacgtgtaa

SEQ ID NO: 10 - protein, 372 aa, Artificial sequence
  XMT - 7-methylxanthosine synthase 1 amino acid sequence
MELQEVLRMNGGEGDTSYAKNSAYNQLVLAKVKPVLEQCVRELLRANLPNINKCIKVADLGCASGPNTLLTVRDIVQSIDKVGQEKKNELERPTIQIFLNDLFPNDFNSVFKLLPSFYRKLEKENGRKIGSCLIGAMPGSFYSRLFPEESMHFLHSCYCLQWLSQVPSGLVTESGISTNKGSIYSSKASRLPVQKAYLDQFTKDFTTFLRIHSEELFSHGRMLLTCICKGVELDARNAIDLLEMAINDLVVEGHLEEEKLDSFNLPVYIPSAEEVKCIVEEEGSFEILYLETFKVLYDAGFSIDDEHIKAEYVASSVRAVYEPILASHFGEAIIPDIFHRFAKHAAKVLPLGKGFYNNLIISLAKKPEKSDV

SEQ ID NO: 11 - DNA, 1119 bp, Artificial sequence
  DXMT - Probable caffeine synthase 3 nucleic acid sequence
atggaactccaacgagtcctgcacatgagtggaggcgaaggcgatacaagctacgccaaaaattcatcctaccaagtgaagcctgtacttgaacaatgcatacaagaattgttgcggaccaacttaccctacgacgagaagtgcattagagttgctgatttgggatgctcttcaggaccaaacacactattaacagtttcggacatcatacaaagtattgacaaagttagccaggaaatggacaatgaatttgcactgcccacgattcaggtttttctgaatgatcttttcgaaaatgatttcaatacggttatcaagtcgctgccaagcttctaccgcaaacttgaaaaagaaaatggacgcaaaataggatcgtgcctgatagcagcaatgcctggctctttctacggcagactcttccccgagcagtccgtccattttttacactcttcttacagtctccattggttatctcaggttcccaatggtttggtgactgaatcggggatcagtgcgaataaagggagcatttactcttccaaagcaagtcctccggccatccagaaggcatatttggatcaatttacgaaagattttaccacatttctcaggatgcattcggaagagttggtttcacatggccgaatcctcctcactttcatgtgtaaaggagatgaattcgacggcccaaatatcttagacttacttgaggtggcaataaacgacttggttgtcgagggaagtctggaggaagaaaaactggacagtttcaatgttccaatctatgcgccttcagtagaagaagtcaggcacataattgaggaggaacgttcttttgaaattgtatacctggagacgtttaagctccgtcatgatgctggcttctccattgatgataaccaagcagcccatgtggcatcattcgttagagcagcttgggaacctatcctagcaagccattttggagaagctattatagccgacttattccacaggtttgccaagaatgcagcaacgcctctccgcatgggcaaaggcttctttaataatctcatcatttctctcgccaagaaaccacacaagtcagagacatgtaaatatttgtttttagatatgtag

SEQ ID NO: 12 - protein, 387 aa, Artificial sequence
  DXMT - Probable caffeine synthase 3 amino acid sequence
MELQRVLHMSGGEGDTSYAKNSSYQVKPVLEQCIQELLRTNLPYDEKCIRVADLGCSSGPNTLLTVSDIIQSIDKVSQEMDNEFALPTIQVFLNDLFENDFNTVIKSLPSFYRKLEKENGRKIGSCLIAAMPGSFYGRLFPEQSVHFLHSSYSLHWLSQVPNGLVTESGISANKGSIYSSKASPPAIQKAYLDQFTKDFTTFLRMHSEELVSHGRILLTFMCKGDEFDGPNILDLLEVAINDLVVEGSLEEEKLDSFNVPIYAPSVEEVRHIIEEERSFEIVYLETFKLRHDAGFSIDDNQLGSHSQVRFCDEHVRAAHVASFVRAAWEPILASHFGEAIIADLFHRFAKNAATPLRMGKGFFNNLIISLAKKPHKSETCKYLFLDM

SEQ ID NO: 13 - DNA, 1155 bp, Coffea arabica
atggagctccaagaagtcctgcatatgaatggaggcgaaggcgatacaagctacgccaagaactcattctacaatctgtttctcatcagggtgaaacctatccttgaacaatgcatacaagaattgttgcgggccaacttgcccaacatcaacaagtgcattaaagttgcggatttgggatgcgcttctggaccaaacacacttttaacagttcgggacattgtacaaagtattgacaaagttggccaggaaaagaagaatgaattagaacgtcccaccattcagatttttctgaatgatcttttccaaaatgatttcaattcggttttcaagtcgctgccaagcttctaccgcaaacttgagaaagaaaatggacgcaaaataggatcatgcctgataggcgcaatgcctggctctttctacggcagactcttccccgaggagtccatgcattttttacactcttgttactgtttgcattggttatctcaggttcccagcggtttggtgactgaattggggatcagtgcgaacaaagggtgcatttactcttccaaagcaagtcgtccgcccatccagaaggcatatttggatcaatttacgaaagattttaccacatttcttaggattcattcggaagagttgatttcacgtggccgaatgctccttacttggatttgcaaagaagatgaattcgagaacccgaattccatagacttacttgagatgtcaataaacgacttggttattgagggacatctggaggaagaaaaattggacagtttcaatgttccaatctatgcaccttcaacagaagaagtaaagtgcatagttgaggaggaaggttcttttgaaattttatacctggagacttttaaggtcccttatgatgctggcttctctattgatgatgattaccaaggaagatcccattccccagtatcctgcgatgaacatgctagagcagcgcatgtggcatctgtcgttagatcaattttcgaacccatcgtcgcaagtcattttggagaagctatcatgcctgacttatcccacaggattgcgaagaatgcagcaaaggttcttcgctccggcaaaggcttctatgatagtcttatcatttctctcgccaaaaagccagagaagtcagacgtgtaa

SEQ ID NO: 14 - protein, 384 aa, Coffea arabica
MELQEVLHMNGGEGDTSYAKNSFYNLFLIRVKPILEQCIQELLRANLPNINKCIKVADLGCASGPNTLLTVRDIVQSIDKVGQEKKNELERPTIQIFLNDLFQNDFNSVFKSLPSFYRKLEKENGRKIGSCLIGAMPGSFYGRLFPEESMHFLHSCYCLHWLSQVPSGLVTELGISANKGCIYSSKASRPPIQKAYLDQFTKDFTTFLRIHSEELISRGRMLLTWICKEDEFENPNSIDLLEMSINDLVIEGHLEEEKLDSFNVPIYAPSTEEVKCIVEEEGSFEILYLETFKVPYDAGFSIDDDYQGRSHSPVSCDEHARAAHVASVVRSIFEPIVASHFGEAIMPDLSHRIAKNAAKVLRSGKGFYDSLIISLAKKPEKSDV

SEQ ID NO: 15 - DNA, 1155 bp, Coffea canephora
atggagctccaagaagtcctgcatatgaatggaggcgaaggcgatacaagctacgccaagaactcatcctacaatctgtttctcatcagggtgaaacctgtccttgaacaatgcatacaagaattgttgcgggccaacttgcccaacatcaacaagtgctttaaagttggggatttgggatgcgcttctggaccaaacacattttcaacagttcgggacattgtacaaagtattgacaaagttggccaggaaaagaagaatgaattagaacgtcccaccattcagatttttctgaatgatcttttccaaaatgatttcaattcggttttcaagttgctgccaagcttctaccgcaatcttgagaaagaaaatggacgcaaaataggatcgtgcctgataggcgcaatgcccggctctttctacagcagactcttccccgaggagtccatgcattttttacactcttgttactgtttgcattggttatctcaggttcccagcggtttggtgactgaattggggatcagtgtgaacaaagggtgcatttactcttccaaagcaagtcgtccgcccatccagaaggcatatttggatcaatttacgaaagattttaccacatttcttaggattcattcggaagagttgatttcacgtggccgaatgctccttactttcatttgtaaagaagatgaattcgaccacccgaattccatggacttgcttgagatgtcaataaacgacttggttattgagggacatctggaggaagaaaaattggatagcttcaatgttccaatctatgcaccttcaacagaagaagtaaagcgcatagttgaggaggaaggttcttttgaaattttatacctggagacttttaatgccccttatgatgctggcttctctattgatgatgattaccaaggaagatcccattcccctgtatcctgcgatgaacatgctagagcagcgcatgtggcatctgtcgttagatcaatttacgaacccatcctcgcgagtcattttggagaagctattttacctgacttatcccacaggattgcgaagaatgcagcaaaggttctccgctcgggcaaaggcttctatgatagtgttatcatttctctcgccaaaaagccggagaaggcagacatgtaa

SEQ ID NO: 16 - protein, 384 aa, Coffea canephora
MELQEVLHMNGGEGDTSYAKNSSYNLFLIRVKPVLEQCIQELLRANLPNINKCFKVGDLGCASGPNTFSTVRDIVQSIDKVGQEKKNELERPTIQIFLNDLFQNDFNSVFKLLPSFYRNLEKENGRKIGSCLIGAMPGSFYSRLFPEESMHFLHSCYCLHWLSQVPSGLVTELGISVNKGCIYSSKASRPPIQKAYLDQFTKDFTTFLRIHSEELISRGRMLLTFICKEDEFDHPNSMDLLEMSINDLVIEGHLEEEKLDSFNVPIYAPSTEEVKRIVEEEGSFEILYLETFNAPYDAGFSIDDDYQGRSHSPVSCDEHARAAHVASVVRSIYEPILASHFGEAILPDLSHRIAKNAAKVLRSGKGFYDSVIISLAKKPEKADM

SEQ ID NO: 17 - DNA, 1119 bp, Coffea arabica
atggagctccaagaagtcctgcggatgaatggaggcgaaggcgatacaagctacgccaagaattcagcctacaatcaactggttctcgccaaggtgaaacctgtccttgaacaatgcgtacgggaattgttgcgggccaacttgcccaacatcaacaagtgcattaaagttgcggatttgggatgcgcttctggaccaaacacacttttaacagttcgggacattgtccaaagtattgacaaagttggccaggaaaagaagaatgaattagaacgtcccaccattcagatttttctgaatgatcttttcccaaatgatttcaattcggttttcaagttgctgccaagcttctaccgcaaacttgagaaagaaaatggacgcaaaataggatcgtgcctaataggggcaatgcccggctctttctacagcagactcttccccgaggagtccatgcattttttacactcttgttactgtcttcaatggttatctcaggttcctagcggtttggtgactgaattggggatcagtacgaacaaagggagcatttactcttccaaagcaagtcgtctgcccgtccagaaggcatatttggatcaatttacgaaagattttaccacatttctaaggattcattcggaagagttgttttcacatggccgaatgctccttacttgcatttgtaaaggagttgaattagacgcccggaatgccatagacttacttgagatggcaataaacgacttggttgttgagggacatctggaggaagaaaaattggatagtttcaatcttccagtctatataccttcagcagaagaagtaaagtgcatagttgaggaggaaggttcttttgaaattttatacctggagacttttaaggtcctttacgatgctggcttctctattgacgatgaacatattaaagcagagtatgttgcatcttccgttagagcagtttacgaacccatcctcgcaagtcattttggagaagctattatacctgacatattccacaggtttgcgaagcatgcagcaaaggttctccccttgggcaaaggcttctataataatcttatcatttctctcgccaaaaagccagagaagtcagacgtgtaa

SEQ ID NO: 18 - protein, 372 aa, Coffea arabica
MELQEVLRMNGGEGDTSYAKNSAYNQLVLAKVKPVLEQCVRELLRANLPNINKCIKVADLGCASGPNTLLTVRDIVQSIDKVGQEKKNELERPTIQIFLNDLFPNDFNSVFKLLPSFYRKLEKENGRKIGSCLIGAMPGSFYSRLFPEESMHFLHSCYCLQWLSQVPSGLVTELGISTNKGSIYSSKASRLPVQKAYLDQFTKDFTTFLRIHSEELFSHGRMLLTCICKGVELDARNAIDLLEMAINDLVVEGHLEEEKLDSFNLPVYIPSAEEVKCIVEEEGSFEILYLETFKVLYDAGFSIDDEHIKAEYVASSVRAVYEPILASHFGEAIIPDIFHRFAKHAAKVLPLGKGFYNNLIISLAKKPEKSDV

SEQ ID NO: 19 - DNA, 1119 bp, Coffea canephora
atggagctccaagaagtcctgcggatgaatggaggcgaaggcgatacaagctacgccaagaattcagcctacaatcaactggttctcgccaaggtgaaacctgtccttgaacaatgcgtacgggaattgttgcgggccaacttgcccaacatcaacaagtgcattaaagttgcggatttgggatgcgcttctggaccaaacacacttttaacggttcgggacattgtccaaagtattgacaaagttggccaggaaaagaagaatgaattagaacgtcccaccattcagatttttctgaatgatcttttcccaaatgatttcaattcggttttcaagttgctgccaagcttctaccgcaaacttgagaaagaaaatggacgcaaaataggatcgtgcctaataggggcaatgcccggctctttctacagcagactcttccccgaggagtccatgcattttttacactcttgttactgtcttcaatggttatctcaggttcctagcggtttggtgactgaattggggatcggcacgaacaaagggagcatttactcttccaaagcaagtcgtctgcccgtccagaaggcatatttggatcaatttacgaaagattttaccacatttctaaggattcattcggaagagttgttttcacatggccgaatgctccttacttgcatttgtaaaggagttgaattagacgcccggaatgccatagacttacttgagatggcaataaacgacttggttgttgagggacatctggaggaagaaaaattggatagtttcaatcttccagtctatataccttcagcagaagaagtaaagtgcatagttgaggaggaaggttcttttgaaattttatacctggagacttttaaggtcctttacgatgctggcttctctattgacgatgaacatattaaagcagagtatgttgcatcttccgttagagcagtttacgaacccatcctcgcaagtcattttggagaagctattatacctgacatattccacaggtttgcgaagcatgcagcaaaggttctccccttgggcaaaggcttctataataatcttatcatttctctcgccaaaaagccagagaagtcagacatgtaa

SEQ ID NO: 20 - protein, 372 aa, Coffea canephora
MELQEVLRMNGGEGDTSYAKNSAYNQLVLAKVKPVLEQCVRELLRANLPNINKCIKVADLGCASGPNTLLTVRDIVQSIDKVGQEKKNELERPTIQIFLNDLFPNDFNSVFKLLPSFYRKLEKENGRKIGSCLIGAMPGSFYSRLFPEESMHFLHSCYCLQWLSQVPSGLVTELGIGTNKGSIYSSKASRLPVQKAYLDQFTKDFTTFLRIHSEELFSHGRMLLTCICKGVELDARNAIDLLEMAINDLVVEGHLEEEKLDSFNLPVYIPSAEEVKCIVEEEGSFEILYLETFKVLYDAGFSIDDEHIKAEYVASSVRAVYEPILASHFGEAIIPDIFHRFAKHAAKVLPLGKGFYNNLIISLAKKPEKSDM

SEQ ID NO: 21 - DNA, 1137 bp, Coffea arabica
atggagctccaagaagtcctgcatatgaatgaaggtgaaggcgatacaagctacgccaagaatgcatcctacaatctggctcttgccaaggtgaaacctttccttgaacaatgcatacgagaattgttgcgggccaacttgcccaacatcaacaagtgcattaaagttgcggatttgggatgcgcttctggaccaaacacacttttaacagtgcgggacattgtgcaaagtattgacaaagttggccaggaagagaagaatgaattagaacgtcccaccattcagatttttctgaatgatcttttccaaaatgatttcaattcggttttcaagttgctgccaagcttctaccgcaaactcgagaaagaaaatggacgcaagataggatcgtgcctaataagcgcaatgcctggctctttctacggcagactcttccccgaggagtccatgcattttttgcactcttgttacagtgttcattggttatctcaggttcccagcggtttggtgattgaattggggattggtgcaaacaaagggagtatttactcttccaaaggatgtcgtccgcccgtccagaaggcatatttggatcaatttacgaaagattttaccacatttctaaggattcattcgaaagagttgttttcacgtggccgaatgctccttacctgcatttgtaaagtagatgaattcgacgaaccgaatcccctagacttacttgacatggcaataaacgacttgattgttgagggacttctggaggaagaaaaattggatagtttcaatattccattctttacaccttcagcagaagaagtaaagtgcatagttgaggaggaaggttcttgcgaaattttatatctggagacttttaaggcccattatgatgctgccttctctattgatgatgattacccagtaagatcccatgaacaaattaaagcagagtatgtggcatcattaattagatcagtttacgaacccatcctcgcaagtcattttggagaagctattatgcctgacttattccacaggcttgcgaagcatgcagcaaaggttctccacatgggcaaaggctgctataataatcttatcatttctctcgccaaaaagccagagaagtcagacgtgtaa

SEQ ID NO: 22 - protein, 378 aa, Coffea arabica
MELQEVLHMNEGEGDTSYAKNASYNLALAKVKPFLEQCIRELLRANLPNINKCIKVADLGCASGPNTLLTVRDIVQSIDKVGQEEKNELERPTIQIFLNDLFQNDFNSVFKLLPSFYRKLEKENGRKIGSCLISAMPGSFYGRLFPEESMHFLHSCYSVHWLSQVPSGLVIELGIGANKGSIYSSKGCRPPVQKAYLDQFTKDFTTFLRIHSKELFSRGRMLLTCICKVDEFDEPNPLDLLDMAINDLIVEGLLEEEKLDSFNIPFFTPSAEEVKCIVEEEGSCEILYLETFKAHYDAAFSIDDDYPVRSHEQIKAEYVASLIRSVYEPILASHFGEAIMPDLFHRLAKHAAKVLHMGKGCYNNLIISLAKKPEKSDV

SEQ ID NO: 23 - DNA, 1155 bp, Coffea arabica
atggagctccaagaagtcctgcatatgaatgaaggtgaaggcgatacaagctacgccaagaatgcatcctacaatctggctcttgccaaggtgaaacctttccttgaacaatgcatacgagaattgttgcgggccaacttgcccaacatcaacaagtgcattaaagttgcggatttgggatgcgcttctggaccaaacacacttttaacagtgcgggacattgtgcaaagtattgacaaagttggccaggaagagaagaatgaattagaacgtcccaccattcagatttttctgaatgatcttttccaaaatgatttcaattcggttttcaagttgctgccaagcttctaccgcaaactcgagaaagaaaatggacgcaagataggatcgtgcctaataagcgcaatgcctggctctttctacggcagactcttccccgaggagtccatgcattttttgcactcttgttacagtgttcattggttatctcaggttcccagcggtttggtgattgaattggggattggtgcaaacaaagggagtatttactcttccaaagcaagtcgtccgcccgtccagaaggcatatttggatcaatttacgaaagattttaccacatttctaaggattcattcgaaagagttgttttcacgtggccgaatgctccttacttgcatttgtaaagtagatgaatacgacgaaccgaatcccctagacttacttgacatggcaataaacgacttgattgttgagggacatctggaggaagaaaaattggctagtttcaatcttccattctttacaccttcagcagaagaagtaaagtgcatagttgaggaggaaggttcttttgaaattttatacctggagacttttaaggcccattatgatgctggcttctctattgatgatgattacccagtaagatcccatttccaagtatacggcgatgaacatattaaagcagagtatgtggcatcattaattagatcagtttacgaacccatcctcgcaagtcattttggagaagctattatgcctgacttattccacaggcttgcgaagcatgcagcaaaggttctccacttgggcaaaggctgctataataatcttatcatttctctcgccaaaaagccagagaagtcagacgtgtaa

SEQ ID NO: 24 - protein, 384 aa, Coffea arabica
MELQEVLHMNEGEGDTSYAKNASYNLALAKVKPFLEQCIRELLRANLPNINKCIKVADLGCASGPNTLLTVRDIVQSIDKVGQEEKNELERPTIQIFLNDLFQNDFNSVFKLLPSFYRKLEKENGRKIGSCLISAMPGSFYGRLFPEESMHFLHSCYSVHWLSQVPSGLVIELGIGANKGSIYSSKASRPPVQKAYLDQFTKDFTTFLRIHSKELFSRGRMLLTCICKVDEYDEPNPLDLLDMAINDLIVEGHLEEEKLASFNLPFFTPSAEEVKCIVEEEGSFEILYLETFKAHYDAGFSIDDDYPVRSHFQVYGDEHIKAEYVASLIRSVYEPILASHFGEAIMPDLFHRLAKHAAKVLHLGKGCYNNLIISLAKKPEKSDV

SEQ ID NO: 25 - DNA, 1024 bp, Artificial sequence
  XMT Cc09_g06970 - partial nucleic acid sequence
atggagctccaagaagtcctgcggatgaatggaggcgaaggcgatacaagctacgccaagaattcagcctacaatgtctgtctgtctctctatctctctttaacacacacacacacagagtagtagtaaatcatgctatgatacgtcgatctctaacttagtatgtcttttttccccccttaacatttgtattttggagtggtatgtgtagcaactggttctcgccaaggtgaaacctgtccttgaacaatgcgtacgggaattgttgcgggccaacttgcccaacatcaacaagtgcattaaagttgcggatttgggatgcgcttctggaccaaacacacttttaacagttcgggacattgtccaaagtattgacaaagttggccaggaaaagaagaatgaattagaacgtcccaccattcagatttttctgaatgatcttttcccaaatgatttcaattcggttttcaagttgctgccaagcttctaccgcaaacttgagaaagaaaatggacgcaaaataggatcgtgcctaataggggcaatgcccggctctttctacagcagactcttccccgaggagtccatgcattttttacactcttgttactgtcttcaatggttatctcaggtctttgagttaatcccttttatctttttaatttttcttgtagcaaaaatagttcatgattttcattcaacacattagtaactatgcacggaaatttctttaataattctcaagatatccacaggaatccaagaaagagatttctgaagaaactaataacatattttattcaagtcgtggctcatgatttatattcccacatgcaacactaacaaaatgatccaactatataagttaccagctctggacgtgcaggttcctagcggtttggtgactgaatcggggatcagtacgaacaaagggagcatttactcttccaaagcaagtcgtctgcccgtccagaaggcatatttggatcaatttacgaaagattttaccacatttctaaggatt

SEQ ID NO: 26 - DNA, 23 bp, Artificial sequence
  XMT Cc09_g06970 target sequence
ccaaggtgaaacctgtccttgaa

SEQ ID NO: 27 - DNA, 20 bp, Artificial sequence
  XMT Cc09_g06970 target sequence
caagtgcattaaagttgcgg

SEQ ID NO: 28 - DNA, 23 bp, Artificial sequence
  XMT Cc09_g06970 target sequence
ccaaatgatttcaattcggtttt

SEQ ID NO: 29 - DNA, 23 bp, Artificial sequence
  XMT Cc09_g06970 target sequence
aaagaaaatggacgcaaaatagg

SEQ ID NO: 30 - DNA, 23 bp, Artificial sequence
  XMT Cc09_g06970 target sequence
tgcctaataggggcaatgcccgg

SEQ ID NO: 31 - DNA, 23 bp, Artificial sequence
  XMT Cc09_g06970 target sequence
cccgaggagtccatgcatttttt

SEQ ID NO: 32 - DNA, 1400 bp, Artificial sequence
  MXMT Cc09_g06960 - partial nucleic acid sequence
tcattcgtgtctggttcccattggctgtgcgctttctttctgacccattgacagacttttctacgcacgtaactagctggttagcatacgcatctatgaaattttcgctatttaagcccgaaattttgcacaattaatcattaacagacaccttctttagccgtcgcaattcgattgtcctgtatatgaatggagctccaagaagtcctgcatatgaatggaggcgaaggcgatacaagctacgccaagaattcatcgtacaatgtctgtctgtctatctctctctttaacacacacacacacacagagtagtagtaaattatgctatgatacgttgatctctgacttagtatgtcttttttcgccccttaacatttgtattttggagtggtatgtgtagcaactggttctcaccaaggtgaaacctgtccttgaacaatgcatacgagaattgttgcgggccaacttgcccaacatcaacaagtgcattaaagttgcggatttgggatgcgcttctggaccaaacacacttttaacagttcgggacattgtgcaaagtattgacaaagttggccaggaagagaagaatgaattagaacatcccaccattcaaatttttctgaatgatcttttccaaaatgatttcaattcagttttcaagttgctgccaagcttctaccgcaaactcgagaaagaaaatggacgcaaaataggatcgtgcctaataagcgcaatgcctggctctttctacggcagactcttccccgaggagtccatgcattttttgcactcttgttacagtgttcattggttatctcaggtctttgagttaatcccttttatctttttactttttcttgtagcaaaaatagttcatgattttcattcaacacattagtaactatgcatggaaatttctttaataattctaaagacatccacaggaatccaagaaagagatttctgaagaaactaataacatattttatttaagtcgtggctcatgatttatattcccacatgcaacactaacaaaatgatccaactatataagttaccagttctagacgtgcaggttcccagcggattggtgactgaactggggatcagtgcgaacaaagggatcatttactcttccaaagcaagtcctccgcccgtccagaaggcatatttggaccaatttacaaaagattttaccacatttctgaggattcattcggaagagttgctttcaggtggccgaatgctccttacttgcatttgtaaaggagatgaatccgatggcctgaataccatagacttacttgagagagcaataaacgacttggttgttgaggttatcatttctctgtctctttgataatcagatgctcattgcttgttatctgaaataaactaga

SEQ ID NO: 33 - DNA, 23 bp, Artificial sequence
  MXMT Cc09_g06960 target sequence
ccaaggtgaaacctgtccttgaa

SEQ ID NO: 34 - DNA, 23 bp, Artificial sequence
  MXMT Cc09_g06960 target sequence
caacaagtgcattaaagttgcgg

SEQ ID NO: 35 - DNA, 23 bp, Artificial sequence
  MXMT Cc09_g06960 target sequence
aaagaaaatggacgcaaaatagg

SEQ ID NO: 36 - DNA, 23 bp, Artificial sequence
  MXMT Cc09_g06960 target sequence
cccgaggagtccatgcatttttt

SEQ ID NO: 37 - DNA, 1105 bp, Artificial sequence
  MXMT Cc00_g24720 - partial nucleic acid sequence
gtgcgctttctttctgacgaattgacagacttttctacgcacggaggtagctggctagcatacgcatctatgaaattttcgctacttaagcccgaaattttgcacaattaatcattaacagacaccttctttagcagtcgcaattcgattgtcctgcatatgaatggagctccaagaagtcctgcatatgaatgaaggtgaaggcgatacaagctacgccaagaatgcatcctacaatgtctgtctgtctctctatctctctttaacacacacacacacagagaatagtggtaaatcatgctatgatacgtcgatctctaacttcacatttgtattttggactggtatgtgtaacagctggctcttgccaaggtgaaacctttccttgaacaatgcatacgagaattgttgcgggccaacttgcccaacatcaacaagtgcattaaagttgcggatttgggatgcgcttctggaccaaacacacttttaacagtgcgggacattgtgcaaagtattgacaaagttggccaggaagagaagaatgaattagaacgtcccaccattcagatttttctgaatgatcttttccaaaatgatttcaattcggttttcaagttgctgccaagcttctaccgcaaactcgagaaagaaaatggacgcaagataggatcgtgcctaataagcgcaatgcctggctctttctacggcagactcttccccgaggagtccatgcattttttgcactcttgttacagtgttcattggttatctcaggtctttgagttaatcccttttatctttttactttttcttgtagcaaaaatggttcgtgattttcattcaacacattagtaactatgcatggaaatttctttaataattctaaagatatccacaggaatccaagaaagagatttctgaagaaactaataacatattttatctaagtcgtggctcatgatttacattcccacatgcaacactaacaaaatgatccaactatataagttaccagttctggacgtgcaggttcccagcggtttggtgattgaattggggattggtgcaaacaaagggagtatttactcttccaaaggatgtcgtc

SEQ ID NO: 38 - DNA, 23 bp, Artificial sequence
  MXMT Cc00_g24720 target sequence
cctttccttgaacaatgcatacg

SEQ ID NO: 39 - DNA, 23 bp, Artificial sequence
  MXMT Cc00_g24720 target sequence
caacaagtgcattaaagttgcgg

SEQ ID NO: 40 - DNA, 23 bp, Artificial sequence
  MXMT Cc00_g24720 target sequence
aaagaaaatggacgcaagatagg

SEQ ID NO: 41 - DNA, 23 bp, Artificial sequence
  MXMT Cc00_g24720 target sequence
cccgaggagtccatgcatttttt

SEQ ID NO: 42 - DNA, 1315 bp, Artificial sequence
  DXMT Cc09_g06950 - partial nucleic acid sequence
tttatcccaattgtgtgtggttcccattggctgtgctctttctctctgaccaattgacagatttttctacgcacgtagttagccggttagcatacgcatctaagaaattttcgccatttaagtccgaaatttcgcacagttaatcattaacagacaccttccttagcagtcccaattcgatttatgtacaagtcctgcatatgaatggagctccaagaagtcctgcatatgaatggaggcgaaggcgaagcaagctacgccaagaattcatccttcaatgtctgtctatctgtctatctctctctttaacacacacacacacacacacagagtagtagtaaatcatgctatgatacgtcgatctctaacttagtatgtcttttttcgccccttaacatttgtattttggagtggtatgtgtagcaactggttctcgccaaggtgaaacctgtccttgaacaatgcgtacgggaattgttgcgggccaacttgcccaacatcaacaagtgcattaaagttgcagatttgggatgcgcttccggaccaaacacacttttaaccgttcgggacactgtacaaagtattgacaaagttaggcaagaaatgaagaatgaattagaacgtcccaccattcaggtttttctgactgatcttttccaaaatgatttcaattcggttttcatgctgctgccaagcttctaccgcaaacttgagaaagaaaatggacgcaaaataggatcgtgcctaatagccgcaatgcctggctctttccacggcagactcttccccgaggagtccatgcattttttacactcttcttacagtcttcagtttttatcccaggtctttgaattactcccttttatctttttactttttcttgtagcaaaaatagttcatgattttcattcaacacattagttactatgcatggaaatttctttaataattctcaagatatccacaggaatccaagaaagagatttctaaagggaaccagctttagactgcaggttcccagcggtttggtgactgaattggggatcactgcgaacaaaaggagcatttactcttccaaagcaagtcctccgcccgtccagaaggcatatttggatcaatttacgaaagattttaccacatttttaaggatgcgttcggaagagttgctttcacgtggccgaatgctccttacttgcatttgtaaaggagatgaatgcgacggcccgaataccatggacttacttgagatggcaataaacgacttggttgttgaggttaatcatttctctgtctctttgatgatcagatgctcatt

SEQ ID NO: 43 - DNA, 23 bp, Artificial sequence
  DXMT Cc09_g06950 target sequence
ccaaggtgaaacctgtccttgaa

SEQ ID NO: 44 - DNA, 23 bp, Artificial sequence
  DXMT Cc09_g06950 target sequence
aaagaaaatggacgcaaaatagg

SEQ ID NO: 45 - DNA, 23 bp, Artificial sequence
  DXMT Cc09_g06950 target sequence
cccgaggagtccatgcatttttt

SEQ ID NO: 46 - DNA, 1270 bp, Artificial sequence
  DXMT Cc01_g00720 - partial nucleic acid sequence
aaaacaaacgaaagtgacttcattatctctaacctccgatttttatcattagtggcttgttcccattggctgtgcgcttcctttctgactaattgatagactttctacgcacgtaggtaggcagctagcatacgcatctatgaaattttcgctatttaagcccgaaatttcgcacaattaatcattaacagataccttctttagcagtcccaattcgatttatgcacaagtcctgcgtatgaatggagctccaagaagtcctgcatatgaatggaggcgaaggcgatacaagctacgccaagaactcatcctacaatgtctgtctgtctctctatctctctctttaacacacacacacacacacacagagtagtagtaaatcatgctatgatacgtcgatctctaacttagtatgtcttttttcccccttaacatttgtattttggagtggtatgtgttgcagctgtttctcatcagggtgaaacctgtccttgaacaatgcatacaagaattgttgcgggccaacttgcccaacatcaacaagtgctttaaagttggggatttgggatgcgcttctggaccaaacacattttcaacagttcgggacattgtacaaagtattgacaaagttggccaggaaaagaagaatgaattagaacgtcccaccattcagatttttctgaatgatcttttccaaaatgatttcaattcggttttcaagttgctgccaagcttctaccgcaatcttgagaaagaaaatggacgcaaaataggatcgtgcctgataggcgcaatgcccggctctttctacagcagactcttccccgaggagtccatgcattttttacactcttgttactgtttgcattggttatctcaggtctttgagttaatcccttctatcttgtttactttttcttgtagcaaaaataagttcacgatttttattcaacacattagtaactatgcatggaaatttctttaataattctcaagatatccacaggaatccaagaaagagatttctgacgaaactaataacacattttatttaattcgtggctcatgatttatattcccacatgcaacactaacaaaatgatccaactatataagttaccagctctagacgtgcaggttcccagcggtttggtgactgaattggggatcagtgtgaacaaagggtgcatttactcttccaaagcaagtcgtccgcccatccagaaggcatatttggatcaatttacgaaagattttaccacatttctga

SEQ ID NO: 47 - DNA, 23 bp, Artificial sequence
  DXMT Cc01_g00720 target sequence
aaagaaaatggacgcaaaatagg

SEQ ID NO: 48 - DNA, 23 bp, Artificial sequence
  DXMT Cc01_g00720 target sequence
cccgaggagtccatgcatttttt

SEQ ID NO: 49 - DNA, 1319 bp, Artificial sequence
  DXMT Cc02_g09350 - partial nucleic acid sequence
gagtatgtaaatatgtgattgaaaaaaagtactcagattatgtttaacaaatttgccaaaaaaggcaatacatgctgattatattcgaacattccacttttttggaccggaaagagaagggatatgggcatttattgaggatcgtttggaggatgagcagtaatggcatctctgaaagctgtccaatactgacacttggattgattatttgttgaagatgatgattgatgatgatgatgatgataccgatacttggacacttggattgatgatgatgaggaaaggataaatactcctatacaagattgtcgaaaattcgattaagtaagagatcagaaatggaactccaacgagtcctgcacatgagtggaggcgaaggcgatacaagctacgccaaaaattcatcctaccaagtctgtctatccctctttgacaaacacacacgcacgcgcccgcagtcacaggagtggtaacacaagactgtgatacgttgatctctaacatagcgtaccctttttttggctttaacttttttttttttttttttgagtggtgtatgtagaagttggtactgacctaggtgaagcctgtacttgaacaatgcatacaagaattgttgcggaccaacttaccctacgacgagaagtgcattagagttgctgatttgggatgctcttcaggaccaaacacactattaacagtttcggacatcatacaaagtattgacaaagttagccaggaaatggacaatgaatttgcactgcccacgattcaggtttttctgaatgatcttttcgaaaatgatttcaatacggttatcaagtcgctgccaagcttctaccgcaaacttgaaaaagaaaatggacgcaaaataggatcgtgcctgatagcagcaatgcctggctctttctacggcagactcttccccgagcagtccgtccattttttacactcttcttacagtctccattggttatctcaggtttttgaatcaatccctctaatcattttccattttcttgcagcaaagatagttcatgcatgattttcattcaacacattagtaactatgcatggaaggcttctttaacaattctcaagacatccccaagaacccaacccaagaaaagatttctcaagaaatcaacaaccttttttttcttttttttttggtgtggtcgcggctcatgatttgtagtattcccacatgcaattgaccctaacaaaatgctccaacaatgtaacaagttaccagcttgagacgcgtcacgttgacaggttcccaatggtttggtgactgaatcggggatcagtgcgaa

SEQ ID NO: 50 - DNA, 23 bp, Artificial sequence
  DXMT Cc02_g09350 target sequence
aaagaaaatggacgcaaaatagg

SEQ ID NO: 51 - DNA, 20 bp, Artificial sequence
  sgRNA sequence
aaaaccgaattgaaatcatt

SEQ ID NO: 52 - DNA, 20 bp, Artificial sequence
  sgRNA sequence
tgcctaataggggcaatgcc

SEQ ID NO: 53 - DNA, 20 bp, Artificial sequence
  sgRNA sequence
ttcaaggacaggtttcacct

SEQ ID NO: 54 - DNA, 20 bp, Artificial sequence
  sgRNA sequence
caacaagtgcattaaagttg

SEQ ID NO: 55 - DNA, 20 bp, Artificial sequence
  sgRNA sequence
aaagaaaatggacgcaaaat

SEQ ID NO: 56 - DNA, 20 bp, Artificial sequence
  sgRNA sequence
aaaaaatgcatggactcctc

SEQ ID NO: 57 - DNA, 20 bp, Artificial sequence
  sgRNA sequence
cgtatgcattgttcaaggaa

SEQ ID NO: 58 - DNA, 20 bp, Artificial sequence
  sgRNA sequence
aaagaaaatggacgcaagat

SEQ ID NO: 59 - DNA, 23 bp, Artificial sequence
  sgRNA sequence
tttgcacaattaatcattaaggg

SEQ ID NO: 60 - DNA, 23 bp, Artificial sequence
  sgRNA sequence
caagaagtcctgcggatgaatgg

SEQ ID NO: 61 - DNA, 23 bp, Artificial sequence
  sgRNA sequence
acttgtacataaatcaaattggg

SEQ ID NO: 62 - DNA, 23 bp, Artificial sequence
  sgRNA sequence
caaattgggactgccaaagaagg

SEQ ID NO: 63 - DNA, 23 bp, Artificial sequence
  sgRNA sequence
gaagtcctgcatatgaatgaagg

SEQ ID NO: 64 - DNA, 23 bp, Artificial sequence
  sgRNA sequence
gacgggcggacgacatcctttgg

SEQ ID NO: 65 - DNA, 23 bp, Artificial sequence
  sgRNA sequence
ttggtgattgaattggggattgg

SEQ ID NO: 66 - DNA, 23 bp, Artificial sequence
  sgRNA sequence
gggagtatttactcttccaaagg

SEQ ID NO: 67 - DNA, 23 bp, Artificial sequence
  sgRNA sequence
tcaacaagtgctttaaagttggg

SEQ ID NO: 68 - DNA, 23 bp, Artificial sequence
  sgRNA sequence
tgctttaaagttggggatttggg

SEQ ID NO: 69 - DNA, 23 bp, Artificial sequence
  sgRNA sequence
aaaataggatcgtgcctgatagg

SEQ ID NO: 70 - DNA, 23 bp, Artificial sequence
  sgRNA sequence
cgaactgttgaaaatgtgtttgg

SEQ ID NO: 71 - DNA, 23 bp, Artificial sequence
  sgRNA sequence
cctcggggaagagtctgccgtgg

SEQ ID NO: 72 - DNA, 23 bp, Artificial sequence
  sgRNA sequence
actttgtacagtgtcccgaacgg

SEQ ID NO: 73 - DNA, 23 bp, Artificial sequence
  sgRNA sequence
attagaacgtcccaccattcagg

SEQ ID NO: 74 - DNA, 23 bp, Artificial sequence
  sgRNA sequence
atgcgacggcccgaataccatgg

SEQ ID NO: 75 - DNA, 23 bp, Artificial sequence
  sgRNA sequence
cattcggaagagttgctttcagg

SEQ ID NO: 76 - DNA, 23 bp, Artificial sequence
  sgRNA sequence
gtctatggtattcaggccatcgg

SEQ ID NO: 77 - DNA, 23 bp, Artificial sequence
  sgRNA sequence
agcggattggtgactgaactggg

SEQ ID NO: 78 - DNA, 23 bp, Artificial sequence
  sgRNA sequence
tcggaagagttgctttcaggtgg

SEQ ID NO: 79 - DNA, 1399 bp, Artificial sequence
  XMT/MXMT/DXMT mutant seq 2023-3
tcattcgtgtctggttcccattggctgtgcgctttctttctgacccattgacagacttttctacgcacgtaactagctggttagcatacgcatctatgaaattttcgctatttaagcccgaaattttgcacaattaatcattaacagacaccttctttagccgtcgcaattcgattgtcctgtatatgaatggagctccaagaagtcctgcatatgaatggaggcgaaggcgatacaagctacgccaagaattcatcgtacaatgtctgtctgtctatctctctctttaacacacacacacacacagagtagtagtaaattatgctatgatacgttgatctctgacttagtatgtcttttttcgccccttaacatttgtattttggagtggtatgtgtagcaactggttctcaccaagggaaacctgtccttgaacaatgcatacgagaattgttgcgggccaacttgcccaacatcaacaagtgcattaaagttgcggatttgggatgcgcttctggaccaaacacacttttaacagttcgggacattgtgcaaagtattgacaaagttggccaggaagagaagaatgaattagaacatcccaccattcaaatttttctgaatgatcttttccaaaatgatttcaattcagttttcaagttgctgccaagcttctaccgcaaactcgagaaagaaaatggacgcaaaataggatcgtgcctaataagcgcaatgcctggctctttctacggcagactcttccccgaggagtccatgcattttttgcactcttgttacagtgttcattggttatctcaggtctttgagttaatcccttttatctttttactttttcttgtagcaaaaatagttcatgattttcattcaacacattagtaactatgcatggaaatttctttaataattctaaagacatccacaggaatccaagaaagagatttctgaagaaactaataacatattttatttaagtcgtggctcatgatttatattcccacatgcaacactaacaaaatgatccaactatataagttaccagttctagacgtgcaggttcccagcggattggtgactgaactggggatcagtgcgaacaaagggatcatttactcttccaaagcaagtcctccgcccgtccagaaggcatatttggaccaatttacaaaagattttaccacatttctgaggattcattcggaagagttgctttcaggtggccgaatgctccttacttgcatttgtaaaggagatgaatccgatggcctgaataccatagacttacttgagagagcaataaacgacttggttgttgaggttatcatttctctgtctctttgataatcagatgctcattgcttgttatctgaaataaactaga

SEQ ID NO: 80 - DNA, 1399 bp, Artificial sequence; identical to SEQ ID NO: 79
  XMT/MXMT/DXMT mutant seq 2023-6

SEQ ID NO: 81 - DNA, 1022 bp, Artificial sequence
  XMT mutant seq 2023-2
atggagctccaagaagtcctgcggatgaatggaggcgaaggcgatacaagctacgccaagaattcagcctacaatgtctgtctgtctctctatctctctttaacacacacacacagagtagtagtaaatcatgctatgatacgtcgatctctaacttagtatgtcttttttccccccttaacatttgtattttggagtggtatgtgtagcaactggttctcgccaaggtgaaacctgtccttgaacaatgcgtacgggaattgttgcgggccaacttgcccaacatcaacaagtgcattaaagttgcggatttgggatgcgcttctggaccaaacacacttttaacagttcgggacattgtccaaagtattgacaaagttggccaggaaaagaagaatgaattagaacgtcccaccattcagatttttctgaatgatcttttcccaaatgatttcaattcggttttcaagttgctgccaagcttctaccgcaaacttgagaaagaaaatggacgcaaaataggatcgtgcctaataggggcaatgcccggctctttctacagcagactcttccccgaggagtccatgcattttttacactcttgttactgtcttcaatggttatctcaggtctttgagttaatcccttttatctttttaatttttcttgtagcaaaaatagttcatgattttcattcaacacattagtaactatgcacggaaatttctttaataattctcaagatatccacaggaatccaagaaagagatttctgaagaaactaataacatattttattcaagtcgtggctcatgatttatattcccacatgcaacactaacaaaatgatccaactatataagttaccagctctggacgtgcaggttcctagcggtttggtgactgaatcggggatcagtacgaacaaagggagcatttactcttccaaagcaagtcgtctgcccgtccagaaggcatatttggatcaatttacgaaagattttaccacatttctaaggatt

SEQ ID NO: 82 - DNA, 1025 bp, Artificial sequence
  XMT mutant seq 2023-3
atggagctccaagaagtcctgcggatgaatggaggcgaaggcgatacaagctacgccaagaattcagcctacaatgtctgtctgtctctctatctctctttaacacacacacacacagagtagtagtaaatcatgctatgatacgtcgatctctaacttagtatgtcttttttccccccttaacatttgtattttggagtggtatgtgtagcaactggttctcgccaaggatgaaacctgtccttgaacaatgcgtacgggaattgttgcgggccaacttgcccaacatcaacaagtgcattaaagttgcggatttgggatgcgcttctggaccaaacacacttttaacagttcgggacattgtccaaagtattgacaaagttggccaggaaaagaagaatgaattagaacgtcccaccattcagatttttctgaatgatcttttcccaaatgatttcaattcggttttcaagttgctgccaagcttctaccgcaaacttgagaaagaaaatggacgcaaaataggatcgtgcctaataggggcaatgcccggctctttctacagcagactcttccccgaggagtccatgcattttttacactcttgttactgtcttcaatggttatctcaggtctttgagttaatcccttttatctttttaatttttcttgtagcaaaaatagttcatgattttcattcaacacattagtaactatgcacggaaatttctttaataattctcaagatatccacaggaatccaagaaagagatttctgaagaaactaataacatattttattcaagtcgtggctcatgatttatattcccacatgcaacactaacaaaatgatccaactatataagttaccagctctggacgtgcaggttcctagcggtttggtgactgaatcggggatcagtacgaacaaagggagcatttactcttccaaagcaagtcgtctgcccgtccagaaggcatatttggatcaatttacgaaagattttaccacatttctaaggatt

SEQ ID NO: 83 - DNA, 1025 bp, Artificial sequence; identical to SEQ ID NO: 82
  XMT mutant seq 2023-4

SEQ ID NO: 84 - DNA, 1022 bp, Artificial sequence; identical to SEQ ID NO: 81
  XMT mutant seq 2023-6

SEQ ID NO: 85 - DNA, 894 bp, Artificial sequence
  Cc09_g06950_2025 nucleic acid sequence
ccattggctgtgctctttctctctgaccaattgacagatttttctacgcacgtagttagccggttagcatacgcatctaagaaattttcgccatttaagtccgaaatttcgcacagttaatcattaacagacaccttccttagcagtcccaattcgatttatgtacaagtcctgcatatgaatggagctccaagaagtcctgcatatgaatggaggcgaaggcgaagcaagctacgccaagaattcatccttcaatgtctgtctatctgtctatctctctctttaacacacacacacacacacacagagtagtagtaaatcatgctatgatacgtcgatctctaacttagtatgtcttttttcgccccttaacatttgtattttggagtggtatgtgtagcaactggttctcgccaaggaataggatcgtgcctaatagccgcaatgcctggctctttccacggcagactcttccccgaggagtccatgcattttttacactcttcttacagtcttcagtttttatcccaggtctttgaattactcccttttatctttttactttttcttgtagcaaaaatagttcatgattttcattcaacacattagttactatgcatggaaatttctttaataattctcaagatatccacaggaatccaagaaagagatttctaaagggaaccagctttagactgcaggttcccagcggtttggtgactgaattggggatcactgcgaacaaaaggagcatttactcttccaaagcaagtcctccgcccgtccagaaggcatatttggatcaatttacgaaagattttaccacatttttaaggatgcgttcggaagagttgctttcacgtggccgaatgctccttacttgcatttgtaaag

SEQ ID NO: 86 - DNA, 622 bp, Artificial sequence
  Cc09_g06970_2025 nucleic acid sequence
atggagctccaagaagtcctgcggatgaatggaggcgaaggcgatacaagctacgccaagaattcagcctacaatgtctgtctgtctctctatctctctttaacacacacacacacacacacacacacacacagagtagtagtaaatcatgctatgatacgtcgatctctaacttagtatgtcttttttccccccttaacatttgtattttggagtggtatgtgtagcaactggttctcgccaaggtgggataccttctagttaacagtcacaccattcatgacatatccatggaataggatcgtgcctaataggggcaatgcccggctctttctacagcagactcttccccgaggagtccatgcattttttacactcttgttactgtcttcaatggttatctcaggtctttgagttaatcccttttatctttttaatttttcttgtagcaaaaatagttcatgattttcattcaacacattagtaactatgcacggaaatttctttaataattctcaagatatccacaggaatccaagaaagagatttctgaagaaactaataacatattttattcaagtcgtggctcatgatttatattcccacatgcaacactaacaaa